(19) United States
(12) Patent Application Publication
(10) Pub. No.: US 2024/0010991 A1
(43) Pub. Date: Jan. 11, 2024

(54) MATERIALS AND METHODS FOR BIOENGINEERED IPSC POPULATIONS

(71) Applicant: JANSSEN BIOTECH, INC., Horsham, PA (US)

(72) Inventors: Jennifer MCCAFFREY, Lafayette Hill, PA (US); Iqbal S. GREWAL, Newtown, PA (US); Dharmeshkumar PATEL, Lansdale, PA (US); Michael ALLEGREZZA, Ambler, PA (US); Glenn S. COWLEY, Ambler, PA (US); HONGXING SUN, Ambler, PA (US)

(21) Appl. No.: 18/344,973

(22) Filed: Jun. 30, 2023

Related U.S. Application Data
(60) Provisional application No. 63/357,922, filed on Jul. 1, 2022.

(51) Int. Cl. C12N 5/074; C12N 15/11; C12N 9/22; C12N 15/90

(52) U.S. Cl. CPC C12N 5/0696; C12N 15/11; C12N 9/22; C12N 15/907; C12N 2506/11; C12N 2501/2315; C12N 2501/2302

(57) ABSTRACT

Provided herein are methods of producing bioengineered pluripotent stem cells (iPSCs) and isolated populations thereof.

### CROSS-REFERENCE TO RELATED APPLICATIONS

This application claims the benefit of U.S. Provisional Patent Application No. 63/357,922, filed Jul. 1, 2022, which is incorporated by reference herein in its entirety.

### SEQUENCE LISTING

The instant application contains a Sequence Listing which has been submitted electronically in XML file format and is hereby incorporated by reference in its entirety. Said XML copy, created on Jul. 28, 2023, is named JBI6723USNP1_SL.xml and is 36,858 bytes in size.

### 1. FIELD

Provided herein are, inter alia, materials and methods of producing bioengineered induced pluripotent stem cells (iPSCs), and uses thereof.

### 2. BACKGROUND

Pluripotent stem cells such as embryonic stem (ES) cells and induced pluripotent stem cells (iPSCs) possess the proliferative and developmental capacity to differentiate and generate multiple cell types in the body. The scientific potential of these cells is thus uncertain but extraordinary, especially after studies are said to have revealed that gene expression profiles in somatic cells can be changed to epigenetically reprogram them into pluripotent stem cells (see, e.g., Takahashi, K., & Yamanaka, S, Nat. Rev. Mol. Cell Biol., 2016, 17(3):183-93).

Embryonic stem cells can be derived from the inner cell mass of mammalian blastocysts, see, e.g., Human Genes and Genomes: Science, Health, Society (Rosenberg, L. E. & Rosenberg, D. D., 1st ed. 2012). Additionally, somatic cell nuclear transfer (SCNT)-mediated reprogramming has also been utilized to generate pluripotent ES cells, and in some instances, cloned animals (Wilmut, I., et al., Nature, 1997, 385:810-813; Wakayama, T., et al., Nature, 1998, 394:369-374). Nevertheless, SCNT has suffered from various technical (e.g., epigenetic) barriers since the destruction of embryos and introduction of mammalian genetic information into an unfertilized egg is subject to controversies (Matoba, S. & Zhang, Y., supra; Kastenberg, Z. J. & Odorico, J. S., Transplant Rev., 2008, 22(3):215-22).

Alternative techniques to reprogram somatic cells into pluripotent stem cells remain of interest. Induced Pluripotent Stem Cell (iPSC) technologies emerged as one of such alternatives when Yamanaka et al. reported that transcription factors Oct3/4, Sox2, Klf4 and c-Myc may confer pluripotency upon adult somatic cells and for generating iPSCs (Takahashi, K., & Yamanaka, S, Cell, 2006, 126(4):663-76; Wernig, M., et al., Nature, 2007, 448:318-324; Maherali, N., et al., Cell Stem Cell, 2007, 1(1):55-70).

### 3. SUMMARY

In one aspect provided herein is a population of induced pluripotent stem cells (iPSCs), wherein the iPSCs have been, optionally, generated by reprogramming γδ T cells, and the population comprises iPSCs that comprise a disrupted beta-2-microglobulin (B2M) gene. In certain embodiments, the iPSCs are disrupted for both copies of the B2M gene (B2M^{-/-}).

In some aspects, the nucleotide sequence of the B2M gene encodes an amino acid sequence of any one of SEQ ID NOs: 12-16. In some aspects, the nucleotide sequence of the B2M gene encodes an amino acid sequence that is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to any one of SEQ ID NOs: 12-16. In some aspects, the amino acid sequence is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to about or at least about 50 contiguous amino acids in any one of SEQ ID NOs: 12-16. In some aspects, the nucleotide sequence encodes an amino acid sequence of B2M, a portion thereof, or an isoform thereof.

In some aspects, the disrupted B2M gene comprises a deletion of at least a portion of the nucleotide sequence of SEQ ID NO: 1. In some aspects, the deletion is of about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the nucleotide sequence of SEQ ID NO: 1. In some aspects, about or at least about 50%, 55%, 60%, 65%, 70%, 75%, 80%, 85%, 90%, 95%, 96%, 97%, 98%, 99%, or 100% of the iPSCs do not express a detectable level of B2M.

In some aspects, the disruption comprises a deletion of at least one nucleotide base pair. In some aspects, the disruption comprises an insertion of at least one nucleotide base pair. In some aspects, the disrupted B2M gene exhibits reduced B2M expression relative to an undisrupted B2M gene. In some aspects, the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference iPSC.

In some aspects, the iPSCs comprising a disrupted B2M gene exhibit reduced expression of HLA-A, HLA-B, and/or HLA-C as compared to the expression of HLA-A, HLA-B, and/or HLA-C in a reference iPSC, e.g., a reference iPSC that does not comprise a disrupted B2M gene. In some aspects, the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference iPSC. In some aspects, the reference iPSC is a population of iPSCs that do not comprise a disrupted B2M gene. In some embodiments, the reference iPSC is a population of iPSCs in which the B2M gene is not disrupted.

In some aspects, the disrupted B2M gene is generated by contacting the population of iPSCs with an RNA-guided endonuclease or a nucleic acid encoding the RNA-guided endonuclease and a guide RNA (gRNA), and wherein the gRNA binds to a complement sequence of a target motif of a B2M gene. In some aspects, the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c. In some aspects, the RNA-guided endonuclease is Cas12a (Cpf1). In some aspects, the RNA-guided endonuclease is Acidaminococcus sp. BV3L6 Cas12a (Cpf1). In some aspects, the RNA-guided endonuclease is MAD7.

In some aspects, the gRNA binds to at least a portion of a complement sequence of SEQ ID NO:1. In some aspects, the gRNA binds to a complement sequence of any one of SEQ ID NOs: 2-6 or 17. In some aspects, the complement sequence comprises any one of SEQ ID NOs: 19-24. In some aspects, the gRNA comprises a sequence of any one of SEQ ID NOs: 7-11 or 18. In some aspects, the gRNA comprises SEQ ID NO: 18. In some aspects, the gRNA consists of a sequence of any one of SEQ ID NOs: 7-11 or 18. In some aspects, the gRNA consists of SEQ ID NO: 18. In some aspects, the complement sequence comprises any one of SEQ ID NOs: 19-24.

In one aspect an induced pluripotent stem cell (iPSC) comprising a disrupted B2M gene, e.g., a B2M^{-/-} iPSC, is an iPSC produced by a method that comprises: (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with a viral vector encoding one or more reprogramming factors; and (d) culturing the transduced γδ T cells under conditions suitable for reprogramming mammalian somatic cells to a pluripotent state, thereby producing a population of iPSCs. In certain embodiments, an iPSC comprising a disrupted B2M gene is generated by contacting the population of iPSCs thereby produced with an RNA-guided endonuclease or a nucleic acid encoding the RNA-guided endonuclease and a guide RNA (gRNA); wherein the gRNA binds to a target motif of a beta-2-microglobulin (B2M) polynucleotide sequence in the population of iPSCs; wherein the contacting results in cleavage of the B2M polynucleotide sequence, and wherein the B2M gene is disrupted.

In one aspect provided herein is a method of producing induced pluripotent stem cells (iPSCs), wherein the method comprises: (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with a viral vector encoding one or more reprogramming factors; (d) culturing the transduced γδ T cells under conditions suitable for reprogramming mammalian somatic cells to a pluripotent state, thereby producing a population of iPSCs; and (e) contacting the population of iPSCs with an RNA-guided endonuclease or a nucleic acid encoding the RNA-guided endonuclease and a guide RNA (gRNA); wherein the gRNA binds to a target motif of a beta-2-microglobulin (B2M) polynucleotide sequence in the population of iPSCs; wherein the contacting results in cleavage of the B2M polynucleotide sequence, and wherein the B2M gene is disrupted.

In some aspects, the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c. In some aspects, the RNA-guided endonuclease is Acidaminococcus sp. BV3L6 Cas12a (Cpf1). In some aspects, the RNA-guided endonuclease is Cas12a (Cpf1). In some aspects, the RNA-guided endonuclease is MAD7.

In some aspects, the target motif comprises a portion of SEQ ID NO:1. In some aspects, the target motif comprises a sequence of any one of SEQ ID NOs: 2-6 or 17. In some aspects, the target motif consists of a sequence of any one of SEQ ID NOs: 2-6 or 17. In some aspects, the gRNA comprises a sequence of any one of SEQ ID NOs: 7-11 or 18. In some aspects, the gRNA consists of a sequence of any one of SEQ ID NOs: 7-11 or 18. In some aspects, the gRNA comprises SEQ ID NO: 18. In some aspects, the gRNA consists of SEQ ID NO: 18. In some aspects, the gRNA binds to a complement sequence to any one of SEQ ID Nos: 2-6 or 17. In some aspects, the complement sequence comprises any one of SEQ ID NOs: 19-24.

In some aspects, the cleavage of the B2M polynucleotide sequence results in reduced expression of B2M in the iPSCs as compared to the expression of B2M in a reference. In some aspects, the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference. In some aspects, the cleavage of the B2M polynucleotide sequence results in reduced expression of HLA-A, HLA-B, and/or HLA-C as compared to the expression of HLA-A, HLA-B, and/or HLA-C in a reference. In some aspects, the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference. In some aspects, the reference is iPSCs or a population of iPSCs without cleavage of the B2M polynucleotide sequence.

In some aspects, the activation culture further comprises IL-2. In some aspects, the viral vector is a Sendai virus (SeV) vector. In some aspects, the method further comprises obtaining the isolated population of cells from a subject. In some aspects, the cells in the isolated population of cells are peripheral blood mononuclear cells (PBMCs). In some aspects, the cells in the isolated population of cells are terminally differentiated cells. In some aspects, the cells in the isolated population of cells are mammal cells. In some aspects, the cells in the isolated population of cells are human cells. In some aspects, the isolated population of cells are cultured in the activation culture for at most 13 days, at most 10 days, at most 9 days, at most 8 days, at most 7 days, at most 6 days, at most 5 days, at most 4 days, at most 3 days, at most 2 days, or at most 1 day. In some aspects, the isolated population of cells is cultured in the activation culture for at most 3 days. In some aspects, the isolated population of cells is cultured in the activation culture for 3 days. In some aspects, after being cultured in the activation culture the isolated population of cells comprises less than 90%, less than 80%, less than 70%, less than 60%, less than 50%, less than 45%, less than 40%, less than 35%, or less than 30% γδ T cells. In some aspects, after being cultured in the activation culture the isolated population of cells comprises less than 35% γδ T cells. In some aspects, the method further comprises enriching the γδ T cells in the isolated population of cells after step (b). In some aspects, the γδ T cells are enriched by cell-cell clump enrichment. In some aspects, at least part of the γδ T cells are activated to Vγ9⁺ γδ T cells in step (b). In some aspects, at least part of the γδ T cells are activated to Vγ9δ2⁺ γδ T cells in step (b).

In some aspects, the one or more reprogramming factors are selected from a group consisting of OCT3/4, SOX2, KLF4, LIN28, and c-Myc. In some aspects, in step (d) the transduced γδ T cells are cultured in the presence of one or more feeder layers. In some aspects, in step (d) the transduced γδ T cells are cultured in the presence of a mono layer of feeder layer. In some aspects, the feeder layer comprises mouse embryonic fibroblasts (MEFs). In some aspects, the method further comprises isolating and/or purifying the produced iPSCs. In some aspects, the method further comprises differentiating the iPSCs ex vivo to cells of a desired cell type.

In some aspects, the produced iPSCs are negative for a Sendai virus (SeV) vector. In some aspects, the produced iPSCs are derived from γδ T cells. In some aspects, the produced iPSCs have rearrangement genes of TRG and TRD gene loci; and wherein optionally the produced iPSCs have Vγ9 and Vδ2 gene arrangements. In some aspects, the produced iPSCs are not derived from αβ T cells. In some aspects, the produced iPSCs do not produce or express TCRA and/or TCRB or fragments thereof, such that there is no surface expression of TCRA and TCRB, detectable or otherwise.

In some aspects, the produced iPSCs are genomically stable with no loss of a chromosome. In some aspects, the genomic stability of the produced iPSCs is determined by Karyotyping analysis. In some aspects, the produced iPSCs can grow in feeder free medium after adoption. In some aspects, an induced pluripotent stem cell (iPSC) is produced according to the methods. In some aspects, the iPSCs are produced according to the methods. In some aspects, a composition comprises the iPSC. In some aspects, a differentiated IPSC is produced according to the method.

In one aspect provided herein, is a method of producing induced pluripotent stem cells (iPSCs) comprising: (a) a step for performing a function of enriching and/or activating γδ T cells in an isolated population of cells; (b) a step for performing a function of reprogramming the γδ T cells to a pluripotent state, thereby producing iPSCs; and (c) a step for contacting the iPSCs with an RNA-guided endonuclease or a nucleic acid encoding the RNA-guided endonuclease and a guide RNA (gRNA); wherein the gRNA binds to a target motif of a beta-2-microglobulin (B2M) polynucleotide sequence in the iPSCs; and wherein the step for contacting results in cleavage of the B2M polynucleotide sequence. In some aspects, an induced pluripotent stem cell (iPSC) is produced according to the method.

In one aspect provided herein, is an isolated population of induced pluripotent stem cells (iPSCs) comprising pluripotent cells, wherein the pluripotent cells comprise a means for expressing one or more reprogramming factors, and/or wherein the pluripotent cells comprise a means for encoding rearrangement of TRG and TRD genes, and wherein the pluripotent cells comprise a means for cleaving a B2M gene.

### 4. BRIEF DESCRIPTION OF THE FIGURES

FIG. 1 depicts representative phase contrast images of γδ iPSC cells 5 days post electroporation. γδ iPSCs were electroporated with β2m knock out crRNAs along with a nuclease and imaged 5 days post electroporation at 5× and 10× magnifications.

FIG. 2 depicts flow cytometry graphs showing β2m expression on electroporated γδ iPSCs transfected with different gRNAs as compared to untransfected cells. 7 days post electroporation, bulk pool of transfected cells was checked for the reduction in β2m expression using flow cytometry. FIG. 2 shows representative FACS plots showing β2m expression in γδ iPSC B2M KO cells. Respective percentage of each population can be seen on the plots.

FIG. 3 depicts 5×, 10×, and 20×γδ iPSC images 7 days post FACS sorting.

FIG. 4 depicts cell sorting of γδ iPSC double negative population (both β2m and HLA-A, HLA-B, HLA-C) from the total electroporated pool performed 14 days post electroporation.

FIG. 5 depicts flow cytometry showing β2m and HLA-A, HLA-B, HLA-C expression on γδ iPSC cells electroporated with gRNA 1, 2, and 3. 22 days post electroporation, sorted pool of transfected cells was checked for the reduction in β2m and HLA-A, HLA-B, and HLA-C expression using flow cytometry. FIG. 5 shows representative FACS plots showing expression of both β2m and HLA A, HLA-B, and HLA-C in γδ iPSC B2M KO cells as compared to wild-type (WT) γδ iPSCs.

FIG. 6 illustrates targeted insertion of a GFP transgene into B2M in human γδT cell-derived iPSCs (see, inter alia, Example 2).

FIG. 7 illustrates knockout of B2M in human γδT cell-derived iPSCs (see, inter alia, Example 3).

FIG. 8 illustrates targeted insertion of a CAR transgene into B2M in human γδT cell-derived iPSCs (see, inter alia, Example 4).

### 5. DETAILED DESCRIPTION

The present disclosure provides, in part, β2m knockout iPSCs and methods for producing β2m knockout iPSCs, which in turn were derived from T cells, particularly from γδ T cells. In some embodiments, the present disclosure provides, a population of induced pluripotent stem cells (iPSCs), wherein at least some of the iPSCs comprise a disrupted beta-2-microglobulin (B2M) gene, and wherein the iPSCs were generated by reprogramming γδ T cells.

γδ T cells are a subset of T lymphocytes that express TCRs distinctive from those expressed by αβ T cells, a major subset of T lymphocytes in human peripheral blood (Kalyan, S. & Kabelitz, D., Cell Mol. Immunol., 2013, 10(1):21-29). Vγ9Vδ2 T cells are a major subset of γδ T cells, and exhibit significant effector functions against tumor cells (Tyler, C. J., et al., Cellular Immunology, 2015, 296(1):10-21; Silva-Santos. B., Nat Rev Immunol., 2015, 15:683-91).

### 5.1. Definitions

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. The range of quantity, level, value, number, frequency, percentage, dimension, size, amount, weight or length can be ±15%, ±10%, ±9%, ±8%, ±7%, ±6%, ±5%, ±4%, ±3%, ±2%, or ±1% to a reference quantity, level, value, number, frequency, percentage, dimension, size, amount, weight or length. The term “about” in relation to a reference numerical value can include the numerical value itself and a range of values, for example, plus or minus 10% from that numerical value. In some embodiments, the amount “about 10” includes 10 and any amounts from 9 to 11. In some cases, the numerical disclosed throughout can be “about” that numerical value even without specifically mentioning the term “about.”

Unless otherwise indicated, the terms “at least,” “at most,” or “about” preceding a series of elements is to be understood to refer to every element in the series.

As used herein and in the appended claims, the singular forms “a,” “an,” and “the” include plural reference unless the context clearly dictates otherwise.

As used herein, and unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the terms “beta-2 microglobulin,” “B2M,” or “β2m” refer to the beta chain component of MHC class I molecules. Human beta-2 microglobulin is encoded by the B2M gene (e.g., NCBI Gene ID 567). Expression of beta-2 microglobulin is necessary for assembly and function of MHC class I molecules on the cell surface.

As used herein, the term “MHC class I molecule” refers to a major histocompatibility complex (MHC) found on the cell surface which displays peptide fragments of non-self proteins. MHC class I molecules consist of two polypeptide chains. The alpha chain consists of 3 polypeptides referred to as the alpha-1, alpha-2, and alpha-3 domains. The alpha chain is linked non-covalently via the alpha-3 domain to a beta-chain which consists of beta-2 microglobulin (B2M). The alpha chain is polymorphic and is encoded by the HLA gene (i.e., HLA-A, HLA-B, and HLA-C), whereas beta-2 microglobulin is not polymorphic and is encoded by the B2M gene.

As used herein, the term “deletion” or “knock-out,” refers to a genetic modification wherein a site or region of genomic DNA is removed by any molecular biology method, e.g., methods described herein, e.g., by delivering to a site of genomic DNA an endonuclease and at least one gRNA. The term “deletion” or “knock out” includes deleting all or a portion of the target polynucleotide sequence in a way that interferes with the function of the target polynucleotide sequence. In some embodiments, “deletion” or “knock out” can result in complete or partial loss of expression of the target gene. Any number of nucleotides can be deleted. In some embodiments, a deletion involves the removal of at least one, at least two, at least three, at least four, at least five, at least ten, at least fifteen, at least twenty, at least 25, or more than at least 25 nucleotides. In some embodiments, a deletion involves the removal of 10-50, 25-75, 50-100, 50-200, or more than 100 nucleotides. In some embodiments, a deletion involves the removal of an entire target gene, e.g., a B2M gene. In some embodiments, a deletion involves the removal of part of a target gene, e.g., all or part of a promoter and/or coding sequence of a B2M gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell. In some embodiments, a knock out can be achieved by altering a target polynucleotide sequence by inducing an indel in the target polynucleotide sequence in a functional domain of the target polynucleotide sequence (e.g., a DNA binding domain). The term “disruption” or “disrupted” refers to an alteration that results in a gene product that does not exhibit wildtype function and/or level of activity. In some aspects, a disruption refers to an alteration of a gene whereby the disrupted gene results in production of such a non-wildtype gene product. In certain embodiments, the disruption truncates a gene, e.g., a beta-2 microglobulin (B2M) gene. In certain embodiments, the disruption deletes a gene, e.g., a beta-2 microglobulin (B2M) gene. In certain embodiments, the disruption results in the gene producing an inactive protein. In certain embodiments, the disruption results in disruption of the reading frame of B2M by multiple out-of-frame deletions. In certain embodiments, the disruption results in disruption of the reading frame of B2M by a single out-of-frame deletion. In certain embodiments, the disruption results in insertion of about or at least about one, two, three, four, five, six, seven, eight, nine, ten, or more than ten nucleotide(s) or nucleotide base pair(s) (e.g., an insertion that changes the reading frame of a gene (e.g., B2M)). In certain embodiments, the disruption results in disruption of the reading frame of B2M. In particular embodiments, the gene is a B2M gene and the disruption results in the B2M gene producing an inactive B2M protein. In certain embodiments, the disruption results in the gene expressing a reduced amount of gene product, e.g., a reduced amount of B2M polypeptide. In particular embodiments, the gene is a B2M gene and the disruption results in the B2M gene expressing a reduced amount of B2M polypeptide. In certain embodiments, the disruption results in the gene expressing no detectable amount of gene product, e.g., no detectable amount of B2M polypeptide. In certain embodiments, the gene is a B2M gene and the disruption results in the B2M gene expressing no detectable amount of B2M polypeptide. A disrupted gene, e.g., a disrupted B2M gene, may refer to a gene comprising an insertion, deletion, or substitution relative to a corresponding wildtype gene such that the disrupted gene expresses a reduced, e.g., no detectable amount of functional protein relative to expression of the wildtype gene. A gene may be disrupted, for example, via a method of inserting, deleting, or substituting at least one nucleotide/nucleic acid in an endogenous gene such that expression of a functional protein from the endogenous gene is reduced or inhibited. In some embodiments, the terms “disruption,” “disrupted,” “knock-out,” or “deletion” are used interchangeably in the disclosure.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR (clustered regularly interspaced short palindromic repeat)-associated endonuclease. CRISPR clusters contain spacers, sequences complementary to antecedent mobile elements, and target invading nucleic acids. CRISPR clusters are transcribed and processed into CRISPR RNA (crRNA). In some embodiments, an endonuclease is an RNA-guided endonuclease. In certain aspects, the RNA-guided endonuclease is a CRISPR nuclease, e.g., a Type II CRISPR Cas9 endonuclease or a Type V CRISPR Cpf1 (or Cas12a) endonuclease. In some aspects, the RNA-guided endonuclease is Acidaminococcus sp. BV3L6 Cas12a (Cpf1). CRISPR-Cas systems may be characterized as Class 1 or Class 2 systems. Class 1 systems are characterized by multi-subunit effector; that is, comprising multiple Cas proteins. Class 1 systems may be further characterized as Types I, III and IV. Class 2 systems are characterized by a single effector protein having multiple domains. Class 1 systems may be further characterized as Types II, V and VI. For example, Class 2 type II systems include Cas9 while Class 2 type V systems include Cpf1 (Cas12a). Further examples of Cas proteins include, but are not limited to, Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. In some embodiments, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (also known as Cas12a), MAD7, MAD2 endonuclease, or a homolog thereof, a recombination of the naturally occurring molecule thereof, a codon-optimized version thereof, or a modified version thereof, or combinations thereof. Examples of Cas proteins include, but are not limited to, MAD7, MAD2, Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c. In some aspects, the RNA-guided endonuclease is Acidaminococcus sp. BV3L6 Cas12a (Cpf1). In some embodiments, an endonuclease may introduce one or more single-stranded breaks (SSBs) and/or one or more double-stranded breaks (DSBs).

As used herein, the terms “Cas12” or “Cas12 protein” refer to any Cas12 protein including, but not limited to, Cas12 protein such as Cas12a, Cas12b, Cas12c, Cas12d, Cas12e. In some embodiments, a Cas12 protein has an amino acid sequence which is at least 85% (or at least 90%, or at least 95%, or at least 96%, or at least 97%, or at least 98%, or at least 99%) identical to the amino acid sequence of a functional Cas12 protein. In some aspects, the RNA-guided endonuclease is Acidaminococcus sp. BV3L6 Cas12a (Cpf1). In some embodiments, the Cas12 protein may be a Cas12 polypeptide substantially identical to the protein found in nature, or a Cas12 polypeptide having at least about 85% sequence identity (or at least about 90% sequence identity, or at least about 95% sequence identity, or at least about 96% sequence identity, or at least about 97% sequence identity, or at least about 98% sequence identity, or at least about 99% sequence identity) to the Cas12 protein found in nature and having substantially the same biological activity. Examples of Cas12a proteins include, but are not limited to, FnCas12a, AsCas12a, LbCas12a, Lb5Cas12a, HkCas12a, OsCas12a, TsCas12a, BbCas12a, BoCas12a or Lb4Cas12a. Examples of Cas12b proteins include, but are not limited to, AacCas12b, Aac2Cas12b, AkCas12b, AmCas12b, AhCas12b, and AcCas12b.

In some embodiments, the term “Cpf endonuclease” means an RNA-guided DNA endonuclease associated with CRISPR that cleaves a target DNA sequence when coupled with a guide RNA. The Cpf endonuclease is guided by the guide RNA(s) to recognize and cleave a specific target site in double stranded DNA in the genome of a cell. In some embodiments, the CRISPR-Cpf system employs an Acidaminococcus sp. Cpf1 endonuclease, a Lachnospiraceae sp. Cpf1 endonuclease, or a Francisella novicide Cpf1 endonuclease or variant thereof. The Cpf1-crRNA complex cleaves target DNA by identification of a protospacer adjacent motif (PAM) 5′-TTTN for the Acidaminococcus sp. Cpf1 endonuclease and Lachnospiraceae sp. Cpf1 endonuclease, and a PAM sequence 5′-TTN for the Francisella novicide Cpf1. After identification of the PAM, Cpf1 introduces sticky-end DNA double-stranded break of 4-5 nucleotides overhang distal to the 3′ end of the targeted PAM which is then repaired by either non-homologous end joining (NHEJ) or homology-directed repair (HDR). It is understood that the term“Cpf1 endonuclease” encompasses variants thereof.

As known to an ordinarily skilled person in the art the term “Mad endonuclease” means an RNA-guided DNA endonuclease associated with CRISPR that cleaves a target DNA sequence when coupled with a guide RNA. The Mad endonuclease is guided by the guide RNA(s) to recognize and cleave a specific target site in double stranded DNA in the genome of a cell. CRISPR-Mad systems are closely related to the Type V (Cpf1-like) of Class-2 family of CAS enzymes. In some embodiments, the CRISPR-Mad system employs an Eubacterium rectale MAD7 endonuclease or variant thereof. In some embodiments, MAD7 is a Class 2 type V-A CRISPR family identified in Eubacterium rectale. The MAD7-crRNA complex cleaves target DNA by identification of a protospacer adjacent motif (PAM) 5′-YTTN. After identification of the PAM, MAD7 introduces sticky-end DNA double-stranded break of 4-5 nucleotides overhang to the 3′ end of the targeted PAM which is then repaired by either non-homologous end joining (NHEJ) or homology-directed repair (HDR). It is understood that the term “Mad endonuclease” encompasses variants thereof. In some embodiments, the B2M target motif identified or used for CRISPR-Cpf1 (Cas12a) system is the same B2M target motif when using MAD7. In some embodiments, the same guide nucleic acid or guide RNA can be used with a Cpf1 (or Cas12a) and a MAD7 nuclease. In some aspects, the RNA-guided endonuclease is Acidaminococcus sp. BV3L6 Cas12a (Cpf1).

As used herein, the term “guide RNA” or “gRNA” generally refers to short ribonucleic acid that can interact with, e.g., bind to, an endonuclease and bind, or hybridize to a target genomic site or region. In some embodiments, a gRNA is a single-molecule guide RNA (sgRNA). In some embodiments, a gRNA may comprise a spacer extension region. In some embodiments, a gRNA may comprise a tracrRNA extension region. In some embodiments, a gRNA is single-stranded. In some embodiments, a gRNA comprises naturally occurring nucleotides. In some embodiments, a gRNA is a chemically modified gRNA. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, a gRNA may be pre-complexed with a DNA endonuclease. In some embodiments, a gRNA sequence comprises a modification. In some embodiments, the modifications increase the stability of the gRNA. In some embodiments, a gRNA sequence comprises AltR1 and/or AltR2. In some embodiments, AltR1 and AltR2 are proprietary (IDT) modifications used to increase the stability of short RNAs (e.g., gRNA).

As used herein, the term “genetic modification” generally refers to a site of genomic DNA that has been genetically edited or manipulated using any molecular biological method, e.g., methods described herein, e.g., by delivering to a site of genomic DNA an endonuclease and at least one gRNA. Example of genetic modifications include insertions, deletions, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product.

In some embodiments, “alteration,” “modification,” or “at least a partial deletion” of a gene (e.g., B2M gene) results in reduced expression of the target polynucleotide sequence (e.g., compared to cells or population of cells without B2M modification, alteration, or at least partial deletion). The terms “decreased,” “reduced,” and “lower” are all used herein interchangeably to mean a decrease by a statistically significant amount (e.g., two standard deviations (2SD) below normal). In some embodiments, “decreased,” “reduced,” or “lower,” means a decrease by at least about 5% as compared to a reference level, for example a decrease by at least about: 5%, 10%, 15%, 20%, 25%, 30%, 35%, 40%, 45%, 50%, 55%, 60%, 65%, 70%, 75%, 80%, 85%, 90%, 95%, or 100% as compared to a reference level. In some embodiments, “decreased,” “reduced,” or “lower,” is any decrease between 10-100% as compared to a reference level. In some embodiments, “decreased,” “reduced,” or “lower,” means a decrease by at least about: 1-fold, 2-fold, 3-fold, 4-fold, 5-fold, 6-fold, 7-fold, 8-fold, 9-fold, or 10-fold as compared to a reference level. In some embodiments, decreased or reduced expression results in undetectable levels of the target gene or target polynucleotide sequence in a cell or population of cells as determined by a method used by those skilled in the art or a method disclosed in the disclosure (e.g., FACS). In some embodiments, reduced expression of B2M is reduced relative to a reference. In some embodiments, the reference is iPSCs or a population of iPSCs without cleavage of the B2M polynucleotide sequence.

In some embodiments, the terms “increased,” “enhanced,” and “elevated” are all used herein interchangeably to mean an increase by at least about 5% as compared to a reference level, for example an increase by at least about: 5%, 10%, 15%, 20%, 25%, 30%, 35%, 40%, 45%, 50%, 55%, 60%, 65%, 70%, 75%, 80%, 85%, 90%, 95%, or 100% as compared to a reference level. In some embodiments, “increased,” “enhanced,” or “elevated,” is any increase between 10-100% as compared to a reference level. In some embodiments, “increased,” “enhanced,” or “elevated,” means an increase by at least about: 1-fold, 2-fold, 3-fold, 4-fold, 5-fold, 6-fold, 7-fold, 8-fold, 9-fold, or 10-fold as compared to a reference level.

As used herein, the term “polynucleotide,” which may be used interchangeably with the term “nucleic acid” generally refers to a biomolecule that comprises two or more nucleotides. Typically, a polynucleotide of the disclosure is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. In some embodiments, a polynucleotide is a hybrid DNA/RNA molecule. In some embodiments, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated. In some embodiments, a polynucleotide comprises at least two, at least five, at least ten, at least twenty, at least 30, at least 40, at least 50, at least 100, at least 200, at least 250, at least 500, or any number of nucleotides. In some embodiments, a polynucleotide is a site or region of genomic DNA. In some embodiments, a polynucleotide is an endogenous gene that is comprised within the genome of a cell. In some embodiments, a polynucleotide is an exogenous polynucleotide that is not integrated into genomic DNA. In some embodiments, a polynucleotide is an exogenous polynucleotide that is integrated into genomic DNA. In some embodiments, a polynucleotide is a plasmid or an adeno-associated viral vector. In some embodiments, a polynucleotide is a circular or linear molecule.

As used herein, “cell culture medium” (also referred to herein as a “culture medium” or “culture” or “medium”) is a medium for culturing cells containing nutrients that maintain cell viability and support proliferation. The cell culture medium may contain any of the following in any appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Some non-limiting examples are provided herein.

As used herein, “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

As used herein, the term “differentiate,” “differentiation,” or the like refers to the process by which an unspecialized (or uncommitted) or less specialized cell acquires the features of a specialized cell such as, for example, a blood cell or a muscle cell. A differentiated or differentiation-induced cell is one that has taken on a more specialized (or committed) position within the lineage of a cell. A cell is committed when it has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” is intended to mean that the referenced molecule or the referenced activity is introduced into the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously.

As used herein, the term “induced pluripotent stem cells” or, “iPSCs,” refers to stem cells produced from differentiated adult cells that have been induced or changed (i.e., reprogrammed) into cells capable of differentiating into tissues of all three germ or dermal layers: mesoderm, endoderm, and ectoderm.

As used herein, the term “isolated” or the like when used in reference to a cell is intended to mean a cell that is substantially free of at least one component as the referenced cell is found in nature. The term includes a cell that is removed from some or all components as it is found in its natural environment. The term also includes a cell that is removed from at least one, some or all components as the cell is found in non-naturally occurring environments. Therefore, an isolated cell is partly or completely separated from other substances as it is found in nature or as it is grown, stored or subsisted in non-naturally occurring environments. Specific examples of isolated cells include partially pure cells, substantially pure cells, and cells cultured in a medium that is non-naturally occurring.

As used herein, the term “purify” or the like refers to increased purity. For example, the purity can be increased to at least 50%, 60%, 70%, 80%, 90%, 95%, 99%, or 100% (e.g., as compared to a reference).

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “population” when used with reference to T lymphocytes refers to a group of cells including two or more T lymphocytes. The isolated population of T lymphocytes can have only one type of T lymphocyte, or two or more types of T lymphocyte. The isolated population of T lymphocytes can be a homogeneous population of one type of T lymphocyte or a heterogeneous population of two or more types of T lymphocyte. The isolated population of T lymphocytes can also be a heterogeneous population having T lymphocytes and at least a cell other than a T lymphocyte, e.g., a B cell, a macrophage, a neutrophil, an erythrocyte, a hepatocyte, an endothelial cell, an epithelial cell, a muscle cell, a brain cell, etc. The heterogeneous population can have from 0.01% to about 100% T lymphocyte. Accordingly, an isolated population of T lymphocytes can have at least 50%, 60%, 70%, 80%, 90%, 95%, 98%, or 99% T lymphocytes. The isolated population of T lymphocytes can include only one type of T lymphocytes, or a mixture of more than one type of T lymphocytes. The isolated population of T lymphocytes can include one or more, or all of, the different types of T lymphocytes, including but not limited to those disclosed herein. An isolated population of T lymphocytes can include all known types of T lymphocytes. In an isolated population of T lymphocytes that includes more than one type of T lymphocytes, the ratio of each type of T lymphocyte can range from 0.01% to 99.99%. The isolated population also can be a clonal population of T lymphocytes, in which all the T lymphocytes of the population are clones of a single T lymphocyte.

A “recombinant” polynucleotide is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid.

As used herein, “reprogramming,” refers to a process that alters or reverses the differentiation state of a somatic cell. The cell can be either partially or terminally differentiated prior to reprogramming. Reprogramming encompasses complete reversion of the differentiation state of a somatic cell (e.g., a T cell) to a pluripotent state. Reprogramming also encompasses partial reversion of the differentiation state of a somatic cell to a state that renders the cell more susceptible to complete reprogramming to a pluripotent state when subjected to additional manipulations such as those described herein. Such contacting may result in expression of particular genes by the cells, which expression contributes to reprogramming. In certain embodiments of the disclosure, reprogramming of a somatic cell causes the somatic cell to be a pluripotent and ES-like state. The resulting cells are referred to herein as reprogrammed pluripotent somatic cells or induced pluripotent stem cells (iPSCs). In some embodiments, reprogramming also encompasses partial reversion of the differentiation state of a somatic cell to a multipotent state.

Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent. In some embodiments, the methods described herein contribute to establishing the pluripotent state by reprogramming. In some embodiments, the methods described herein may be practiced on cells that fully differentiated and/or particular types of cells (e.g., γδ T cells), rather than on cells that are already multipotent or pluripotent.

As used herein, “reprogramming factor” refers to a gene, RNA, or protein that promotes or contributes to cell reprogramming, e.g., in vitro. Examples of reprogramming factors of interest for reprogramming somatic cells to pluripotency in vitro are Oct3/4, Klf4, c-Myc, Nanog, Sox2, and Lin28, and any gene/protein that can substitute for one or more of these in a method of reprogramming somatic cells, e.g., in vitro.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T lymphocyte can be any T lymphocyte, such as a cultured T lymphocyte, e.g., a primary T lymphocyte, or a T lymphocyte from a cultured T cell line, e.g., Jurkat, SupT1, etc., or a T lymphocyte obtained from a mammal. The T lymphocyte can be CD3+ cells. The T lymphocyte can be any type of T lymphocyte and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. A T lymphocyte can be T regulatory cell, which includes nTregs (natural Tregs), iTregs (inducible Tregs), CD8⁺ Treg, Tr1 regulatory cells, and Th3 cells. Additional types of helper T cells include cells such as Th3 (Treg), Th17, Th9, or Tfh cells. Additional types of memory T cells include cells such as central memory T cells (T_{CM} cells), effector memory T cells (T_{EM} cells and T_{EMRA} cells). The T lymphocyte can also refer to a genetically engineered T lymphocyte, such as a T lymphocyte modified to express a T cell receptor (TCR) or a chimeric antigen receptor (CAR). The T lymphocyte can also be differentiated from a stem cell, definitive hemogenic endothelium, a CD34+ cell, an HSC (hematopoietic stem and progenitor cell), a hematopoietic multipotent progenitor cell, or a T cell progenitor cell.

As used herein, the term “γδ T cells” refers to T cells having T cell receptor comprising a γ-chain and a δ-chain on their surfaces.

As used herein, the term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments, the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an anti-mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. In some embodiments, a feeder free medium comprising a recombinant protein surface is provided. Those of skill in the art would appreciate the use of mediums such as iMatrix/laminin, for example, for use in a feeder-free environment.

“Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder and stromal cells and is also not pre-conditioned by the cultivation of feeder cells. In some embodiments, an additional step is taken to filter the medium to remove non-human elements.

The term “pluripotency associated gene” refers to a gene whose expression under normal conditions (e.g., in the absence of genetic engineering or other manipulation designed to alter gene expression) occurs in and is typically restricted to pluripotent stem cells, and is crucial for their functional identity as such. It will be appreciated that the polypeptide encoded by a gene functionally associated with pluripotency may be present as a maternal factor in the oocyte. The gene may be expressed by at least some cells of the embryo, e.g., throughout at least a portion of the preimplantation period and/or in germ cell precursors of the adult.

The term “pluripotency factor” is used refer to the expression product of pluripotency associated gene, e.g., a polypeptide encoded by the gene. In some embodiments, the pluripotency factor is one that is normally substantially not expressed in somatic cell types that constitute the body of an adult animal (with the exception of germ cells or precursors thereof). For example, the pluripotency factor may be one whose average level in ES cells is at least 50-fold or 100-fold greater than its average level in those terminally differentiated cell types present in the body of an adult mammal. In some embodiments, the pluripotency factor is one that is essential to maintain the viability or pluripotent state of ES cells in vivo and/or ES cells derived using conventional methods. Thus, if the gene encoding the factor is knocked out or inhibited (i.e., its expression is eliminated or substantially reduced), the ES cells are not formed, die or, in some embodiments, differentiate. In some embodiments, inhibiting expression of a gene whose function is associated with pluripotency in an ES cell (resulting in, e.g., a reduction in the average steady state level of RNA transcript and/or protein encoded by the gene by at least 50%, 60%, 70%, 80%, 90%, 95%, or more) results in a cell that is viable but no longer pluripotent. In some embodiments the gene is characterized in that its expression in an ES cell decreases (resulting in, e.g., a reduction in the average steady state level of RNA transcript and/or protein encoded by the gene by at least 50%, 60%, 70%, 80%, 90%, 95%, or more) when the cell differentiates into a terminally differentiated cell.

A “pluripotency inducing gene” as used herein, refers to a gene whose expression, contributes to reprogramming somatic cells to a pluripotent state. “Pluripotency inducing factor” refers to an expression product of a pluripotency inducing gene. A pluripotency inducing factor may, but need not be, a pluripotency factor. Expression of an exogenously introduced pluripotency inducing factor may be transient, i.e., it may be needed during at least a portion of the reprogramming process in order to induce pluripotency and/or establish a stable pluripotent state but afterwards not required to maintain pluripotency. For example, the factor may induce expression of endogenous genes whose function is associated with pluripotency. These genes may then maintain the reprogrammed cells in a pluripotent state.

“Polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

### 5.2. Abbreviations

A list of abbreviations used in the present disclosure is provided in Table 1 below.

TABLE 1
Abbreviations

| Abbreviations | Definitions |
|---|---|
| bFGF | Basic fibroblastic growth factor |
| bp | Base pairs |
| Complete RPMI medium | RPMI + 10% FBS + 1x Pen/Strep |
| CRISPR | Clustered regularly interspaced short |
| | palindromic repeats |
| crRNA | Crispr RNA |
| DAPI | 4′,6-diamidino-2-phenylindole |
| ddH₂O | Double distilled water |
| DMEM | Dulbecco's Modified Eagle Medium |
| DPBS | Dulbecco's phosphate buffered saline |
| ECM | Extracellular matrix |
| FACS | Fluorescence-activated cell sorting |
| FACS buffer | DPBS + 2% FBS |
| 6-FAM | 6-Carboxyfluorescein |
| FBS | Foetal bovine serum |
| FMO | Fluorescence minus one |
| FP/F | Forward Primer |
| gRNA | Guide RNA |
| IU | International Units |
| IDTE | Integrated DNA Technologies |
| KLF4 | Kruppel-like factor 4 |
| KO | Knock-out or knocked-out |
| mAb | Monoclonal antibody |
| MEF | Mouse embryonic fibroblasts |
| Oct-3 | Octamer-binding transcription factor-3 |
| PCR | Polymerase Chain Reaction |
| Pen/Strep | Penicillin and streptomycin |
| PBMCs | Peripheral blood mononuclear cells |
| rhIL-2 | Recombinant human Interleukin-2 |
| rhIL-15 | Recombinant human Interleukin-15 |
| rpm | Revolutions per minute |
| RPMI | Roswell Park Memorial Institute medium |
| RV/R | Reverse primer |
| SeV | Sendai virus |
| SSEA-4 | Stage-specific embryonic antigen-4 |
| MOI | Multiplicity of Infection |
| SOX2 | Sex determining region Y-box 2 |
| TRG | T cell receptor gamma locus |
| TRD | T cell receptor delta locus |
| Tg | Transgenic |
| x g | Gravity (or g-force) |
| V | Voltage |
| Zol | Zoledronic acid monohydrate |

### 5.3. Target Gene

In one aspect, provided herein is a method for altering a target polynucleotide sequence (e.g., B2M) in a cell (e.g., iPSCs derived from γδ T cells (Sections 5.6 and 5.7)) comprising contacting the target polynucleotide sequence with a genome editing method of the disclosure (Section 5.4). In some embodiments, the iPSCs that are used in the disclosure (e.g., to knock out B2M using a gene editing system) and methods of making the same are provided in Sections 5.6 and 5.7 of the disclosure.

In one aspect, provided herein is a method for altering a target polynucleotide sequence in a cell (e.g., IPSCs derived from γδ T cells) using a CRISPR/Cas system (see Section 5.4). Any CRISPR/Cas system that is capable of altering a target polynucleotide sequence in a cell can be used in the disclosure (e.g., to alter B2M in IPSCs derived from γδ T cells). In one aspect, provided herein is a method for altering a target polynucleotide sequence in a cell (e.g., IPSCs derived from γδ T cells) using a TALEN system (see Section 5.4). In one aspect, provided herein is a method for altering a target polynucleotide sequence in a cell (e.g., IPSCs derived from γδ T cells) using a zinc finger system (see Section 5.4). It should be understood that although examples of methods utilizing CRISPR/Cas (e.g., cpf1) are described in detail herein, the disclosure is not limited to the use of these methods/systems. Other methods of targeting, e.g., B2M, to reduce or ablate expression in target cells known to the skilled artisan can be utilized herein.

In one aspect, provided herein is a method for altering a target B2M polynucleotide sequence in a cell comprising contacting at least a portion of a B2M polynucleotide sequence with a clustered regularly interspaced short palindromic repeats-associated (Cas) protein or an endonuclease of the disclosure and at least one ribonucleic acid (e.g., one or two ribonucleic acid(s)), wherein the at least one ribonucleic acid directs the Cas protein or the endonuclease to and hybridize to a target motif of the target B2M polynucleotide sequence. In some embodiments, the target B2M polynucleotide sequence is cleaved.

In some embodiments, altering a target polynucleotide sequence results in a knock out or reduced expression of the target polynucleotide sequence (e.g., B2M) or a portion thereof (e.g., compared to a reference). In some embodiments, a reference is iPSCs or a population of iPSCs without cleavage of the B2M polynucleotide sequence. In some embodiments, altering or knocking out a target polynucleotide sequence is performed in vitro. In some embodiments, altering or knocking out a target polynucleotide sequence is performed ex vivo.

In some embodiments, a target polynucleotide sequence is beta-2-microglobulin (also referred to as B2M or β2m) or a portion thereof. The terms “beta-2-microglobulin,” “B2M,” and “β2m” are used interchangeably in the disclosure. In some embodiments, the target polynucleotide sequence is a variant of B2M. In some embodiments, the target polynucleotide sequence is an isoform of B2M. In some embodiments, the target polynucleotide sequence is a homolog of B2M. In some embodiments, the target polynucleotide sequence is an ortholog of B2M. In some embodiments, the target polynucleotide sequence is beta-2-microglobulin (B2M; Gene ID: 567). In some embodiments, the target polynucleotide sequence is NCBI Reference Sequence: NG_012920.2 or a portion thereof. In some embodiments, the target polynucleotide sequence is SEQ ID NO: 1 or a portion thereof. In some embodiments, the target polynucleotide sequence is B2M from any species (e.g., a mammal, human, mouse, rat, pig, or any other species).

In some embodiments, disruption of B2M or B2M knock out results from deletion of at least a portion of a B2M target polynucleotide sequence (e.g., SEQ ID NO: 1). In some embodiments, a deletion is of about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of a target polynucleotide sequence.

In some embodiments, disruption of B2M or B2M knock out results from insertion of about or at least about one, two, three, four, five, six, seven, eight, nine, ten, or more than ten nucleotide(s) or nucleotide base pair(s) (bp) in the B2M target polynucleotide sequence. In some embodiments, disruption of B2M or B2M knock out results from insertion of about or at least about one, two, three, four, five, six, seven, eight, nine, ten, or more than ten nucleotide(s) or nucleotide base pair(s) in a target polynucleotide sequence, in which such insertion changes the reading frame of B2M. In some embodiments, disruption of B2M or B2M knock out results from insertion of about 1 to about 10 bp, about 1 to about 50 bp, about 1 to about 100 bp, about 1 to about 200 bp, about 1 to about 300 bp, about 1 to about 400 bp, about 1 to about 500 bp, about 1 to about 600 bp, about 1 to about 700 bp, about 1 to about 800 bp, about 1 to about 900 bp, about 1 to about 1000 bp, about 1 to about 5000 bp, about 1 to about 10,000 bp, or more than 10,000 bp.

In some embodiments, the disclosure provides a method of disrupting a gene or a polynucleotide sequence that encodes an amino acid sequence of B2M. In some embodiments, the amino acid sequence of B2M is any one of SEQ ID NOs: 12-16. In some embodiments, the amino acid sequence of B2M is any one of UnitProtKB P61769, UnitProtKB F5H6I0, UnitProtKB H0YLF3, UnitProtKB J3KNU0, and/or UnitProtKB Q16446. In some embodiments, the amino acid sequence is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to any one of SEQ ID NOs: 12-16. In some embodiments, the amino acid sequence is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to about or at least about 50 contiguous amino acids in any one of SEQ ID NOs: 12-16. In some embodiments, the amino acid sequence is about 50 to about 120 amino acids, about 50 to about 200 amino acids, or about 30 to about 300 amino acids. In some embodiments, the amino acid sequence is about, at least about, or at most about 49, 50, 51, 52, 53, 54, 55, 56, 57, 58, 59, 60, 65, 70, 71, 72, 73, 74, 75, 76, 77, 78, 79, 80, 85, 90, 95, 98, 99, 100, 101, 102, 103, 104, 105, 106, 107, 108, 109, 110, 111, 112, 113, 114, 115, 116, 117, 118, 119, 120, or more than 120 amino acids (e.g., of any one of SEQ ID NOs: 12-16). In some embodiments, the nucleotide sequence encodes an amino acid sequence of B2M, a portion thereof, or an isoform thereof.

In some embodiments, altering a target polynucleotide sequence comprises contacting at least a portion of a polynucleotide sequence (e.g., B2M) with an endonuclease of the disclosure and at least one ribonucleic acid (e.g., gRNA), wherein the at least one ribonucleic acid directs the endonuclease to and hybridize to a target motif of the target B2M polynucleotide sequence. In some embodiments, the target motif is a portion of the target polypeptide. In some embodiments, the target motif is a portion of NCBI Reference Sequence: NG_012920.2. In some embodiments, the target motif is a portion of the target polypeptide. In some embodiments, the target motif is a portion of B2M. In some embodiments, the target motif is a portion of SEQ ID NO: 1. In some embodiments, the target motif is about, at least about, or at most about: 5 nucleotides (NTs), 10 NTs, 15 NTs, 16 NTs, 17 NTs, 18 NTs, 19 NTs, 20 NTs, 21 NTs, 22 NTs, 23 NTs, 24 NTs, 25 NTs, 26 NTs, 27 NTs, 28 NTs, 29 NTs, 30, 31 NTs, 32 NTs, 33 NTs, 34 NTs, 35 NTs, 36 NTs, 37 NTs, 38 NTs, 39 NTs, 40 NTs, 45 NTs, 50 NTs, 55 NTs, 60 NTs, or more than 60 nt. In some embodiments, the target motif is between about 15 NTs and about 20 NTs sequence, between about 15 NTs and about 25 NTs sequence, between about 15 NTs and about 30 NTs sequence, between about 15 NTs and about 35 NTs sequence, between about 15 NTs and about 40 NTs sequence, between about 15 NTs and about 45 NTs sequence, between about 15 NTs and about 50 NTs sequence, between about 20 NTs and about 25 NTs sequence, between about 20 NTs and about 30 NTs sequence, between about 20 NTs and about 35 NTs sequence, between about 20 NTs and about 40 NTs sequence, between about 20 NTs and about 45 NTs sequence, or between about 20 NTs and about 50 NTs sequence. In some embodiments, the target motif is between about 20 NTs and about 24 NTs sequence. In some embodiments, the target motif is about, at least about, or at most about 20 NTs. In some embodiments, the target motif is about 21 NTs. In some embodiments, the target motif is about 22 NTs. In some embodiments, the target motif is about 23 NTs. In some embodiments, the target motif is about 24 NTs. In some embodiments, the target motif is about 25 NTs. In some embodiments, the target motif is about 26 NTs. In some embodiments, the target motif is about 27 NTs. In some embodiments, the target motif is about 28 NTs. In some embodiments, the target motif is about 29 NTs. In some embodiments, the target motif is about 30 NTs.

In some embodiments, the target motif comprises at least one mutation, substitution, addition, and/or deletion relative to a portion of the target polypeptide. In some embodiments, the target motif is about, at least about, or at most about 75%, 80%, 85%, 90%, 91%, 92%, 93%, 94%, 95%, 96%, 97%, 98%, 99%, or 100% identical to a portion of the target polypeptide. In some embodiments, the target motif comprises or consists of one or more than one nucleotide modification(s) (e.g., nucleotide substitution, addition, deletion, and/or insertion) relative to a portion of the target polypeptide. In some embodiments, the target motif comprises or consists of about 1, 2, 3, 4, 5, or more than 5 nucleotide modification(s) relative to a portion of the target polypeptide.

In some embodiments, the target motif is selected to minimize off-target effects of the CRISPR/Cas system or any gene or genome editing of the disclosure. Those skilled in the art will appreciate that a variety of techniques can be used to select suitable target motifs for minimizing off-target effects (e.g., bioinformatics analyses). In some embodiments, the target motif is selected such that it contains at least one mismatch (e.g., at least two, three, four, or more mismatches) when compared with a genomic nucleotide sequence in a cell.

In some embodiments, the target motif comprises any one of SEQ ID NOs: 2-6 or 17. In some embodiments, the target motif consists of any one of SEQ ID NOs: 2-6 or 17. In some embodiments, the target motif comprises or consists of at least one nucleotide modification relative to any one of SEQ ID NOs: 2-6 or 17. In some embodiments, the target motif comprises or consists of about 1, 2, 3, 4, 5, or more than 5 nucleotide modification(s) relative to any one of SEQ ID NOs: 2-6 or 17. In some embodiments, the target motif is about, at least about, or at most about 75%, 80%, 85%, 90%, 91%, 92%, 93%, 94%, 95%, 96%, 97%, 98%, 99%, or 100% identical to any one of SEQ ID NOs: 2-6 or 17. In some embodiments, the target motif comprises a sequence comprising at least one, two, three, four, five, or more than five nucleotide mismatches compared to any one of SEQ ID NOs: 2-6 or 17. In some embodiments, the complement sequence comprises any one of SEQ ID NOs: 19-24.

### 5.4. Gene or Genome Editing

In some embodiments, genome editing methods as described herein, e.g., the CRISPR-endonuclease system, may be used to genetically modify a cell as described herein, e.g., to create B2M knock-out cells (B2M knock-out iPSC cells derived from γδ T cells). In some embodiments, genome editing methods as described herein, e.g., the CRISPR-endonuclease system, is used to genetically modify a cell as described herein, e.g., to introduce at least one genetic modification within or near at least one gene (e.g., B2M).

In some embodiments, any known method of genome editing known to a skilled person can be used to generate a B2M knock-out cell of the disclosure. In some embodiments, methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ). NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, which has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome (Cho and Greenberg, Nature, 2015, 518, 174-76; Kent et al., Nature Structural and Molecular Biology, 2015, 22(3):230-7; Mateos-Gomez et al., Nature, 2015, 518, 254-57; Ceccaldi et al., Nature, 2015, 528, 258-62).

Each of these genome editing mechanisms can be used to create desired genetic modifications. A step in the genome editing process can be to create one or two DNA breaks, the latter as double-strand breaks or as two single-stranded breaks, in the target locus at near the site of intended mutation or alteration. This can be achieved via the use of an endonuclease, as described herein.

In some embodiments, a target gene or a target polypeptide of the disclosure (e.g., B2M) is disrupted or at least partially deleted via a CRISPR-Cas system. In some embodiments, the CRISPR/Cas system that is used to alter target polynucleotide sequences in cells include RNA binding proteins, endo- and exo-nucleases, helicases, and/or polymerases. In some embodiments, the CRISPR-endonuclease system comprises an endonuclease and at least one guide nucleic acid that directs DNA cleavage of the endonuclease by hybridizing to a recognition site (or target motif of a target polynucleotide) in the genomic DNA. In some embodiments, the gRNA of the embodiments herein bind to the complementary strand of the target gene. In some aspects, the complement sequence comprises any one of SEQ ID NOs: 19-24. In some embodiments, the CRISPR-endonuclease system comprises an endonuclease and at least one ribonucleic acid (e.g., guide RNA (gRNA)) that directs DNA cleavage of the endonuclease by hybridizing to a recognition site (or target motif of a target polynucleotide) in the genomic DNA. One of skill in the art would appreciate that the gRNA is guided to a target motif and binds its complementary sequence in a target target motif. In some aspects, the complement sequence comprises any one of SEQ ID NOs: 19-24. In some embodiments herein a disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene. The target motif comprises a double strand nucleic acid region in which the gRNA binds to one of the strands, the complementary strand to the gRNA. In some embodiments, the CRISPR system is a Type I, II, III, IV, V, and/or VI system(s). In some embodiments, the CRISPR system is a Type II CRISPR/Cas9 system. In some embodiments, the CRISPR system is a Type V CRISPR/Cpf1 (or Cas12a) system. In some embodiments, the CRISPR system is a CRISPR-MAD7 system. In some embodiments, the CRISPR system includes an endonuclease, e.g., Cas9, Cpf1, or MAD7, and one or two noncoding RNAs-crisprRNA (crRNA) and trans-activating RNA (tracrRNA) to target the cleavage of DNA.

In some embodiments, methods of genome editing of the disclosure uses at least one and/or any ribonucleic acid (e.g., gRNA) that is capable of directing an endonuclease (Cas protein) to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, the CRISPR RNA (crRNA) is or comprises about 17-20 nucleotide sequence complementary to the target DNA (target motif of a target polynucleotide). In some embodiments, tracr RNA serves as a binding scaffold for the endonuclease (e.g., Cas9, Cpf1, MAD7, or any other endonuclease of the disclosure). In some embodiments, a single ribonucleic acid comprises a guide RNA that directs the endonuclease or Cas protein to and hybridizes to a target motif of the target polynucleotide sequence in a cell. In some embodiments, at least one of the ribonucleic acids comprises a guide RNA that directs the endonuclease or Cas protein to and hybridizes to a target motif of the target polynucleotide sequence in a cell. In some embodiments, both of the one to two ribonucleic acids comprise a guide RNA that directs the endonuclease or Cas protein to and hybridizes to a target motif of the target polynucleotide sequence in a cell. In some embodiments, the at least one ribonucleic acid(s) of the present disclosure can be selected to hybridize to a variety of different target motifs, for example, different target motifs within a target polynucleotide. In some embodiments, the at least one ribonucleic acid(s) of the present disclosure can be selected to hybridize to a variety of different target motifs depending on the particular CRISPR/Cas system employed, and the sequence of the target polynucleotide, as will be appreciated by those skilled in the art. In some embodiments, the at least one ribonucleic acid(s) (e.g., one to two ribonucleic acids) can also be selected to minimize hybridization with nucleic acid sequences other than the target polynucleotide sequence. In some embodiments, the at least one ribonucleic acid(s) (e.g., one to two ribonucleic acids) hybridizes to a target motif that contains at least two mismatches when compared with all other genomic nucleotide sequences in the cell. In some embodiments, the at least one ribonucleic acid(s) (e.g., one to two ribonucleic acids) hybridizes to a target motif that contains at least one mismatch when compared with all other genomic nucleotide sequences in the cell. In some embodiments, the at least one ribonucleic acid(s) (e.g., one to two ribonucleic acids) are designed to hybridize to a target motif immediately adjacent to a deoxyribonucleic acid motif recognized by the endonuclease or Cas protein. In some embodiments, the at least one ribonucleic acid(s) (e.g., one to two ribonucleic acids) is designed to hybridize to a target motif immediately adjacent to a deoxyribonucleic acid motif recognized by the endonuclease or Cas protein which flank a mutant allele located between the target motifs.

In some embodiments, the at least one ribonucleic acid (e.g., guide RNA) is complementary to and/or hybridize to a sequence on the same strand of a target polynucleotide sequence (e.g., B2M). In some embodiments, the at least one ribonucleic acid (e.g., guide RNA) is complementary to and/or hybridize to a sequence on the opposite strand of a target polynucleotide sequence. In some embodiments the at least one ribonucleic acid (e.g., guide RNA) is not complementary to and/or do not hybridize to a sequence on the opposite strand of a target polynucleotide sequence. In some embodiments, the at least one ribonucleic acid (e.g., guide RNA) is complementary to and/or hybridize to overlapping target motifs of a target polynucleotide sequence. In some embodiments herein, the gRNA of the instant application may bind to the complementary sequence in a target motif. A target motif comprises a double strand nucleic acid, in which a gRNA will bind to one of the strands to which it is complementary. In some embodiments the at least one ribonucleic acid (e.g., guide RNA) is complementary to and/or hybridize to offset target motifs of a target polynucleotide sequence. In some aspects, the complement sequence comprises any one of SEQ ID NOs: 19-24.

In some embodiments, the CRISPR endonuclease is a Cas9, and/or a Cpf1, e.g., L. bacterium ND2006 Cpf1 and/or Acidaminococcus sp. BV3L6 Cpf1 (Example 1, Section 7), and or a MAD7, and in various embodiments CRISPR/MAD7 is used (inter alia, in Example 1 of the disclosure, for example, MAD7 wherein the same B2M target motif and gRNA sequences (Section 7 of the disclosure) are used with other Type V CRISPR enzymes). In some embodiments, since MAD7 is a Cas12a-like endonuclease, the B2M target motif and/or the guide nucleic acid (e.g., gRNA) used or identified for Cpf1 or Cas-12a is the same as the B2M target motif and/or the guide nucleic acid (e.g., gRNA) used for MAD7. In some embodiments, the B2M target motif identified or used for CRISPR-Cpf1 system is the same B2M target motif used for CRISPR-MAD7 system. In some embodiments, the guide nucleic acid (e.g., gRNA) identified or used for CRISPR-Cpf1 system is the same guide nucleic acid (e.g., gRNA) used for CRISPR-MAD7 system. In some embodiments, the B2M target motif and the guide nucleic acid (e.g., gRNA) identified or used for CRISPR-Cpf1 system is the same B2M target motif and the same guide nucleic acid (e.g., gRNA) used for CRISPR-MAD7 system. In some embodiments, the CRISPR endonuclease is MAD7. In some embodiments, the nuclease used in the methods of the disclosure is Inscripta's MAD7™ Nuclease. In some embodiments, the nuclease used in the methods of the disclosure is an Inscripta's nuclease. In some embodiments, the CRISPR endonuclease is a Cas9 (CRISPR associated protein 9). In some embodiments, the Cas9 endonuclease is from Streptococcus pyogenes. In some embodiments, other Cas9 homologs is used, e.g., S. aureus Cas9, N. meningitidis Cas9, S. thermophilus CRISPR 1 Cas9, S. thermophilus CRISPR 3 Cas9, or T. denticola Cas9. In some embodiments, the endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and/or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of an endonuclease can be used. In some embodiments, the endonuclease is any one or more endonuclease of the disclosure. In some embodiments, the endonuclease is any one or more endonuclease known to a skilled person. In some embodiments, exogenous Cas protein can be introduced into the cell in polypeptide form. In certain embodiments, a Cas protein can be conjugated to or fused to a cell-penetrating polypeptide or cell-penetrating peptide. As used herein, “cell-penetrating polypeptide” and “cell-penetrating peptide” refer to a polypeptide or peptide, respectively, which facilitates the uptake of molecule into a cell. In some embodiments, the cell-penetrating polypeptides can contain a detectable label.

In some embodiments, the endonuclease or a Cas protein can be conjugated to or fused to a charged protein (e.g., that carries a positive, negative, or overall neutral electric charge). Such linkage may be covalent. In some embodiments, the endonuclease or Cas protein can be fused to a superpositively charged GFP to significantly increase the ability of the Cas protein to penetrate a cell (Cronican et al. ACS Chem Biol. 2010; 5(8):747-52). In some embodiments, the endonuclease or Cas protein can be fused to a protein transduction domain (PTD) to facilitate its entry into a cell. Exemplary PTDs include Tat, oligoarginine, and penetratin. In some embodiments, the endonuclease or Cas protein comprises a Cas polypeptide fused to a cell-penetrating peptide.

In some embodiments, the endonuclease is linked to at least one nuclear localization signal (NLS). The at least one NLS can be located at or within 50 amino acids of the amino-terminus of the endonuclease and/or at least one NLS can be located at or within 50 amino acids of the carboxy-terminus of the endonuclease.

In some embodiments, the CRISPR-endonuclease system comprises an RNA-guided endonuclease. In some embodiments, an RNA-guided endonuclease comprises an amino acid sequence having at least about 10%, at least about 15%, at least about 20%, at least about 30%, at least about 40%, at least about 50%, at least about 60%, at least about 70%, at least about 75%, at least about 80%, at least about 85%, at least about 90%, at least about 95%, at least about 99%, or 100% amino acid sequence identity to a wild-type endonuclease, e.g., Cpf1, MAD7, Cas9, and/or any other endonuclease of the disclosure. In some embodiments, the endonuclease comprises about or at least about 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids. In some embodiments, the endonuclease comprises at most about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids. In some embodiments, the endonuclease comprises at least about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a HNH nuclease domain of the endonuclease. In some embodiments, the endonuclease comprises at most about: 70, 75, 80, 85, 95, 97, 99, or 100% identity to a wild-type endonuclease (e.g., Cpf1, MAD7, Cas9, and/or any other endonuclease of the disclosure) over about or at least about 10 contiguous amino acids in a HNH nuclease domain of the endonuclease. In some embodiments, the endonuclease comprises at least about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a RuvC nuclease domain of the endonuclease. In some embodiments, the endonuclease comprises at most about: 70, 75, 80, 85, 90, 95, 97, 99, or 100% identity to a wild-type endonuclease (e.g., Cpf1, MAD7, Cas9, and/or any other endonuclease of the disclosure) over about or at least about 10 contiguous amino acids in a RuvC nuclease domain of the endonuclease. The present disclosure provides a guide RNAs (gRNAs) that can direct the activities of an associated endonuclease to a specific target site within a polynucleotide. In some embodiments, a guide RNA comprises a spacer sequence that hybridizes to a target nucleic acid sequence of interest, and a CRISPR repeat sequence. In some embodiments, for example in CRISPR Type II systems, the gRNA also comprises a second RNA called the tracrRNA sequence. In some embodiments, in the CRISPR Type II guide RNA (gRNA), the CRISPR repeat sequence and tracrRNA sequence hybridize to each other to form a duplex. In some embodiments, in CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease.

In some embodiments, a tracrRNA sequence comprises nucleotides that hybridize to a CRISPR repeat sequence in a cell. A tracrRNA sequence and a CRISPR repeat sequence may form a duplex, i.e., a base-paired double-stranded structure. Together, the tracrRNA sequence and the CRISPR repeat can bind to an RNA-guided endonuclease. In some embodiments, at least a part of the tracrRNA sequence can hybridize to the CRISPR repeat sequence. In some embodiments, the tracrRNA sequence can be at least about 30%, about 40%, about 50%, about 60%, about 65%, about 70%, about 75%, about 80%, about 85%, about 90%, about 95%, or 100% complementary to the CRISPR repeat sequence. In some embodiments, a tracrRNA sequence can have a length from about 7 nucleotides to about 100 nucleotides. For example, the tracrRNA sequence can be from about 7 nucleotides (NTs) to about 50 NTs, from about 7 NTs to about 40 NTs, from about 7 NTs to about 30 NTs, from about 7 NTs to about 25 NTs, from about 7 NTs to about 20 NTs, from about 7 NTs to about 15 NTs, from about 8 NTs to about 40 NTs, from about 8 NTs to about 30 NTs, from about 8 NTs to about 25 NTs, from about 8 NTs to about 20 NTs, from about 8 NTs to about 15 NTs, from about 15 NTs to about 100 NTs, from about 15 NTs to about 80 NTs, from about 15 NTs to about 50 NTs, from about 15 NTs to about 40 NTs, from about 15 NTs to about 30 NTs or from about 15 NTs to about 25 NTs long. In some embodiments, the tracrRNA sequence can be approximately 9 nucleotides in length. In some embodiments, the tracrRNA sequence can be approximately 12 nucleotides.

In some embodiments, the tracrRNA sequence can be at least about 60% identical to a reference tracrRNA (e.g., wild type, tracrRNA from S. pyogenes) sequence over a stretch of at least 6, 7, or 8 contiguous nucleotides. For example, a tracrRNA sequence can be at least about 65% identical, about 70% identical, about 75% identical, about 80% identical, about 85% identical, about 90% identical, about 95% identical, about 98% identical, about 99% identical or 100% identical to a reference tracrRNA sequence over a stretch of at least 6, 7, or 8 contiguous nucleotides.

In some embodiments, the Cas protein or the endonuclease can be introduced into a cell containing the target polynucleotide sequence in the form of a nucleic acid encoding the Cas protein or the endonuclease (e.g., Cas9, Cpf1, MAD7, or any endonuclease or Cas protein of the disclosure). In some embodiments, the method includes a technique to introduce a nucleic acid into γδ iPSC cells. The process of introducing the nucleic acids into cells can be achieved by any suitable technique. Suitable techniques include, but are not limited to, transfection (e.g., neon transfection, calcium phosphate or lipid-mediated transfection), electroporation, and transduction or infection using a viral vector. In some embodiments transfection is performed using Neon transfection. In some embodiments, transfection is performed with Lonza Nucleofection. In some embodiments, nucleic acids are introduced into cells using a non-viral system (e.g., neon transfection). In some embodiments, nucleic acids are introduced into cells using a viral system (e.g., adenoassociated virus). In some embodiments, the method includes electroporation of a γδ iPSC cell to introduce genetic material including, for example, DNA, RNA, and/or mRNA. In some embodiments a technique to introduce a protein or nucleic acid can include introducing a protein or nucleic acid via electroporation; microinjection; viral delivery; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with a endosomolytic agent; use of cell-penetrating peptides; or any other suitable technique. In some embodiments, the method includes electroporation of a γδ iPSC cell including, for example, using a Neon transfection system (Thermo Fisher Scientific Inc.).

In some embodiments, the nucleic acid comprises DNA. In some embodiments, the nucleic acid comprises a modified DNA. In some embodiments, the nucleic acid comprises mRNA. In some embodiments, the nucleic acid comprises a modified mRNA.

In some embodiments, the Cas protein or endonuclease is complexed with at least one ribonucleic acid (e.g., one to two ribonucleic acid(s)). In some embodiments, the Cas protein or endonuclease is complexed with two ribonucleic acids. In some embodiments, the Cas protein or endonuclease is complexed with one ribonucleic acid. In some embodiments, the Cas protein or endonuclease is encoded by a modified nucleic acid.

In some embodiments, the endonuclease and gRNA can each be administered separately to a cell. In some embodiments, the endonuclease can be pre-complexed with one or more guide RNAs, or one or more crRNA together with a tracrRNA. The pre-complexed material can then be administered to a cell. Such pre-complexed material is known as a ribonucleoprotein particle (RNP). The endonuclease in the RNP can be, for example, a Cpf1 endonuclease, a MAD7 endonuclease, a Cas9 endonuclease, or any endonuclease of the disclosure. In some aspects, the RNA-guided endonuclease is Acidaminococcus sp. BV3L6 Cas12a (Cpf1). In some embodiments, the endonuclease can be flanked at the N-terminus, the C-terminus, or both the N-terminus and C-terminus by one or more nuclear localization signals (NLSs). In some embodiments, the weight ratio of genome-targeting nucleic acid to endonuclease in the RNP can be 1:1, 2:1, 1:2, or any suitable ratio.

In some embodiments, the gRNA can be a double-molecule guide RNA. In some embodiments, the gRNA can be a single-molecule guide RNA (sgRNA).

In some embodiments, a gRNA comprises a sequence that hybridizes to a sequence in a target polynucleotide. In some embodiments, the nucleotide sequence of the gRNA can vary depending on the sequence of the target nucleic acid of interest. In some embodiments, a gRNA comprises a variable length sequence with 17-30 nucleotides, in which at least a portion of the sequence hybridizes to a sequence in a target polynucleotide. In some embodiments, a gRNA sequence can be designed to hybridize to a target polynucleotide that is located 5′ of a PAM of the endonuclease used in the system.

In some embodiments, a gRNA comprises another moiety (e.g., a stability control sequence, an endoribonuclease binding sequence, or a ribozyme). The moiety can decrease or increase the stability of a nucleic acid targeting nucleic acid. In some embodiments, the moiety can be a transcriptional terminator segment (i.e., a transcription termination sequence). In some embodiments, the moiety can function in a eukaryotic cell. The moiety can function in a prokaryotic cell. In some embodiments, the moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

In some embodiments, the portion of the gRNA that hybridizes to a sequence or a target motif in a target polynucleotide is referred to as a spacer. In some embodiments, the portion of the gRNA that hybridizes to a sequence or a target motif in a target polynucleotide (spacer) comprises about 15, 16, 17, 18, 19, 20, 21, 22, 23, 24, 25, or more than about 25 nucleotides. In some embodiments, the portion of the gRNA that hybridizes to a sequence or a target motif in a target polynucleotide comprises less than about 25 nucleotides. In some embodiments, the portion of the gRNA that hybridizes to a sequence or a target motif in a target polynucleotide, or the gRNA comprises more than about 20 nucleotides. In some embodiments, the portion of the gRNA that hybridizes to a sequence or a target motif in a target polynucleotide, or the gRNA comprises about or at least about: 5, 10, 15, 16, 17, 18, 19, 20, 21, 22, 23, 24, 25, 30, 35, 40, 45, 50 or more nucleotides. In some embodiments, the portion of the gRNA that hybridizes to a sequence or a target motif in a target polynucleotide, or the gRNA comprises at most about: 5, 10, 15, 16, 17, 18, 19, 20, 21, 22, 23, 24, 25, 30, 35, 40, 45, 50 or more nucleotides. In some embodiments, the sequence or target motif in a target polynucleotide sequence comprises about, at least about, or at most about 20 bases immediately 5′ of the first nucleotide of the PAM.

In some embodiments, the portion of the gRNA that hybridizes to a sequence or a target motif in a target polynucleotide has a length of at least about 6 nucleotides (NTs). In some embodiments, the portion of the gRNA that hybridizes to a sequence or a target motif in a target polynucleotide, or the gRNA is about or at least about 6 NTs, about or at least about 10 NTs, about or at least about 15 NTs, about or at least about 18 NTs, about or at least about 19 NTs, about or at least about 20 NTs, about or at least about 21 NTs, about or at least about 22 NTs, about or at least about 23 NTs, about or at least about 24 NTs, about or at least about 25 NTs, about or at least about 30 NTs, about or at least about 35 NTs, about or at least about 40 NTs, about or at least about 45 NTs, about or at least about 50 NTs, or more than about 50 NTs. In some embodiments, the portion of the gRNA that hybridizes to a sequence or a target motif in a target polynucleotide, or the gRNA is from about 6 NTs to about 40 NTs, from about 6 NTs to about 35 NTs, from about 6 NTs to about 30 NTs, from about 6 NTs to about 29 NTs, from about 6 NTs to about 28 NTs, from about 6 NTs to about 27 NTs, from about 6 NTs to about 26 NTs, from about 6 NTs to about 25 NTs, from about 6 NTs to about 24 NTs, from about 6 NTs to about 23 NTs, from about 6 NTs to about 22 NTs, from about 6 NTs to about 21 NTs, from about 6 NTs to about 20 NTs, from about 10 NTs to about 50 NTs, from about 10 NTs to about 40 NTs, from about 10 NTs to about 35 NTs, from about 10 NTs to about 30 NTs, from about 10 NTs to about 30 NTs, from about 10 NTs to about 29 NTs, from about 10 NTs to about 28 NTs, from about 10 NTs to about 27 NTs, from about 10 NTs to about 26 NTs, from about 10 NTs to about 25 NTs, from about 10 NTs to about 24 NTs, from about 10 NTs to about 23 NTs, from about 10 NTs to about 22 NTs, from about 10 NTs to about 21 NTs, from about 10 NTs to about 20 NTs, from about 19 NTs to about 23 NTs, from about 19 NTs to about 24 NTs, from about 19 NTs to about 25 NTs, from about 19 NTs to about 30 NTs, from about 19 NTs to about 35 NTs, from about 19 NTs to about 40 NTs, from about 19 NTs to about 45 NTs, from about 19 NTs to about 50 NTs, from about 19 NTs to about 60 NTs, from about 20 NTs to about 25 NTs, from about 20 NTs to about 30 NTs, from about 20 NTs to about 35 NTs, from about 20 NTs to about 40 NTs, from about 20 NTs to about 45 NTs, from about 20 NTs to about 50 NTs, or from about 20 NTs to about 60 NTs.

In some embodiments, the percent complementarity between the gRNA or a portion of the gRNA (e.g., spacer or crRNA) and the target polynucleotide is about or at least about 30%, about or at least about 40%, about or at least about 50%, about or at least about 60%, about or at least about 65%, about or at least about 70%, about or at least about 75%, about or at least about 80%, about or at least about 85%, about or at least about 90%, about or at least about 95%, about or at least about 97%, about or at least about 98%, about or at least about 99%, or 100%. In some embodiments, the percent complementarity between the gRNA or a portion of the gRNA and the target polynucleotide is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the length of the portion of the gRNA and the target nucleic acid can differ by 1 to 6 nucleotides, which may be thought of as a bulge or bulges.

In some embodiments, the gRNA or a portion of the gRNA (e.g., a spacer or crRNA) comprises any one of SEQ ID NOs: 7-11 or 18 or portions or fragments thereof. In some embodiments, the gRNA or a portion of the gRNA (e.g., a spacer or crRNA) consists of any one of SEQ ID NOs: 7-11 or 18 or portions or fragments thereof. In some embodiments, the gRNA or a portion of the gRNA (e.g., a spacer or crRNA) comprises or consists of at least one nucleotide modification relative to any one of SEQ ID NOs: 7-11 or 18. In some embodiments, the gRNA or a portion of the gRNA (e.g., a spacer or crRNA) comprises or consists of about 1, 2, 3, 4, 5, or more than 5 nucleotide modification(s) relative to any one of SEQ ID NOs: 7-11 or 18. In some embodiments, the gRNA or a portion of the gRNA (e.g., a spacer or crRNA) is about, at least about, or at most about 75%, 80%, 85%, 90%, 91%, 92%, 93%, 94%, 95%, 96%, 97%, 98%, 99%, or 100% identical to any one of SEQ ID NOs: 7-11 or 18 or portions or fragments thereof. In some embodiments, the gRNA or a portion of the gRNA (e.g., a spacer or crRNA) is about, at least about, or at most about 75%, 80%, 85%, 90%, 91%, 92%, 93%, 94%, 95%, 96%, 97%, 98%, 99%, or 100% identical to about or at least about 10, 11, 12, 13, 14, 15, 16, 17, 18, 19, 20, 21, 22, 23, or 24 contiguous nucleotides of any one of SEQ ID NOs: 7-11 or 18. In some embodiments, the gRNA or a portion of the gRNA (e.g., a spacer or crRNA) comprises a sequence comprising at least one, two, three, four, five, or more than five nucleotide mismatches compared to any one of SEQ ID NOs: 7-11 or 18.

In some embodiments, a gRNA is modified or chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art.

In some embodiments, a modified gRNA comprises a modified backbone, for example, phosphorothioates, phosphotriesters, morpholinos, methyl phosphonates, short chain alkyl or cycloalkyl intersugar linkages or short chain heteroatomic or heterocyclic intersugar linkages.

In some embodiments, a modified gRNA comprises one or more substituted sugar moieties, e.g., one of the following at the 2′ position: OH, SH, SCH₃, F, OCN, OCH₃, OCH₃O(CH₂)n CH₃, O(CH₂)n NH₂, or O(CH₂)n CH₃, where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH 3); 2′-propoxy (2′-OCH₂CH₂CH₃); and 2′-fluoro (2′-F). Similar modifications may also be made at other positions on the gRNA, for example, the 3′ position of the sugar on the 3′ terminal nucleotide and/or the 5′ position of 5′ terminal nucleotide. In some examples, both a sugar and an internucleoside linkage, i.e., the backbone, of the nucleotide units can be replaced with different groups.

In some embodiments, a gRNA includes, additionally or alternatively, nucleobase (or “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, 5-methylcytosine (also referred to as 5-methyl-2′ deoxycytosine or 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine.

In some embodiments, modified nucleobases can include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, and 3-deazaguanine and 3-deazaadenine.

In some embodiments, methods of modifying a gene or genome editing of the disclosure can be performed using a zinc finger nuclease (ZFN). Zinc finger nucleases (ZFNs) are modular proteins comprised of an engineered zinc finger DNA binding domain linked to the catalytic domain of the type II endonuclease Fokl. Because Fokl functions as a dimer, a pair of ZFNs is engineered to bind to cognate target “half-site” sequences on opposite DNA strands and with precise spacing between them to enable the catalytically active Fokl dimer to form. Upon dimerization of the Fokl domain, a DNA double-strand break is generated between the ZFN half-sites as the initiating step in genome editing.

In some embodiments, the DNA binding domain of each ZFN is comprised of 3-6 zinc fingers of the abundant Cys2-His2 architecture, with each finger primarily recognizing a triplet of nucleotides on one strand of the target DNA sequence, although cross-strand interaction with a fourth nucleotide can also occur. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. ZFNs can be readily re-targeted to almost any genomic address simply by modifying individual fingers. In some embodiments, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96(6):2758-63; Dreier B et al., J Mol Biol., 2000, 303(4):489-502; Liu Q et al., J Biol Chem., 2002, 277(6):3850-6; Dreier et al., J Biol Chem., 2005, 280(42):35588-97; and Dreier et al., J Biol Chem. 2001, 276(31):29466-78.

In some embodiments, methods of modifying a gene or genome editing of the disclosure can be performed using a Transcription Activator-Like Effector Nuclease (TALEN). TALEN represent another format of modular nucleases whereby, as with ZFNs, an engineered DNA binding domain is linked to the Fokl nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. A variety of TALEN-based systems have been described in the art, and modifications thereof are regularly reported; see, e.g., Boch, Science, 2009 326(5959):1509-12; Mak et al., Science, 2012, 335(6069):716-9; and Moscou et al., Science, 2009, 326(5959):1501. The use of TALENs based on the “Golden Gate” platform, or cloning scheme, has been described by multiple groups; see, e.g., Cermak et al., Nucleic Acids Res., 2011, 39(12):e82; Li et al., Nucleic Acids Res., 2011, 39(14):6315-25; Weber et al., PLoS One., 2011, 6(2):e16765; Wang et al., J Genet Genomics, 2014, 41(6):339-47; and Cermak T et al., Methods Mol Biol., 2015 1239:133-59.

In some embodiments, methods of modifying a gene or genome editing of the disclosure can be performed using a Homing Endonuclease (HE). Homing endonucleases (HEs) are sequence-specific endonucleases that have long recognition sequences (14-44 base pairs) and cleave DNA with high specificity—often at sites unique in the genome. There are at least six known families of HEs as classified by their structure, including GIY-YIG, His-Cis box, H—N—H, PD-(D/E)xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. A variety of HE-based systems have been described in the art, and modifications thereof are regularly reported; see, e.g., the reviews by Steentoft et al., Glycobiology, 2014, 24(8):663-80; Belfort and Bonocora, Methods Mol Biol., 2014, 1123:1-26; and Hafez and Hausner, Genome, 2012, 55(8):553-69.

In some embodiments, methods of modifying a gene or genome editing of the disclosure can be performed using a MegaTAL or Tev-mTALEN platforms. The MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., Nucleic Acids Res., 2014, 42: 2591-2601; Kleinstiver et al., G3, 2014, 4:1155-65; and Boissel and Scharenberg, Methods Mol. Biol., 2015, 1239: 171-96.

In some embodiments, the MegaTev architecture is the fusion of a meganuclease (Mega) with the nuclease domain derived from the GIY-YIG homing endonuclease I-Teel (Tev). The two active sites are positioned ˜30 bp apart on a DNA substrate and generate two DSBs with non-compatible cohesive ends; see, e.g., Wolfs et al., Nucleic Acids Res., 2014, 42, 8816-29. It is anticipated that other combinations of existing nuclease-based approaches will evolve and be useful in achieving the targeted genome modifications described herein.

### 5.5. B2M Knock Out iPSCs Derived from γδ T Cells

γδ T cells-derived iPSCs express HLA class I, which is one of the most polymorphic human antigens and so frequently leads to recognition by allogenic T cells, resulting in allorejection. As a result, such iPSCs (and immunotherapeutics based on them) have limited applicability in a clinical setting, without additional interventions (such as host immune system suppression/depletion). B2M is required for surface expression of HLA-I molecules. As such, disrupting B2M results in reduced/eliminated surface expression of HLA-I molecules. The resulting iPSCs are therefore improved because they are more suitable for use in an allogenic setting (e.g. to develop so-called “off-the-shelf” therapies).

In some embodiments, a genetically modified cell of the disclosure (e.g., B2M knock out and/or HLA-A, HLA-B, and/or HLA-C knock out iPSCs derived from γδ T cells) comprises the introduction of at least one genetic modification within or near at least one gene (e.g., B2M) that results in decreased or altered expression of B2M and/or of one or more MHC-I human leukocyte antigens (e.g., HLA-A, HLA-B, and/or HLA-C) relative to an unmodified cell. In some embodiments, a genetically modified cell of the disclosure or a population thereof has reduced immunogenicity or reduced immune response as compared to an unmodified cell or a population of unmodified cells (e.g., compared to iPSCs derived from γδ T cells and that do not comprise B2M knock out and/or HLA-A, HLA-B, and/or HLA-C knock out). In some embodiments, a population of genetically modified cells of the disclosure have reduced immunogenicity or reduced immune response by about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (lower) as compared to a population of unmodified cells. In some embodiments, cells are assessed for immunogenicity using any suitable method known to a skilled artisan. In some embodiments, a cell is analyzed for the presence of antibodies on the cell surface, e.g., by staining with an anti-IgM antibody. In some embodiments, immunogenicity is assessed by a PBMC cell lysis assay. In some embodiments, a population of cell is incubated with peripheral blood mononuclear cells (PBMCs) and then assessed for lysis of the cells by the PBMCs. In some embodiments, immunogenicity is assessed by a natural killer (NK) cell lysis assay. In some embodiments, a population of cells is incubated with NK cells and then assessed for lysis of the cells by the NK cells. In some embodiments, immunogenicity is assessed by a CD8+ T cell lysis assay. In some embodiments, a population of cells is incubated with CD8+ T cells and then assessed for lysis of the cells by the CD8+ T cells. In some embodiments, a genetically modified cell of the disclosure or a population thereof has increased viability or increased survival rate as compared to an unmodified cell or a population of unmodified cells (e.g., compared to iPSCs derived from γδ T cells and that do not comprise B2M knock out and/or HLA-A, HLA-B, and/or HLA-C knock out). In some embodiments, a population of genetically modified cells of the disclosure have increased viability or increased survival rate of about or at least about 5%, 10%, 15%, 20%, 25%, 30%, 35%, 40%, 45%, 50%, 55%, 60%, 65%, 70%, 75%, 80%, 85%, 90%, 95%, 100% or more than 100% (higher) as compared to a population of unmodified cells. In some embodiments, cells are assessed for increased viability or increased survival rate using any suitable method known to a skilled artisan. In some embodiments, cell viability or survival rate is determined using flow cytometry, high content imaging, tetrazolium reduction (MTT) assay, resazurin reduction assay, protease viability marker assay, and/or ATP detection assay.

In some embodiments, a genetically modified cell of the disclosure comprises the introduction of at least one genetic modification within or near at least one gene that decreases the expression of one or more MHC-I human leukocyte antigens relative to an unmodified cell. In some embodiments, genetically modified cells of the disclosure comprise at least one deletion of a gene or at least a partial inactivation of a gene (e.g., B2M). In some embodiments, genetically modified cells of the disclosure comprise at least one deletion within or near at least one gene that alters the expression of one or more MHC-I human leukocyte antigens relative to an unmodified cell.

In some embodiments, the genome of a cell is modified to decrease the expression of beta-2-microglobulin (B2M). HLA-I proteins are intimately associated with B2M in the endoplasmic reticulum, which is essential for forming functional, cell-surface expressed HLA-I molecules. In some embodiments, the gRNA targets a site within the B2M gene as described in Sections 5.3 and 5.4. In some embodiments, B2M expression is not detected in a population of genetically modified cells of the disclosure (e.g., not detected by a conventional method (e.g., FACS)). In some embodiments, a population of genetically modified cells of the disclosure has complete or at least partial reduced expression of B2M as compared to a population of unmodified cells. In some embodiments, the expression of B2M in a population of genetically modified cells is reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expression of B2M in a population of unmodified cells.

In some embodiments, the genetically modified cell of the disclosure does not have a detectable level of B2M. In some embodiments, the genetically modified cell does not have a detectable RNA transcript encoding B2M. In some embodiments, the genetically modified cell does not have a detectable level of B2M protein. In some embodiments, the iPSCs do not express a detectable level of B2M protein when analysed by flow cytometry.

In some embodiments, a genetically modified cell of the disclosure comprises a genomic modification of one or more MHC-I genes. In some embodiments, a genetically modified cell of the disclosure comprises a genomic modification of one or more polynucleotide sequences that regulates the expression of MHC-I. In some embodiments, a genetic modification of the disclosure is performed using any gene editing method including but not limited to those methods described herein (see Section 5.4). The genes that encode the major histocompatibility complex (MHC) are located on human Chr. 6p21. MHC-I genes (HLA-A, HLA-B, and HLA-C) are expressed in almost all tissue cell types, presenting “non-self” antigen-processed peptides to CD8+ T cells, thereby promoting their activation to cytolytic CD8+ T cells. MHC-I proteins are intimately associated with beta-2-microglobulin (B2M) in the endoplasmic reticulum, which is essential for forming functional MHC-I molecules on the cell surface.

In some embodiments, decreasing the expression of one or more MHC-I human leukocyte antigens relative to an unmodified cell is accomplished by targeting, e.g., for genetic deletion at least one base pair, in a MHC-I gene directly. In some embodiments, decreasing the expression of one or more MHC-I human leukocyte antigens relative to an unmodified cell is accomplished by targeting, e.g., for genetic deletion, a B2M gene. In some embodiments, decreasing the expression of one or more MHC-I human leukocyte antigens relative to an unmodified cell is accomplished by targeting, e.g., for genetic deletion, at least one transcriptional regulator of MHC-I. In some embodiments, the genome of a cell is modified to delete the entirety or a portion of a HLA-A, HLA-B, and/or HLA-C gene. In some embodiments, the genome of a cell is modified to delete the entirety or a portion of a promoter region of a HLA-A, HLA-B, and/or HLA-C gene. In some embodiments, the genome of a cell is modified to delete the entirety or a portion of a gene that encodes a transcriptional regulator of MHC-I. In some embodiments, the genome of a cell is modified to delete the entirety or a portion of a promoter region of a gene that encodes a transcriptional regulator of MHC-I.

In some embodiments, HLA-A, HLA-B, and/or HLA-C expression is not detected in a population of genetically modified cells of the disclosure (e.g., not detected by a conventional method (e.g., FACS)). In some embodiments, a population of genetically modified cells of the disclosure has complete or at least partial reduced expression of HLA-A, HLA-B, and/or HLA-C as compared to a population of unmodified cells. In some embodiments, the expression of HLA-A in a population of genetically modified cells is reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expression of HLA-A in a population of unmodified cells. In some embodiments, the expression of HLA-B in a population of genetically modified cells is reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expression of HLA-B in a population of unmodified cells. In some embodiments, the expression of HLA-C in a population of genetically modified cells is reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expression of HLA-C in a population of unmodified cells. In some embodiments, the expression of B2M, HLA-A, HLA-B, and/or HLA-C in a population of genetically modified cells is reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expression of B2M, HLA-A, HLA-B, and/or HLA-C in a population of unmodified cells. In some embodiments, the expressions of B2M, HLA-A, HLA-B, and HLA-C in a population of genetically modified cells are reduced by about or at least about 5%, 10%, 15%, 20%, 25%, 30%, 35%, 40%, 45%, 50%, 55%, 60%, 65%, 70%, 75%, 80%, 85%, 90%, 95%, 100% (lower) as compared to the expressions of B2M, HLA-A, HLA-B, and HLA-C in a population of unmodified cells. In some embodiments, expressions of B2M, HLA-A, HLA-B, and HLA-C is detected by FACS.

### 5.6. Methods of Producing Induced Pluripotent Stem Cells (iPSCs)

In some embodiments, the iPSCs of the disclosure and methods of making the same is disclosed in WO2021/257679 (PCT/US2021/037594), which is herein incorporated by reference in its entirety. In one aspect, provided herein are methods for reprogramming somatic cells (e.g., a T cell) to a less differentiated state. The resulting cells are called reprogrammed somatic cells herein. A reprogrammed somatic cell may be a reprogrammed somatic cell of varying differentiation status. In some embodiments, the reprogrammed somatic cell is an induced pluripotent stem cell (iPSC). The present disclosure is based in part on the surprising discovery that a combination of various factors, e.g., a combination of zoledronic acid and Interleukin-15 (IL-15), can activate γδ T cells and thus improve the efficiency of induction of pluripotency in non-pluripotent mammalian T cells transformed with transcription factors. Accordingly, in one aspect, the present disclosure provides for methods of inducing pluripotency in non-pluripotent mammalian γδ T cells (e.g., Vγ9⁺γδ T cells) wherein the method comprises contacting peripheral blood mononuclear cells (PBMCs) with an activation culture comprising IL-15 and zoledronic acid.

In some embodiments, the iPSCs of the disclosure and methods of making the same is disclosed in WO2021/176373 (PCT/M2021/051779), which is herein incorporated by reference in its entirety. In some embodiments, provided herein is a method of producing induced pluripotent stem cells (iPSCs), comprising: (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with a viral vector encoding one or more reprogramming factors; (d) culturing the transduced γδ T cells under conditions suitable for reprogramming mammalian somatic cells to a pluripotent state, thereby producing a population of iPSCs; (e) contacting the population of iPSCs with an RNA-guided endonuclease or a nucleic acid encoding the RNA-guided endonuclease and a guide RNA (gRNA). In some embodiments, the gRNA binds to a target motif of a beta-2-microglobulin (B2M) polynucleotide sequence in the population of iPSCs. In some embodiments, the contacting results in cleavage of the B2M polynucleotide sequence.

In some embodiments, provided herein are methods of reprogramming a somatic cell comprising: (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with one or more viral vector(s) encoding one or more reprogramming factors; and (d) culturing the transduced γδ T cells under conditions suitable for reprogramming mammalian somatic cells to a less differentiated state. In some embodiments, the less differentiated state is a multipotent state. In some embodiments, the less differentiated state is a pluripotent state.

In some more specific embodiments, provided herein are methods of producing induced pluripotent stem cells (iPSCs) comprising: (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with one or more viral vector(s) encoding one or more reprogramming factors; and (d) culturing the transduced γδ T cells under conditions suitable for reprogramming mammalian somatic cells to a pluripotent state.

In certain embodiments, the activation culture further comprises one or more additional agents or compounds, for example, to improve the efficiency of activation or induction. In one embodiment, the activation culture further comprises Interleukin-2 (IL-2).

Therefore, in some embodiments, provided herein are methods of reprogramming a somatic cell comprising: (a) contacting an isolated population of cells with an activation culture; wherein the activation culture comprises IL-15, zoledronic acid and IL-2; (b) culturing the isolated population of cells in the activation culture to enrich and/or activate γδ T cells in the isolated population of cells; (c) transducing the γδ T cells with one or more viral vector(s) encoding one or more reprogramming factors; and (d) culturing the transduced γδ T cells under conditions suitable for reprogramming mammalian somatic cells to a less differentiated state.

In some more specific embodiments, provided herein are methods of producing induced pluripotent stem cells (iPSCs) comprising: (a) contacting an isolated population of cells with an activation culture; wherein the activation culture comprises IL-15, zoledronic acid and IL-2; (b) culturing the isolated population of cells in the activation culture to enrich and/or activate γδ T cells in the isolated population of cells; (c) transducing the γδ T cells with one or more viral vector(s) encoding one or more reprogramming factors; and (d) culturing the transduced γδ T cells under conditions suitable for reprogramming mammalian somatic cells to a pluripotent state.

In some embodiments, the method further comprises delivery of a donor template comprising a knock-in cassette comprising a cargo sequence for insertion within the target gene. In some embodiments, endonucleases are introduced to edit and insert knock-in genes into the B2M gene. In some embodiments, endonucleases comprise zinc-finger nucleases (ZFN), transcription activator-like effector nucleases (TALEN), or endonuclease of the CRISPR/CAS systems.

In some embodiments, transposases, recombinases, or integrases are introduced to insert knock-in genes into the B2M gene. In some embodiments, transposases, recombinases, or integrases are conjugated to a targeting moiety such as a zinc finger, transcription activator-like effector (TALE), or nuclease-deficient CRISPR/CAS molecule. In some embodiments, introduction of a cargo encoding construct includes the introduction of a vector DNA and a transposase. In some embodiments, a zinc finger nuclease is used as a gene editing agent in any one of the embodiments described herein. Particular embodiments may use a transcription activator like nuclease (TALEN) as a gene editing agent. In some embodiments, targeted genetic engineering approaches can be used, such as the CRISPR nuclease system.

One of skill in the art would appreciate that transposon systems may be used to insert a cassette encoding a cargo into a B2M gene. In some embodiments, a Sleeping Beauty transposon system is used to insert an expression cassette into a B2M gene, wherein the expression cassette encodes a transgene. In some embodiments, a MOS1 transposase system is used to insert an expression cassette into a B2M gene, wherein the expression cassette encodes a transgene. In some embodiments herein, a PiggyBac transposase is used to insert an expression cassette into a B2M gene, wherein the expression cassette encodes a transgene. In some embodiments, a ISY100 transposase is used to insert an expression cassette into a B2M gene, wherein the expression cassette encodes a transgene. In some embodiments, the expression cassette further comprises transcriptional regulation elements. In some embodiment, the transposases described herein further comprise a fusion protein comprising a nuclease-dead Cas protein, TALE or zinc finger protein. In some embodiments, the transgene encodes a therapeutic cargo, a protein, a TCR or a chimeric antigen receptor.

In some embodiments, the method further comprises creating DSBs within the B2M gene and inserting a nucleic acid construct within or near the target gene. In some embodiments, the double strand breaks are performed by endonucleases and the polynucleotide is inserted via HDR. In some embodiments, the method further comprises providing a nucleic acid for insertion within a target gene. In some embodiments, the nucleic acid is a vector encoding a cargo. In some embodiments, the cargo comprises a therapeutic protein. In some embodiments, the nucleic acid comprises a gene encoding a therapeutic protein. In some embodiments, the nucleic acid comprises a gene encoding a TCR. In some embodiments, the nucleic acid encodes a CAR construct (CAR) for insertion within a target gene. In some embodiments, the target is within the B2M gene. In some embodiments, the target is within SEQ ID NO: 1. In some embodiments, the CAR comprises an ectodomain comprising an antigen recognition region, a transmembrane domain, and an endodomain comprising at least one costimulatory domain. In some embodiments, the nucleic acid further comprises a promoter, at least one gene regulatory elements, or a combination thereof.

In certain embodiments, the method further comprises obtaining the isolated population of cells from a subject. In certain embodiments, the subject is a mammal. In certain embodiments, the subject is a human.

In certain embodiments, the isolated population of cells are peripheral blood cells, cord blood cells, or bone marrow cells. In one embodiment, the isolated population of cells are peripheral blood mononuclear cells (PBMCs).

Methods for identifying reprogrammed mammalian somatic cells with a less differentiated state or a pluripotent state are known in the art. For example, in some embodiments, reprogrammed somatic cells are identified by selecting for cells that express the appropriate selectable marker. In some embodiments, reprogrammed somatic cells are further assessed for pluripotency characteristics. The presence of pluripotency characteristics indicates that the somatic cells have been reprogrammed to a pluripotent state.

Differentiation status of cells is a continuous spectrum, with terminally differentiated state at one end of this spectrum and de-differentiated state (pluripotent state) at the other end. Reprogramming, as used herein, refers to a process that alters or reverses the differentiation status of a somatic cell, which can be either partially or terminally differentiated. Reprogramming includes complete reversion, as well as partial reversion, of the differentiation status of a somatic cell. In other words, the term “reprogramming,” as used herein, encompasses any movement of the differentiation status of a cell along the spectrum toward a less-differentiated state. For example, reprogramming includes reversing a multipotent cell back to a pluripotent cell, reversing a terminally differentiated cell back to either a multipotent cell or a pluripotent cell. In one embodiment, reprogramming of a somatic cell turns the somatic cell all the way back to a pluripotent state. In another embodiment, reprogramming of a somatic cell turns the somatic cell back to a multipotent state. The term “less-differentiated state,” as used herein, is thus a relative term and includes a completely de-differentiated state and a partially differentiated state.

The term “pluripotency characteristics” refers to many characteristics associated with pluripotency, including, for example, the ability to differentiate into all types of cells and an expression pattern distinct for a pluripotent cell, including expression of pluripotency genes, expression of other ES cell markers, and on a global level, a distinct expression profile known as “stem cell molecular signature” or “stemness.”

Thus, to assess reprogrammed somatic cells for pluripotency characteristics, one may analyze such cells for different growth characteristics and ES cell-like morphology. In some embodiments, cells may be injected subcutaneously into immunocompromised SCID mice to induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another pluripotency characteristics that can be monitored.

In some embodiments, functional assays of the reprogrammed somatic cells may be conducted by introducing them into blastocysts to determine whether the cells are capable of giving rise to all cell types. If the reprogrammed cells are capable of forming a few cell types of the body, they are multipotent; if the reprogrammed cells are capable of forming all cell types of the body including germ cells, they are pluripotent.

In other embodiments, the expression of an individual pluripotency gene in the reprogrammed somatic cells may be examined to assess their pluripotency characteristics.

Additionally, one may assess the expression of other ES cell markers. Stage-specific embryonic 1 5 antigens-1, -3, and -4 (SSEA-1, SSEA-3, SSEA-4) are glycoproteins specifically expressed in early embryonic development and are markers for ES cells (Solter and Knowles, 1978, Proc. Natl. Acad. Sci. USA 75:5565-5569; Kannagi et al., 1983, EMBO J 2:2355-2361).

Elevated expression of the enzyme Alkaline Phosphatase (AP) is another marker associated with undifferentiated embryonic stem cells (Wobus et al., 1984, Exp. Cell 152:212-219; Pease et al., 1990, Dev. Biol. 141:322-352). Other stem/progenitor cells markers include the intermediate neurofilament nestin (Lendahl et al., 1990, Cell 60:585-595; Dah-Istrand et al., 1992, J. Cell Sci. 103:589-597), the membrane glycoprotein prominin/AC133 (Weigmann et al., 1997, Proc. Natl. Acad. USA 94:12425-12430; Corbeil et al., 1998, Blood 91:2625-22626), the transcription factor Tcf-4 (Korinek et al, 1998, Nat. Genet. 19: 379-383; Lee et al., 1999, J. Biol. Chem. 274.1566-1572), and the transcription factor Cdx1 (Duprey et al., 1988, Genes Dev. 2:1647-1654; Subramania'n et al., 1998, Differentiation 64:11-18).

In some embodiments, expression profiling of the reprogrammed somatic cells may be used to assess their pluripotency characteristics. Pluripotent cells, such as embryonic stem cells, and multipotent cells, such as adult stem cells, are known to have a distinct pattern of global gene expression profile. This distinct pattern is termed “stem cell molecular signature”, or “stemness”. See, for example, Ramalho-Santos et al., Science 298: 597-600 (2002); Ivanova et al., Science 298: 601-604.

Somatic cells may be reprogrammed to gain either a complete set of the pluripotency characteristics and are thus pluripotent. Alternatively, somatic cells may be reprogrammed to gain only a subset of the pluripotency characteristics. In another alternative, somatic cells may be reprogrammed to be multipotent.

Activation Culturing

In certain embodiments, the isolated population of cells are cultured in the activation culture for a first period of time. In certain embodiments, the first period of time is 1-20 days. In certain embodiments, the first period of time is 1-17 days. In certain embodiments, the first period of time is 1-15 days. In certain embodiments, the first period of time is 1-13 days. In certain embodiments, the first period of time is 1-11 days. In certain embodiments, the first period of time is 1-9 days. In certain embodiments, the first period of time is 1-7 days. In certain embodiments, the first period of time is 1-5 days. In certain embodiments, the first period of time is 1-3 days. In certain embodiments, the first period of time 12-72 hours. In certain embodiments, the first period of time 12-60 hours. In certain embodiments, the first period of time 12-48 hours. In certain embodiments, the first period of time 12-36 hours. In certain embodiments, the first period of time 12-24 hours. In certain embodiments, the first period of time 8-16 hours. In certain embodiments, the first period of time 4-8 hours. In certain embodiments, the first period of time 2-4 hours. In certain embodiments, the first period of time 4-8 hours. In certain embodiments, the first period of time 50-80 hours. In certain embodiments, the first period of time 4-8 hours. In certain embodiments, the first period of time 55-75 hours. In certain embodiments, the first period of time 4-8 hours. In certain embodiments, the first period of time 60-75 hours. In certain embodiments, the first period of time 4-8 hours. In certain embodiments, the first period of time 70-75 hours. In certain embodiments, the first period of time is 1, 2, 3, 4, 5, 6, 7, 8, 9, 10, 11, 12, 13, 14, 15, 16, 17, 18, 19, or 20 days.

In certain embodiments, the isolated population of cells are cultured in the activation culture not longer than a certain period of time. For example, the isolated population of cells are cultured in the activation culture for at most 13 days, at most 12 days, at most 11 day, at most 10 days, at most 9 days, at most 8 days, at most 7 days, at most 6 days, at most 5 days, at most 4 days, at most 3 days, at most 2 days, or at most 1 day. In a specific embodiment, the isolated population of cells are cultured in the activation culture for at most 5 days. In a specific preferred embodiment, the isolated population of cells are cultured in the activation culture for at most 3 days. In a specific preferred embodiment, the isolated population of cells are cultured in the activation culture for about 3 days.

In certain embodiments, after being cultured in the activation culture for the first period of time, the isolated population of cells comprise 5%-100% γδ T cells. In certain embodiments, after being cultured in the activation culture for the first period of time, the isolated population of cells comprise 5%-95% γδ T cells. In certain embodiments, after being cultured in the activation culture for the first period of time, the isolated population of cells comprise 5%-90% γδ T cells. In certain embodiments, after being cultured in the activation culture for the first period of time, the isolated population of cells comprise 5%-85% γδ T cells. In certain embodiments, after being cultured in the activation culture for the first period of time, the isolated population of cells comprise 5%-80% γδ T cells. In certain embodiments, after being cultured in the activation culture for the first period of time, the isolated population of cells comprise 5%-75% γδ T cells. In certain embodiments, after being cultured in the activation culture for the first period of time, the isolated population of cells comprise 5%-70% γδ T cells. In certain embodiments, after being cultured in the activation culture for the first period of time, the isolated population of cells comprise 5%-65% γδ T cells. In certain embodiments, after being cultured in the activation culture for the first period of time, the isolated population of cells comprise 5%-60% γδ T cells. In certain embodiments, after being cultured in the activation culture for the first period of time, the isolated population of cells comprise 5%-55% γδ T cells. In certain embodiments, after being cultured in the activation culture for the first period of time, the isolated population of cells comprise 5%-50% γδ T cells. In certain embodiments, after being cultured in the activation culture for the first period of time, the isolated population of cells comprise 5%-45% γδ T cells. In certain embodiments, after being cultured in the activation culture for the first period of time, the isolated population of cells comprise 5%-40% γδ T cells. In certain embodiments, after being cultured in the activation culture for the first period of time, the isolated population of cells comprise 5%-35% γδ T cells. In certain embodiments, after being cultured in the activation culture for the first period of time, the isolated population of cells comprise 15%-35% γδ T cells. In certain embodiments, after being cultured in the activation culture for the first period of time, the isolated population of cells comprise 25%-35% γδ T cells. In certain embodiments, after being cultured in the activation culture for the first period of time, the isolated population of cells comprise 30%-35% γδ T cells. In certain embodiments, after being cultured in the activation culture for the first period of time, the isolated population of cells comprise 5%-30% γδ T cells. In certain embodiments, after being cultured in the activation culture for the first period of time, the isolated population of cells comprise 5%-25% γδ T cells. In certain embodiments, after being cultured in the activation culture for the first period of time, the isolated population of cells comprise 5%-20% γδ T cells. In certain embodiments, after being cultured in the activation culture for the first period of time, the isolated population of cells comprise 5%-15% γδ T cells.

In certain embodiments, after being cultured in the activation culture for the first period of time, the isolated population of cells comprise about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5% γδ T cells. In certain embodiments, after being cultured in the activation culture for the first period of time, the isolated population of cells comprise less than about 90%, less than about 80%, less than about 70%, less than about 60%, less than about 50%, less than about 45%, less than about 40%, less than about 35%, or less than about 30% γδ T cells.

In one embodiment, after being cultured in the activation culture for the first period of time, the isolated population of cells comprise less than about 60% γδ T cells. In another embodiment, after being cultured in the activation culture for the first period of time, the isolated population of cells comprise less than about 55% γδ T cells. In yet another embodiment, after being cultured in the activation culture for the first period of time, the isolated population of cells comprise less than about 50% γδ T cells. In yet another embodiment, after being cultured in the activation culture for the first period of time, the isolated population of cells comprise less than about 45% γδ T cells. In yet another embodiment, after being cultured in the activation culture for the first period of time, the isolated population of cells comprise less than about 40% γδ T cells. In yet another embodiment, after being cultured in the activation culture for the first period of time, the isolated population of cells comprise less than about 35% γδ T cells.

In certain embodiments, after being cultured in the activation culture for the first period of time, the γδ T cells in the isolated population of cells comprise 5%-100% TCRVγ9+ T cells. In certain embodiments, after being cultured in the activation culture for the first period of time, the γδ T cells in the isolated population of cells comprise 5%-95% TCRVγ9+ T cells. In certain embodiments, after being cultured in the activation culture for the first period of time, the γδ T cells in the isolated population of cells comprise 5%-90% TCRVγ9+ T cells. In certain embodiments, after being cultured in the activation culture for the first period of time, the γδ T cells in the isolated population of cells comprise 5%-85% TCRVγ9+ T cells. In certain embodiments, after being cultured in the activation culture for the first period of time, the γδ T cells in the isolated population of cells comprise 5%-80% TCRVγ9+ T cells. In certain embodiments, after being cultured in the activation culture for the first period of time, the γδ T cells in the isolated population of cells comprise 5%-75% TCRVγ9+ T cells. In certain embodiments, after being cultured in the activation culture for the first period of time, the γδ T cells in the isolated population of cells comprise 5%-70% TCRVγ9+ T cells. In certain embodiments, after being cultured in the activation culture for the first period of time, the γδ T cells in the isolated population of cells comprise 5%-65% TCRVγ9+ T cells. In certain embodiments, after being cultured in the activation culture for the first period of time, the γδ T cells in the isolated population of cells comprise 5%-60% TCRVγ9+ T cells. In certain embodiments, after being cultured in the activation culture for the first period of time, the γδ T cells in the isolated population of cells comprise 5%-55% TCRVγ9+ T cells. In certain embodiments, after being cultured in the activation culture for the first period of time, the γδ T cells in the isolated population of cells comprise 5%-50% TCRVγ9+ T cells. In certain embodiments, after being cultured in the activation culture for the first period of time, the γδ T cells in the isolated population of cells comprise 5%-45% TCRVγ9+ T cells. In certain embodiments, after being cultured in the activation culture for the first period of time, the γδ T cells in the isolated population of cells comprise 5%-40% TCRVγ9+ T cells. In certain embodiments, after being cultured in the activation culture for the first period of time, the γδ T cells in the isolated population of cells comprise 5%-35% TCRVγ9+ T cells. In certain embodiments, after being cultured in the activation culture for the first period of time, the γδ T cells in the isolated population of cells comprise 5%-30% TCRVγ9+ T cells. In certain embodiments, after being cultured in the activation culture for the first period of time, the γδ T cells in the isolated population of cells comprise 5%-25% TCRVγ9+ T cells. In certain embodiments, after being cultured in the activation culture for the first period of time, the γδ T cells in the isolated population of cells comprise 5%-20% TCRVγ9+ T cells. In certain embodiments, after being cultured in the activation culture for the first period of time, the γδ T cells in the isolated population of cells comprise 5%-15% TCRVγ9+ T cells.

In certain embodiments, after being cultured in the activation culture for the first period of time, the γδ T cells in the isolated population of cells comprise about 100%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5% TCRVγ9⁺ T cells (aka Vγ9⁺ T cells). In certain embodiments, after being cultured in the activation culture for the first period of time, the γδ T cells in the isolated population of cells comprise less than about 90%, less than about 80%, less than about 70%, less than about 60%, less than about 50%, less than about 45%, less than about 40%, less than about 35%, or less than about 30% TCRVγ9⁺ T cells.

In one embodiment, after being cultured in the activation culture for the first period of time, the γδ T cells in the isolated population of cells comprise less than about 60% TCRVγ9+ T cells. In one embodiment, after being cultured in the activation culture for the first period of time, the γδ T cells in the isolated population of cells comprise less than about 55% TCRVγ9+ T cells. In yet another embodiment, after being cultured in the activation culture for the first period of time, the γδ T cells in the isolated population of cells comprise less than about 50% TCRVγ9+ T cells. In yet another embodiment, after being cultured in the activation culture for the first period of time, the γδ T cells in the isolated population of cells comprise less than about 45% TCRVγ9+ T cells. In yet another embodiment, after being cultured in the activation culture for the first period of time, the γδ T cells in the isolated population of cells comprise less than about 40% TCRVγ9+ T cells. In yet another embodiment, after being cultured in the activation culture for the first period of time, the γδ T cells in the isolated population of cells comprise less than about 35% TCRVγ9+ T cells.

In certain embodiments, the method further comprises enriching the γδ T cells in the isolated population of cells. In certain embodiments, the γδ T cells are enriched by cell-cell clump enrichment.

In certain embodiments, at least part of the activated γδ T cells in step (b) are Vγ9⁺ γδ T cells.

In certain embodiments, at least part of the activated γδ T cells in step (b) are Vγ9δ2⁺ γδ T cells.

Cells

The present disclosure is predicated on the discovery that an isolated population of cells, e.g., an isolated population of γδ T cells, can be activated and reprogramed to pluripotency by activation (e.g., in presence of zoledronic acid and IL-15) and introduction of transcription factors (e.g., by Sendai virus vector).

The isolated population of cells of the present disclosure include any T cell of the body that is not a stem cell, a germ cell, or an iPSC. Non-limiting examples of a non-iPSC is a T cell derived from any tissue of the body, including internal organs, skin, bones, blood, nervous tissue, and connective tissue.

In certain embodiments, the isolated population of cells are blood cells. In certain embodiments, the blood cells are suitably peripheral blood mononuclear cells (PMBCs), and may include all types of blood cells existing on an entire differentiation process from hematopoietic stem cells to final differentiation into peripheral blood. In one embodiment, the blood cells include, for example, hematopoietic stem cells, lymphoid stem cells, lymphoid dendritic cell progenitor cells, lymphoid dendritic cells, T lymphocyte progenitor cells, T cells, B lymphocyte progenitor cells, B cells, plasma cells, NK progenitor cells, NK cells, monocytes, and macrophages.

In some embodiments, the isolated population of cells can be peripheral blood mononuclear cells (PBMC), peripheral blood leukocytes (PBL), tumor infiltrating lymphocytes (TIL), or a combination thereof. In some embodiments, the isolated population of cells are peripheral blood mononuclear (PBMC) cells.

In certain embodiments, the isolated population cells are T cells. In some embodiment, the isolated population of T cells can be selected from the group consisting of CD4+/CD8+ double positive T cells, cytotoxic T cells, Th3 (Treg) cells, Th9 cells, Thαβ helper cells, Tfh cells, stem memory TSCM cells, central memory TCM cells, effector memory TEM cells, effector memory TEMRA cells, gamma delta T cells and any combination thereof.

In some embodiments, the isolated population of cells is derived from a cell type that is easily accessible and requires minimal invasion, such as a fibroblast, a skin cell, a cord blood cell, a peripheral blood cell, and a renal epithelial cell.

In certain embodiments, the isolated population of cells are terminally differentiated cells. In certain embodiments, the isolated population of cells are terminally differentiated T cells. In certain embodiments, the isolated population of cells are terminally differentiated PBMC cells. In certain embodiments, the isolated population of cells are terminally differentiated γδ T cells.

The isolated population of cells of the present disclosure may be derived from a mammal, preferably a human, but include and are not limited to non-human primates, murines (i.e., mice and rats), canines, felines, equines, bovines, ovines, porcines, caprines, etc.

In certain embodiments, the isolated population of cells are mammal cells.

In certain embodiments, the isolated population of cells are human cells.

In some embodiments, the isolated population of cells are human PBMC cells.

Introduction of Pluripotency-Associated Genes

The present disclosure also relates to introducing into the activated population of cells an endogenous gene locus that is a pluripotency-associated gene. In some embodiments, such pluripotency-associated gene can be introduced using an expression vector. In some embodiments, such pluripotency-associated gene can be introduced using the CRISPR activation system with at least one sgRNA targeting the desired gene locus. In some embodiments, such pluripotency-associated gene can be introduced by expression from a recombinant expression cassette that has been introduced into the target cell. In some embodiments, such pluripotency-associated gene can be introduced by incubating the cells in the presence of exogenous reprogramming transcription factor polypeptides.

In certain embodiments, the expression vector used to introduce pluripotency-associated gene includes a modified viral polynucleotide, such as from an adenovirus, a Sendai virus, a herpesvirus, or a retrovirus, such as a lentiviral vector. The expression vector is not restricted to recombinant viruses and includes non-viral vectors such as DNA plasmids and in vitro transcribed mRNA. In one preferred embodiment, Sendai virus vector is used.

To address the safety issues that arise from target cell genomes harboring integrated exogenous sequences, a number of modified genetic protocols have been developed and can be used in the methods of producing described herein. These protocols produce iPS cells with potentially reduced risks, and include non-integrating adenoviruses to deliver reprogramming genes (Stadtfeld, M., et al., Science, 2008, 322:945-949), transient transfection of reprogramming plasmids (Okita, K., et al., Science, 2008, 322:949-953), piggyBac transposition systems (Woltjen, K., et al., Nature, 2009, 458:766-770; Yusa, et al., Nat. Methods, 2009, 6:363-369; Kaji, K., et al. (2009)), Cre-excisable viruses (Soldner, F., et al., Cell, 2009, 136:964-977), and oriP/EBNA1-based episomal expression system (Yu, J., et al., Science, 2009, 324(5928): 797-801).

Non-limiting examples of a pluripotency-associated gene (gene that encode a reprogramming transcription factor) are Oct3/4, Sox2, Nanog, Klf4, c-Myc, Nanog, Lin28, Nr5a2, Glis1, Cebpa, Esrrb, and Rex1. In some embodiments, the endogenous gene locus is Oct4 or Sox2.

In certain embodiments, the isolated population of cells endogenously express at least one or more proteins from the group consisting of Oct3/4 polypeptide, a Klf4 polypeptide, a c-Myc polypeptide, a Sox2 polypeptide, a Nanog polypeptide, a Lin28 polypeptide, a Nr5a2 polypeptide, a Glis1 polypeptide, a Cebpa polypeptide, a Esrrb polypeptide, and a Rex1 polypeptide. In certain embodiments, the isolated population of cells do not endogenously express any reprogramming transcription factor.

In certain embodiment, the reprogramming factors comprise Oct3/4, Sox2, Klf4, and c-Myc.

In certain embodiments, the reprogramming factors are Oct3/4, Sox2, KLF4, c-Myc, and Lin28.

In certain embodiments, the reprogramming factors are Oct3/4, Sox2, Klf4, c-Myc.

The exogenously introduced pluripotency gene may be carried out in several ways. In one embodiment, the exogenously introduced pluripotency gene may be expressed from a chromosomal locus different from the endogenous chromosomal locus of the pluripotency gene. Such chromosomal locus may be a locus with open chromatin structure, and contain gene(s) dispensable for a somatic cell. In other words, the desirable chromosomal locus contains gene(s) whose disruption will not cause cells to die. Exemplary chromosomal loci include, for example, the mouse ROSA 26 locus and type II collagen (Col2a1) locus (See Zambrowicz et al., 1997).

The exogenously introduced pluripotency gene may be expressed from an inducible promoter such that their expression can be regulated as desired.

In an alternative embodiment, the exogenously introduced pluripotency gene may be transiently transfected into cells, either individually or as part of a cDNA expression library, prepared from pluripotent cells. Such pluripotent cells may be embryonic stem cells, oocytes, blastomeres, inner cell mass cells, embryonic germ cells, embryoid body (embryonic) cells, morula-derived cells, teratoma (teratocarcinoma) cells, and multipotent partially differentiated embryonic stem cells taken from later in the embryonic development process.

The cDNA library is prepared by conventional techniques. Briefly, mRNA is isolated from an organism of interest. An RNA-directed DNA polymerase is employed for first strand synthesis using the mRNA as template. Second strand synthesis is carried out using a DNA-directed DNA polymerase which results in the cDNA product. Following conventional processing to facilitate cloning of the cDNA, the cDNA is inserted into an expression vector such that the cDNA is operably linked to at least one regulatory sequence. The choice of expression vectors for use in connection with the cDNA library is not limited to a particular vector. Any expression vector suitable for use in mouse cells is appropriate. In one embodiment, the promoter which drives expression from the cDNA expression construct is an inducible promoter. The term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express cDNAs. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

The exogenously introduced pluripotency gene may be expressed from an inducible promoter. The term “inducible promoter”, as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotic.

A tetracycline-inducible promoter is an example of an inducible promoter that responds to an antibiotics. See Gossen et al., 2003. The tetracycline-inducible promoter comprises a minimal promoter linked operably to one or more tetracycline operator(s). The presence of tetracycline or one of its analogues leads to the binding of a transcription activator to the tetracycline operator sequences, which activates the minimal promoter and hence the transcription of the associated cDNA. Tetracycline analogue includes any compound that displays structural homologies with tetracycline and is capable of activating a tetracycline-inducible promoter. Exemplary tetracycline analogues includes, for example, doxycycline, chlorotetracycline and anhydrotetracycline.

Thus, in one embodiment, the present disclosure provides somatic cells carrying at least one pluripotency gene expressed as a transgene under an inducible promoter. It is possible that somatic cells with such inducible pluripotency transgene(s) are more prone to be reprogrammed.

Any of the engineered somatic cells of the present disclosure may be used in the methods. In one embodiment, somatic cells used in the methods comprise only one endogenous pluripotency gene linked to a first selectable marker, and the selection step is carried out to select for the expression of the first selectable marker. In an alternative embodiment, the somatic cells used in the methods comprise any number of endogenous pluripotency genes, each of which is linked to a distinct selectable marker respectively, and the selection step is carried out to select for at least a subset of the selectable markers. For example, the selection step may be carried out to select for all the selectable markers linked to the various endogenous pluripotency genes.

In an alternative embodiment, somatic cells used in the method comprise a selectable marker linked to an endogenous pluripotency gene and an additional pluripotency gene expressed as a transgene under an inducible promoter. For these cells, the method of reprogramming may comprises induce the expression of the pluripotency transgene and select for the expression of the selectable marker.

In certain embodiments, in step (d) described in the method above, the transduced γδ T cells are cultured in the presence of one or more feeder layers. In certain embodiments, in step (d) the transduced γδ T cells are cultured in the presence of a mono layer of feeder cells. In certain embodiments, the feeder layer comprises mouse embryonic fibroblasts (MEFs). In certain embodiments, in step (d) the transduced γδ T cells are cultured in the presence of a feeder monolayer. In certain embodiments, in step (d) the transduced γδ T cells are cultured in the presence of mitotically inactivated mouse embryonic fibroblasts (MEFs). In certain embodiments, in step (d) the transduced γδ T cells are cultured under feeder free condition. In certain embodiment, in step (d) the transduced γδ T cells are cultured in iMatrix-511 coated plates. In certain embodiments, following step (d) the method further comprises step (e) isolating and/or purifying the produced iPSCs.

In certain more specific embodiments, provided herein is a method of producing induced pluripotent stem cells (iPSCs) comprising: contacting an isolated population of cells with an activation culture; wherein the activation culture comprises IL-15, zoledronic acid and IL-2; culturing the isolated population of cells in the activation culture to enrich and/or activate γδ T cells in the isolated population of cells; transducing the γδ T cells with a Sendai virus (SeV) vector encoding one or more reprogramming factors; and culturing the transduced γδ T cells under conditions suitable for reprogramming mammalian somatic cells to a pluripotent state.

In certain more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture to enrich and/or activate γδ T cells in the isolated population of cells; transducing the γδ T cells with a Sendai virus (SeV) vector encoding one or more reprogramming factors; and culturing the transduced γδ T cells under conditions suitable for reprogramming mammalian somatic cells to a pluripotent state.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days to enrich and/or activate γδ T cells in the isolated population of cells; transducing the γδ T cells with a Sendai virus (SeV) vector encoding one or more reprogramming factors; and culturing the transduced γδ T cells under conditions suitable for reprogramming mammalian somatic cells to a pluripotent state.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days so that after being cultured in the activation culture the isolated population of cells comprise less than 35% γδ T cells; transducing the γδ T cells with a Sendai virus (SeV) vector encoding one or more reprogramming factors; and culturing the transduced γδ T cells under conditions suitable for reprogramming mammalian somatic cells to a pluripotent state.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days so that after being cultured in the activation culture the isolated population of cells comprise less than 35% γδ T cells; further enriching γδ T cells by cell-cell clump enrichment; transducing the γδ T cells with a Sendai virus (SeV) vector encoding one or more reprogramming factors; and culturing the transduced γδ T cells under conditions suitable for reprogramming mammalian somatic cells to a pluripotent state.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days so that after being cultured in the activation culture the isolated population of cells comprise less than 35% γδ T cells; optionally further enriching γδ T cells by cell-cell clump enrichment; transducing the γδ T cells with a Sendai virus (SeV) vector encoding one or more reprogramming factors selected from a group consisting of OCT3/4, SOX2, KLF4, LIN28, and c-Myc; and culturing the transduced γδ T cells under conditions suitable for reprogramming mammalian somatic cells to a pluripotent state.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days so that after being cultured in the activation culture the isolated population of cells comprise less than 35% γδ T cells; optionally further enriching γδ T cells by cell-cell clump enrichment; transducing the γδ T cells with a Sendai virus (SeV) vector encoding one or more reprogramming factors selected from a group consisting of OCT3/4, SOX2, KLF4, LIN28, and c-Myc; and culturing the transduced γδ T cells under conditions suitable for reprogramming mammalian somatic cells to a pluripotent state in the presence of a monolayer of feeder cells.

In other more specific embodiments, provided herein is a method of producing induced pluripotent stem cells (iPSCs) comprising: obtaining an isolated population of cells (e.g., terminally differentiated cells such as peripheral blood mononuclear cells (PBMCs)) from a subject (e.g., a human); contacting an isolated population of cells with an activation culture; wherein the activation culture comprises IL-15, zoledronic acid and IL-2; culturing the isolated population of cells in the activation culture for about 3 days so that after being cultured in the activation culture the isolated population of cells comprise less than 35% γδ T cells; optionally further enriching γδ T cells by cell-cell clump enrichment; transducing the γδ T cells with a Sendai virus (SeV) vector encoding one or more reprogramming factors selected from a group consisting of OCT3/4, SOX2, KLF4, LIN28, and c-Myc; and culturing the transduced γδ T cells under conditions suitable for reprogramming mammalian somatic cells to a pluripotent state in the presence of a mono layer of feeder cells that comprises mouse embryonic fibroblasts (MEFs).

iPSCs Derived from γδ T Cells

In certain embodiments, the produced iPSCs are derived from γδ T cells. In certain embodiments, the produced iPSCs have rearrangement genes of TRG and TRD gene loci. In certain embodiments, the produced iPSCs do not produce or express TCRA and or TCRB or fragments thereof, such that there is no surface expression of TCRA and TCRB, detectable or otherwise.

In certain embodiments, the produced iPSCs are not derived from αβ T cells.

In certain embodiments, the produced iPSCs are negative for a Sendai virus (SeV) vector.

In certain embodiments, the produced iPSCs are genomically stable with no loss of a chromosome. In one embodiment, the genomic stability of the produced iPSCs is determined by Karyotyping analysis.

In certain embodiments, the produced iPSCs can grow and maintain in feeder free medium after adoption.

In certain embodiments, the method further comprises differentiating the produced iPSCs to a desired cell type in vitro or ex vivo. In certain embodiments, the method further comprises differentiating the produced iPSCs to a desired cell type in vitro. In certain embodiments, the method further comprises differentiating the produced iPSCs to a desired cell type ex vivo.

### 5.7. T-Cell Derived Induced Pluripotent Stem Cells (iPSCs)

Also provided here are isolated populations of induced pluripotent stem cells (iPSCs) with novel characteristics. In some embodiments, the isolated population of iPSCs comprise pluripotent cells that express one or more reprogramming factors, and comprise a nucleotide sequence encoding rearrangement of TRG and TRD genes.

In certain embodiments, the isolated populations of iPSCs are produced according to the methods described herein (e.g., in Section 5.6).

In certain embodiments, the reprogramming factors are selected from a group consisting of Oct3/4, Sox2, Klf4, c-Myc, and Lin28.

In certain embodiments, the reprogramming factors comprise Oct3/4, Sox2, Klf4, and c-Myc.

In certain embodiments, the reprogramming factors are Oct3/4, Sox2, KLF4, c-Myc, and Lin28.

In certain embodiments, the reprogramming factors are Oct3/4, Sox2, Klf4, and c-Myc.

In certain embodiments, the isolated population of iPSCs are derived from γδ T cells. In certain embodiments, the isolated population of iPSCs have rearrangement genes of TRG and TRD gene loci. In certain embodiments, the isolated population of iPSCs do not produce PCR products from TCRG and TCRD gene loci.

In certain embodiments, the isolated population of iPSCs are not derived from αβ T cells. In certain embodiments, the isolated population of iPSCs do not have rearrangement genes of TRA and TRB gene loci. In certain embodiments, the isolated population of iPSCs do not produce PCR products from TCRA and TCRB gene loci.

In certain embodiments, the isolated population of iPSCs are negative for a Sendai virus (SeV) vector.

In certain embodiments, the isolated population of iPSCs are genomically stable with no loss of a chromosome. In one embodiment, the genomic stability of the isolated population of iPSCs is determined by Karyotyping analysis.

In certain embodiments, the isolated population of iPSCs can grow and maintain in feeder free medium after adoption.

In some embodiments, provided herein is an isolated population of induced pluripotent stem cells (iPSCs) comprising pluripotent cells that express one or more reprogramming factors, wherein (i) the pluripotent cells comprise a nucleotide sequence encoding rearrangement of TRG and TRD genes or have rearrangement genes of TRG and TRD gene loci, (ii) the reprogramming factors are selected from a group consisting of Oct3/4, Sox2, Klf4, c-Myc, and Lin28, (iii) the isolated population of iPSCs are negative for a Sendai virus (SeV) vector; (iv) the isolated population of iPSCs are derived from γδ T cells, but not from αβ T cells; (v) the isolated population of iPSCs do not produce PCR products from TCRA and TCRB gene loci; (vi) the isolated population of iPSCs are genomically stable with no loss of a chromosome, e.g., as determined by Karyotyping analysis; and/or (vii) the isolated population of iPSCs can grow and maintain in feeder free medium after adoption.

In a specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and wherein the reprogramming factors are selected from a group consisting of Oct3/4, Sox2, Klf4, c-Myc, and Lin28.

In another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and wherein the reprogramming factors are Oct3/4, Sox2, Klf4, c-Myc, and Lin28.

In yet another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and wherein the reprogramming factors are Oct3/4, Sox2, Klf4, and c-Myc.

In yet another specific embodiments, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and the isolated population of iPSCs are negative for a Sendai virus (SeV) vector.

In yet another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, the reprogramming factors are selected from a group consisting of Oct3/4, Sox2, Klf4, c-Myc, and Lin28, and the isolated population of iPSCs are negative for a Sendai virus (SeV) vector.

In yet another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and the isolated population of iPSCs are derived from γδ T cells, but not from αβ T cells.

In yet another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes, and the isolated population of iPSCs do not produce PCR products from TCRA and TCRB gene loci.

In yet another specific embodiment, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes or have rearrangement genes of TRG and TRD gene loci, and the isolated population of iPSCs are genomically stable with no loss of a chromosome, e.g., as determined by Karyotyping analysis.

In yet another specific embodiments, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes or have rearrangement genes of TRG and TRD gene loci, and the isolated population of iPSCs can grow and maintain in feeder free medium after adoption.

In yet another specific embodiments, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes or have rearrangement genes of TRG and TRD gene loci, the reprogramming factors are selected from a group consisting of Oct3/4, Sox2, Klf4, c-Myc, and Lin28, the isolated population of iPSCs are negative for a Sendai virus (SeV) vector; the isolated population of iPSCs are derived from γδ T cells, but not from αβ T cells; the isolated population of iPSCs do not produce PCR products from TCRA and TCRB gene loci; the isolated population of iPSCs are genomically stable with no loss of a chromosome, e.g., as determined by Karyotyping analysis; and the isolated population of iPSCs can grow and maintain in feeder free medium after adoption.

In yet another specific embodiments, provided herein is an isolated population of induced pluripotent stem cells (iPSCs) comprising pluripotent cells that express one or more reprogramming factors, wherein the pluripotent cells comprise a nucleotide sequence encoding rearrangement of TRG and TRD genes or have rearrangement genes of TRG and TRD gene loci, the reprogramming factors are selected from a group consisting of Oct3/4, Sox2, Klf4, c-Myc, and Lin28, the isolated population of iPSCs are negative for a Sendai virus (SeV) vector; the isolated population of iPSCs do not produce PCR products from TCRA and TCRB gene loci; the isolated population of iPSCs are genomically stable with no loss of a chromosome, e.g., as determined by Karyotyping analysis; and the isolated population of iPSCs can grow and maintain in feeder free medium after adoption.

In yet another specific embodiments, provided herein is an isolated population of induced pluripotent stem cells (iPSCs) comprising pluripotent cells, wherein the pluripotent cells comprise a nucleotide sequence encoding rearrangement of TRG and TRD genes or have rearrangement genes of TRG and TRD gene loci, the isolated population of iPSCs are negative for a Sendai virus (SeV) vector; the isolated population of iPSCs are derived from γδ T cells, but not from αβ T cells; the isolated population of iPSCs do not produce PCR products from TCRA and TCRB gene loci; the isolated population of iPSCs are genomically stable with no loss of a chromosome, e.g., as determined by Karyotyping analysis; and the isolated population of iPSCs can grow and maintain in feeder free medium after adoption.

In yet another specific embodiments, provided herein is an isolated population of induced pluripotent stem cells (iPSCs) comprising pluripotent cells, wherein the pluripotent cells comprise a nucleotide sequence encoding rearrangement of TRG and TRD genes or have rearrangement genes of TRG and TRD gene loci, the isolated population of iPSCs are negative for a Sendai virus (SeV) vector; the isolated population of iPSCs do not produce PCR products from TCRA and TCRB gene loci; the isolated population of iPSCs are genomically stable with no loss of a chromosome, e.g., as determined by Karyotyping analysis; and the isolated population of iPSCs can grow and maintain in feeder free medium after adoption.

In yet another specific embodiments, provided herein is an isolated population of induced pluripotent stem cells (iPSCs) comprising pluripotent cells, wherein the pluripotent cells comprise a nucleotide sequence encoding rearrangement of TRG and TRD genes or have rearrangement genes of TRG and TRD gene loci, the isolated population of iPSCs do not produce PCR products from TCRA and TCRB gene loci; the isolated population of iPSCs are genomically stable with no loss of a chromosome, e.g., as determined by Karyotyping analysis; and the isolated population of iPSCs can grow and maintain in feeder free medium after adoption.

In yet another specific embodiments, provided herein is an isolated population of induced pluripotent stem cells (iPSCs) comprising pluripotent cells, wherein the pluripotent cells comprise a nucleotide sequence encoding rearrangement of TRG and TRD genes or have rearrangement genes of TRG and TRD gene loci, the isolated population of iPSCs do not produce PCR products from TCRA and TCRB gene loci; and the isolated population of iPSCs are genomically stable with no loss of a chromosome, e.g., as determined by Karyotyping analysis.

Also provided herein are reprogrammed somatic cells, including reprogrammed pluripotent somatic cells such as iPSCs, produced by the methods of the present disclosure.

In some embodiments, the reprogramed somatic cells of the present invention are ES-like cells, and thus may be induced to differentiate to obtain desired cell types according to known methods to differentiate ES cells. For example, the iPSCs may be induced to differentiate into hematopoietic stem cells, muscle cells, cardiac muscle cells, liver cells, cartilage cells, epithelial cells, urinary tract cells, etc., by culturing such cells in differentiation medium and under conditions which provide for cell differentiation. Medium and methods which result in the differentiation of embryonic stem cells are known in the art as are suitable culturing conditions.

In some specific embodiments, the iPSCs are induced to differentiate into hematopoietic stem cells, for example, as described in Palacios et al., Proc. Natl. Acad. Sci., USA, 92: 7530-37 (1995), which teaches the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferal of cell aggregates to a substrate which provides for cell attachment.

In other specific embodiments, the iPSC are induced to differentiate according to methods as described in Pedersen, J. Reprod. Fertil. Dev., 6: 543-52 (1994), which references numerous articles disclosing methods for in vitro differentiation of embryonic stem cells to produce various differentiated cell types including hematopoietic cells, muscle, cardiac muscle, nerve cells, among others.

In other specific embodiments, the iPSC are induced to differentiate according to Bain et al., Dev. Biol., 168:342-357 (1995), which teaches in vitro differentiation of embryonic stem cells to produce neural cells which possess neuronal properties.

These references are exemplary of reported methods for obtaining differentiated cells from embryonic or stem-like cells. These references and in particular the disclosures therein relating to methods for differentiating embryonic stem cells are incorporated by reference in their entirety herein.

Thus, using known methods and culture medium, one skilled in the art may culture the subject embryonic or stem-like cells to obtain desired differentiated cell types, e.g., neural cells, muscle cells, hematopoietic cells, etc. In addition, the use of inducible Bcl-2 or Bcl-x1 might be useful for enhancing in vitro development of specific cell lineages.

The iPSCs provided herein may be used to obtain any desired differentiated cell type.

The iPSCs produced according to the disclosure may be used to produce genetically engineered or transgenic differentiated cells. Essentially, this will be effected by introducing a desired gene or genes, or removing all or part of an endogenous gene or genes of iPSCs produced according to the invention, and allowing such cells to differentiate into the desired cell type. A preferred method for achieving such modification is by homologous recombination because such technique can be used to insert, delete or modify a gene or genes at a specific site or sites in the stem-like cell genome.

The iPSCs of the present disclosure may, for example, be used as an in vitro model of differentiation, in particular for the study of genes which are involved in the regulation of early development. Differentiated cell tissues and organs using the iPSCs may be used in drug studies.

Also provided herein are compositions comprising the iPSCs of the disclosure. In some embodiments, a composition comprises a B2M knockout iPSC or a population of B2M knockout iPSCs. In some embodiments, iPSCs are generated by reprogramming γδ T cells. In some embodiments, provided herein is a composition comprising an isolated population or subpopulation functionally enhanced derivative immune cells that have been differentiated from the iPSCs produced according to the methods provided herein.

### 5.8. Methods for Identifying an Agent that Reprograms or Contributes to Reprogramming Somatic Cells

In another aspect, provided herein are methods for identifying an agent that, alone or in combination with one or more other agents, reprograms somatic cells (e.g., T cells) to a less differentiated state. The present disclosure further provides agents identified according to the methods provided herein.

In one embodiment, the methods comprise contacting somatic cells with an activation culture comprising IL-15, zoledronic acid, and/or IL-2; contacting the somatic cells with a candidate agent and then determining whether the presence of the candidate agent results in enhanced reprogramming (e.g., increased reprogramming speed and/or efficiency) relative to that which would occur if cells had not been contacted with the candidate agent.

In some embodiments, provided herein are methods for identifying an agent that, alone or in combination with one or more other agents, reprograms somatic cells (e.g., T cells) to a less differentiated state, comprising (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) contacting an isolated population of cells with a candidate agent; (d) transducing the γδ T cells with one or more viral vector(s) encoding one or more reprogramming factors; (e) culturing the transduced γδ T cells under conditions suitable for reprogramming mammalian somatic cells to a less differentiated state, and (f) determining if at least some of the somatic cells are reprogrammed to a less differentiated state. In some embodiments, the less differentiated state is a multipotent state. In some embodiments, the less differentiated state is a pluripotent state. In certain embodiments, the activation culture further comprises one or more additional agents or compounds, for example, to improve the efficiency of activation or induction. In one embodiment, the activation culture further comprises Interleukin-2 (IL-2).

In some embodiments. the IL-15, zoledronic acid, and/or IL-2 and the candidate agent are present together in the cell culture medium, while in other embodiments the IL-15, zoledronic acid, and/or IL-2 and the candidate agent are not present together (e.g., the cells are exposed to the agents sequentially). In certain embodiments, the cells are maintained in culture for 1-20 days. In certain embodiments, the cells are maintained in culture for 1-17 days. In certain embodiments, the cells are maintained in culture for 1-15 days. In certain embodiments, the cells are maintained in culture for 1-13 days. In certain embodiments, the cells are maintained in culture for 1-11 days. In certain embodiments, the cells are maintained in culture for 1-9 days. In certain embodiments, the cells are maintained in culture for 1-7 days. In certain embodiments, the cells are maintained in culture for 1-5 days. In certain embodiments, the cells are maintained in culture for 1-3 days. In certain embodiments, the cells are maintained in culture for 12-72 hours. In certain embodiments, the cells are maintained in culture for 12-60 hours. In certain embodiments, the cells are maintained in culture for 12-48 hours. In certain embodiments, the cells are maintained in culture for 12-36 hours. In certain embodiments, the cells are maintained in culture for 12-24 hours. In certain embodiments, the cells are maintained in culture for 8-16 hours. In certain embodiments, the cells are maintained in culture for 4-8 hours. In certain embodiments, the cells are maintained in culture for 2-4 hours. The cells may be maintained in culture for, e.g., at most 13 days, at most 10 days, at most 9 days, at most 8 days, at most 7 days, at most 6 days, at most 5 days, at most 4 days, at most 3 days, at most 2 days, or at most 1 day, etc., during which time they are contacted with the IL-15, zoledronic acid, and/or IL-2 and the candidate agent for all or part of the time. In some embodiments, the agent is identified as an agent that reprograms cells if there are at least 2, 5, or 10 times as many reprogrammed cells or colonies comprising predominantly reprogrammed cells after said time period than if the cells have not been contacted with the agent.

A candidate agent can be any molecule or supramolecular complex, e.g., peptide, small organic or inorganic molecule, polysaccharide, polynucleotide, etc. which is to be tested for ability to reprogram or facilitate or enhance reprogramming cells. Candidate agents may be obtained from a wide variety of sources, as will be appreciated by those in the art, including libraries of synthetic or natural compounds. In some embodiments, candidate agents are synthetic compounds. Numerous techniques are available for the random and directed synthesis of a wide variety of organic compounds and biomolecules. In some embodiments, the candidate modulators are provided as mixtures of natural compounds in the form of bacterial, fungal, plant and animal extracts, fermentation broths, conditioned media, etc., that are available or readily produced.

In some embodiments, a library of compounds is screened. A library is typically a collection of compounds that can be presented or displayed such that the compounds can be identified in a screening assay. In some embodiments, compounds in the library are housed in individual wells (e.g., of microtiter plates), vessels, tubes, etc., to facilitate convenient transfer to individual wells or vessels for contacting cells, performing cell-free assays, etc. The library may be composed of molecules having common structural features which differ in the number or type of group attached to the main structure or may be completely random. Libraries include but are not limited to, for example, phage display libraries, peptide libraries, polysome libraries, aptamer libraries, synthetic small molecule libraries, natural compound libraries, and chemical libraries. Methods for preparing libraries of molecules are well known in the art and many libraries are available from commercial or non-commercial sources. Libraries of interest include synthetic organic combinatorial libraries. Libraries, such as, synthetic small molecule libraries and chemical libraries can comprise a structurally diverse collection of chemical molecules. Small molecules include organic molecules often having multiple carbon-carbon bonds. The libraries can comprise cyclic carbon or heterocyclic structure and/or aromatic or polyaromatic structures substituted with one or more functional groups. In some embodiments, the small molecule has between 5 and 50 carbon atoms, e.g., between 7 and 30 carbons. In some embodiments, the compounds are macrocyclic. Libraries of interest also include peptide libraries, randomized oligonucleotide libraries, and the like. Libraries can be synthesized of peptoids and non-peptide synthetic moieties. Such libraries can further be synthesized which contain non-peptide synthetic moieties which are less subject to enzymatic degradation compared to their naturally-occurring counterparts. Small molecule combinatorial libraries may also be generated. A combinatorial library of small organic compounds may comprise a collection of closely related analogs that differ from each other in one or more points of diversity and are synthesized by organic techniques using multi-step processes. Combinatorial libraries can include a vast number of small organic compounds. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. In some embodiments, mixtures containing two or more compounds, extracts or other preparations obtained from natural sources (which may comprise dozens of compounds or more), and/or inorganic compounds, etc., are screened.

In one embodiment, the methods of the disclosure are used to screen “approved drugs.” An “approved drug” is any compound (which term includes biological molecules such as proteins and nucleic acids) which has been approved for use in humans by the FDA or a similar government agency in another country, for any purpose. This can be a particularly useful class of compounds to screen because it represents a set of compounds which are believed to be safe and, at least in the case of FDA approved drugs, therapeutic for at least one purpose. Thus, there is a high likelihood that these drugs will at least be safe for other purposes.

Representative examples of libraries that could be screened include DIVERSet™, available from ChemBridge Corporation, 16981 Via Tazon, San Diego, Calif. 92127. DIVERSet contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. For example, libraries based on quinic acid and shikimic acid, hydroxyproline, santonine, dianhydro-D-glucitol, hydroxypipecolinic acid, andrographolide, piperazine-2-carboxylic acid based library, cytosine, etc., are commercially available.

In some embodiments, the candidate agents are cDNAs from a cDNA expression library prepared from cells, e.g., pluripotent cells. Such cells may be embryonic stem cells, oocytes, blastomeres, teratocarcinomas, embryonic germ cells, inner cell mass cells, etc.

It will be appreciated that the candidate reprogramming agent to be tested is typically one that is not present in standard culture medium, or if present is present in lower amounts than when used in the present invention. It will also be appreciated that a useful reprogramming agent or other form of reprogramming treatment need not be capable of reprogramming all types of somatic cells and need not be capable of reprogramming all somatic cells of a given cell type. Without limitation, a candidate agent that results in a population that is enriched for reprogrammed cells by a factor of 2, 5, 10, 50, 100 or more (i.e., the fraction of reprogrammed cells in the population is 2, 5, 10, 50, or 100 times more than present in a starting population of cells treated in the same way but without being contacted with the candidate agent) is of use.

In some embodiments, the screening method provided herein is used to identify an agent or combination of agents that substitutes for Klf4 in reprogramming cells to a pluripotent state. In some embodiments, the method is used to identify an agent that substitutes for Sox2 in reprogramming cells to a pluripotent state. In some embodiments, the method is used to identify an agent that substitutes for Oct3/4 in reprogramming cells to a pluripotent state. In some embodiments, the method is used to identify an agent that substitutes for c-Myc in reprogramming cells to a pluripotent state. In some embodiments, the method is used to identify an agent that substitutes for Lin28 in reprogramming cells to a pluripotent state. In some embodiments, the methods are practiced using human cells. In some embodiments, the methods are practiced using mouse cells. In some embodiments, the methods are practiced using non-human primate cells.

In another aspect, provided herein are methods for identifying a gene that activates the expression of an endogenous pluripotency gene in somatic cells (e.g., T cells).

In some embodiments, the method comprises (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with one or more viral vector(s) encoding one or more candidate reprogramming factors; (d) culturing the transduced γδ T cells under conditions suitable for reprogramming mammalian somatic cells to a less differentiated state, and (e) determining if at least some of the somatic cells are reprogrammed to a less differentiated state. In some embodiments, the less differentiated state is a multipotent state. In some embodiments, the less differentiated state is a pluripotent state.

In some more specific embodiments, the method comprises (a) contacting an isolated population of cells with an activation culture; wherein the activation culture comprises IL-15 and zoledronic acid; (b) culturing the isolated population of cells in the activation culture to enrich and/or activate γδ T cells in the isolated population of cells; (c) transducing the γδ T cells with one or more viral vector(s) encoding one or more candidate reprogramming factors; and (d) culturing the transduced γδ T cells under conditions suitable for reprogramming mammalian somatic cells to a pluripotent state, and (e) determining if at least some of the somatic cells are reprogrammed to a pluripotent state.

In certain embodiments, the activation culture further comprises one or more additional agents or compounds, for example, to improve the efficiency of activation or induction. In one embodiment, the activation culture further comprises Interleukin-2 (IL-2).

Therefore, in some embodiments, the method provided herein comprises (a) contacting an isolated population of cells with an activation culture; wherein the activation culture comprises IL-15, zoledronic acid and IL-2; (b) culturing the isolated population of cells in the activation culture to enrich and/or activate γδ T cells in the isolated population of cells; (c) transducing the γδ T cells with one or more viral vector(s) encoding one or more candidate reprogramming factors; (d) culturing the transduced γδ T cells under conditions suitable for reprogramming mammalian somatic cells to a less differentiated state; and (e) determining if at least some of the somatic cells are reprogrammed to a less differentiated state.

In some more specific embodiments, the method provided herein comprises: (a) contacting an isolated population of cells with an activation culture; wherein the activation culture comprises IL-15, zoledronic acid and IL-2; (b) culturing the isolated population of cells in the activation culture to enrich and/or activate γδ T cells in the isolated population of cells; (c) transducing the γδ T cells with one or more viral vector(s) encoding one or more candidate reprogramming factors; (d) culturing the transduced γδ T cells under conditions suitable for reprogramming mammalian somatic cells to a pluripotent state; and (e) determining if at least some of the somatic cells are reprogrammed to a pluripotent state.

In other embodiments, the methods comprise culturing the somatic cells as provided herein, e.g., in the presence of IL-15, zoledronic acid, and/or IL-2, and then transfecting the somatic cells of the present disclosure with a cDNA library prepared from ES cells or oocytes, selecting for cells that express the first selectable marker, and assessing the expression of the first endogenous pluripotency gene in the transfected cells that express the first selectable marker. The expression of the first endogenous pluripotency gene indicates that the cDNA encodes a gene that activates the expression of an endogenous pluripotency gene in somatic cells.

The present methods are applicable for identifying a gene that activates the expression of at least two endogenous pluripotency genes in somatic cells. The somatic cells used in the methods further comprise a second endogenous pluripotency gene linked to a second selectable marker. The methods can be modified to select for transfected cells that express both selectable markers, among which the expression of the first and the second endogenous pluripotency genes are assessed. The expression of both the first and the second endogenous pluripotency genes indicates that the cDNA encodes a gene that activates the expression of at least two pluripotency genes in somatic cells.

The present methods are further applicable for identifying a gene that activates the expression of at least three endogenous pluripotency genes in somatic cells. The somatic cells used in the methods further comprise a third endogenous pluripotency gene linked to a third selectable marker. The methods are modified to select for transfected cells that express all three selectable markers, among which the expression of all three endogenous pluripotency genes are assessed. The expression of all three endogenous pluripotency genes indicates that the cDNA encodes a gene that activates the expression of at least three pluripotency genes in somatic cells.

The practice of the present invention will employ, unless otherwise indicated, conventional techniques of mouse genetics, developmental biology, cell biology, cell culture, molecular biology, transgenic biology, microbiology, recombinant DNA, and immunology, which are within the skill of the art. Such techniques are described in the literature. See, for example, Current Protocols in Cell Biology, ed. by Bonifacino, Dasso, Lippincott-Schwartz, Harford, and Yamada, John Wiley and Sons, Inc., New York, 1999; Manipulating the Mouse Embryos, A Laboratory Manual, 3rd Ed., by Hogan et al., Cold Spring Harbor Laboratory Press, Cold Spring Harbor, N.Y., 2003; Gene Targeting: A Practical Approach, IRL Press at Oxford University Press, Oxford, 1993; and Gene Targeting Protocols, Human Press, Totowa, N.J., 2000. All patents, patent applications and references cited herein are incorporated in their entirety by reference.

### 6. EMBODIMENTS

This invention provides the following non-limiting embodiments.

In one set of embodiments (embodiment set A), provided are:
A1. A population of induced pluripotent stem cells (iPSCs), wherein the iPSCs have been generated by reprogramming γδ T cells, and the population comprises iPSCs that comprise a disrupted beta-2-microglobulin (B2M) gene
A2. The population of iPSCs of embodiment A1, wherein the disrupted B2M gene comprises a deletion of at least a portion of the nucleotide sequence of SEQ ID NO: 1 or of about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the nucleotide sequence of SEQ ID NO: 1.
A3. The population of iPSCs of embodiment A1, wherein the nucleotide sequence of the B2M gene encodes an amino acid sequence that is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to any one of SEQ ID NOs: 12-16.
A4. The population of iPSCs of any one of embodiments A1 to A3, wherein about or at least about 50%, 55%, 60%, 65%, 70%, 75%, 80%, 85%, 90%, 95%, 96%, 97%, 98%, 99%, or 100% of the iPSCs do not express a detectable level of B2M.
A5. The population of iPSCs of any one of embodiments A1 to A4, wherein the disruption comprises a deletion of at least one nucleotide base pair.
A6. The population of iPSCs of any one of embodiments A1 to A4, wherein the disruption comprises an insertion of at least one nucleotide base pair.
A7. The population of iPSCs of any one of embodiments A1 to A6, wherein the disrupted B2M gene exhibits reduced B2M expression relative to an undisrupted B2M gene.
A8. The population of iPSCs of embodiment A7, wherein the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference iPSC.
A9. The population of iPSCs of any one of embodiments A1 to A8, wherein the iPSCs comprising a disrupted B2M gene exhibit reduced expression of HLA-A, HLA-B, and/or HLA-C as compared to the expression of HLA-A, HLA-B, and/or HLA-C in a reference iPSC.
A10. The population of iPSCs of embodiment A9, wherein the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference iPSC.
A11. The population of iPSCs of any one of embodiments A8 to A10, wherein the reference iPSC is a population of iPSCs in which a B2M gene is not disrupted.
A12. The population of iPSCs of any one of embodiments A1 to A11, wherein the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene.
A13. The population of iPSCs of embodiment A12, wherein the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
A14. The population of iPSCs of embodiment A13, wherein the RNA-guided endonuclease is Cas12a (Cpf1).
A15. The population of iPSCs of embodiment A13, wherein the RNA-guided endonuclease is Acidaminococcus sp. BV3L6 Cas12a.
A16. The population of iPSCs of embodiment A13 or A14, wherein the RNA-guided endonuclease is MAD7.
A17. The population of iPSCs of any one of embodiments A12 to A16, wherein the gRNA binds to at least a portion of a complement sequence of SEQ ID NO:1.
A18. The population of iPSCs of any one of embodiments A12 to A16, wherein the gRNA binds to a complement sequence of any one of SEQ ID NOs: 2-6 or 17.
A19. The population of iPSCs of any one of embodiments A12 to A18, wherein the gRNA comprises a sequence of any one of SEQ ID NOs: 7-11 or 18.
A20. The population of iPSCs of any one of embodiments A12-A19, wherein the gRNA comprises a sequence set forth in SEQ ID NO: 18.
A21. The population of iPSCs of any one of embodiments A12 to A20, wherein gRNA consists of a sequence of any one of SEQ ID NOs: 7-11 or 18.
A22. The population of iPSCs of any one of embodiments A4 to A21, wherein the iPSCs does not have a detectable RNA transcript encoding B2M.
A23. The population of iPSCs of any one of embodiments A4 to A22, wherein the iPSCs does not have a detectable level of B2M protein.
A24. The population of iPSCs of any one of embodiments A4 to A23, wherein the iPSCs do not express a detectable level of B2M protein when analysed by flow cytometry.
A25. The population of iPSCs of any one of embodiments A12 to A24, wherein gRNA consists of SEQ ID NO: 18.
A26. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is Cas12a (Cpf1); and
the gRNA binds to a complement sequence of SEQ ID NO: 2.
A27. The population of embodiment A26, wherein the RNA-guided endonuclease is an Acidaminococcus sp. BV3L6 Cas12a.
A28. The population of embodiment A26 or A27, wherein at least about 70% of the iPSCs do not express a detectable level of B2M protein, when measured by flow cytometry
A29. The population of embodiment any one of embodiments A26 to A28, wherein at least about 100% of the iPSCs do not express a detectable level of B2M protein, when measured by flow cytometry
A30. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
      b. the gRNA binds to a sequence that is complement to SEQ ID NO: 2.
A31. The population of embodiment A30, wherein the RNA-guided endonuclease is an Acidaminococcus sp. BV3L6 Cas12a.
A32. The population of embodiment A30 or A31, wherein at least about 70% of the iPSCs do not express a detectable level of B2M protein, when measured by flow cytometry.
A33. The population of any one of embodiments A30 to A32, wherein at least about 100% of the iPSCs do not express a detectable level of B2M protein, when measured by flow cytometry.
A34. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted;
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is Cas12a (Cpf1); and
      b. the gRNA binds to a sequence that is complement to SEQ ID NO: 3.
A35. The population of embodiment A34, wherein the RNA-guided endonuclease is an Acidaminococcus sp. BV3L6 Cas12a.
A36. The population of embodiment A34 or A35, wherein at least about 70% of the iPSCs do not express a detectable level of B2M protein, when measured by flow cytometry.
A37. The population of any one of embodiments A34 to A36, wherein at least about 100% of the iPSCs do not express a detectable level of B2M protein, when measured by flow cytometry.
A38. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted;
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
      b. the gRNA binds to a sequence that is complement to SEQ ID NO: 3.
A39. The population of embodiment A38, wherein the RNA-guided endonuclease is an Acidaminococcus sp. BV3L6 Cas12a.
A40. The population of embodiment A38 or A39, wherein at least about 70% of the iPSCs do not express a detectable level of B2M protein, when measured by flow cytometry.
A41. The population of any one of embodiments A38 to 40, wherein at least about 100% of the iPSCs do not express a detectable level of B2M protein, when measured by flow cytometry.
A42. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is Cas12a (Cpf1); and
      b. the gRNA binds to a sequence that is complement to SEQ ID NO: 4.
A43. The population of embodiment A38, wherein the RNA-guided endonuclease is an Acidaminococcus sp. BV3L6 Cas12a.
A44. The population of embodiment A42 or A43, wherein at least about 70% of the iPSCs do not express a detectable level of B2M protein, when measured by flow cytometry.
A45. The population of any one of embodiments A42 to A44, wherein at least about 100% of the iPSCs do not express a detectable level of B2M protein, when measured by flow cytometry.
A46. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50%-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
      b. the gRNA binds to a sequence that is complement to SEQ ID NO: 4.
A47. The population of embodiment A46, wherein the RNA-guided endonuclease is an Acidaminococcus sp. BV3L6 Cas12a.
A48. The population of embodiment A46 or A47, wherein at least about 70% of the iPSCs do not express a detectable level of B2M protein, when measured by flow cytometry.
A49. The population of any one of embodiments A46 to A48, wherein at least about 100% of the iPSCs do not express a detectable level of B2M protein, when measured by flow cytometry.
A50. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (iv) at least about 50%-100% of the iPSCs do not express a detectable level of B2M protein, when measured by flow cytometry;
   (v) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (vi) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is Cas12a (Cpf1); and
      b. the gRNA binds to a sequence that is complement to SEQ ID NO: 5.
A51. The population of induced pluripotent stem cells (iPSCs) of embodiment A46, wherein the RNA-guided endonuclease is Acidaminococcus sp. BV3L6 Cas12a (Cpf1).
A52. The population of embodiment A50 or A51, wherein at least about 70% of the iPSCs do not express a detectable level of B2M protein, when measured by flow cytometry.
A53. The population of any one of embodiments A50 to A52, wherein at least about 100% of the iPSCs do not express a detectable level of B2M protein, when measured by flow cytometry.
A54. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
      b. the gRNA binds to a sequence that is complement to SEQ ID NO: 5.
A55. The population of induced pluripotent stem cells (iPSCs) of embodiment A50, wherein the RNA-guided endonuclease is Acidaminococcus sp. BV3L6 Cas12a (Cpf1).
A56. The population of embodiment A54 or A55, wherein at least about 70% of the iPSCs do not express a detectable level of B2M protein, when measured by flow cytometry.
A57. The population of any one of embodiments A54 to A56, wherein at least about 100% of the iPSCs do not express a detectable level of B2M protein, when measured by flow cytometry.
A58. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50%400% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is Cas12a (Cpf1); and
      b. the gRNA binds to a sequence that is complement to SEQ ID NO: 6.
A59. The population of induced pluripotent stem cells (iPSCs) of embodiment A54, wherein the RNA-guided endonuclease is Acidaminococcus sp. BV3L6 Cas12a (Cpf1).
A60. The population of embodiment A58 or A59, wherein at least about 70% of the iPSCs do not express a detectable level of B2M protein, when measured by flow cytometry.
A61. The population of any one of embodiments A58 to A60, wherein at least about 100% of the iPSCs do not express a detectable level of B2M protein, when measured by flow cytometry.
A62. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
      b. the gRNA binds to a sequence that is complement to SEQ ID NO: 6.
A63. The population of induced pluripotent stem cells (iPSCs) of embodiment A58, wherein the RNA-guided endonuclease is Acidaminococcus sp. BV3L6 Cas12a (Cpf1).
A64. A population of induced pluripotent stem cells (iPSCs), wherein the iPSCs have been generated by reprogramming γδ T cells, and the population comprises iPSCs that comprise a disrupted beta-2-microglobulin (B2M) gene, wherein:
   (i) at least about 50-100% of the iPSCs do not express a detectable level of B2M protein, when measured by flow cytometry;
   (ii) the iPSCs comprising a disrupted B2M gene exhibit reduced expression of HLA-A, HLA-B, and/or HLA-C as compared to the expression of HLA-A, HLA-B, and/or HLA-C in a reference iPSC in which B2M gene is not disrupted.
   (iii) the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene, wherein:
      a. the RNA-guided endonuclease is MAD7; and
the gRNA binds to a sequence that is complement to SEQ ID NO: 17.
A65. The population of embodiment A62 or A63, wherein at least about 70% of the iPSCs do not express a detectable level of B2M protein, when measured by flow cytometry.
A66. The population of any one of embodiments A62 to A65, wherein at least about 100% of the iPSCs do not express a detectable level of B2M protein, when measured by flow cytometry.
A67. The population of any one of the preceeding embodiments, wherein the disruption comprises an insertion of at least one nucleotide base pair, wherein the insertion is an expression cassette, wherein the expression cassette encodes a therapeutic cargo, a therapeutic protein a TCR or a chimeric antigen receptor.
A68. The population of any one of the preceeding embodiments, wherein the complement sequence comprises any one of SEQ ID NOs: 19-23.

In another set of embodiments (embodiment set B), provided are:
B1. A method of producing induced pluripotent stem cells (iPSCs), wherein the method comprises:
   (a) contacting an isolated population of cells with an activation culture; wherein the activation culture comprises IL-15 and zoledronic acid;
   (b) culturing the isolated population of cells in the activation culture to enrich and/or activate γδ T cells in the isolated population of cells;
   (c) transducing the γδ T cells with a viral vector encoding one or more reprogramming factors;
   (d) culturing the transduced γδ T cells under conditions suitable for reprogramming mammalian somatic cells to a pluripotent state, thereby producing a population of iPSCs; and
   (e) contacting the population of iPSCs with an RNA-guided endonuclease or a nucleic acid encoding the RNA-guided endonuclease and a guide RNA (gRNA);
   wherein the gRNA binds to a target motif of a beta-2-microglobulin (B2M) polynucleotide sequence in the population of iPSCs; and
   wherein the contacting results in cleavage of the B2M polynucleotide sequence.
B2. The method of embodiment B1, wherein the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
B3. The method of embodiment B2, wherein the RNA-guided endonuclease is Cas12a (Cpf1).
B4. The method of embodiment B2, wherein the RNA-guided endonuclease is MAD7.
B5. The method of any one of embodiments B1 to B4, wherein the target motif comprises a portion of SEQ ID NO:1.
B6. The method of any one of embodiments B1 to B4, wherein the target motif comprises a sequence of any one of SEQ ID NOs: 2-6 or 17.
B7. The method of any one of embodiments B1 to B4, wherein the target motif consists of a sequence of any one of SEQ ID NOs: 2-6 or 17.
B8. The method of any one of embodiments B1 to B7, wherein the gRNA comprises a sequence of any one of SEQ ID NOs: 7-11 or 18.
B9. The method of any one of embodiments B1 to B7, wherein the gRNA comprises a SEQ ID NOs: 18.
B10. The method of any one of embodiments B1 to B7, wherein the gRNA consists of a sequence of any one of SEQ ID NOs: 7-11 or 18.
B12: The method of any one of embodiments B1 to B7, wherein the gRNA consists of a SEQ ID NO: 18.
B13. The method of any one of embodiments B1 to B12, wherein the cleavage of the B2M polynucleotide sequence results in reduced expression of B2M in the iPSCs as compared to the expression of B2M in a reference.
B14. The method of embodiment B13, wherein the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference.
B15. The method of embodiment B13 or B14, wherein the reduced expression of B2M is reduced by about or at least about 70% as compared to the expression of B2M in a reference.
B16. The method of any one of embodiments B1 to B15, wherein the cleavage of the B2M polynucleotide sequence results in reduced expression of HLA-A, HLA-B, and/or HLA-C as compared to the expression of HLA-A, HLA-B, and/or HLA-C in a reference.
B17. The method of embodiment B16, wherein the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference.
B18. The method of any one of embodiments B13 to B17, wherein the reference is iPSCs or a population of iPSCs without cleavage of the B2M polynucleotide sequence.
B19. The method of any one of embodiments B1 to B18, wherein the activation culture further comprises IL-2.
B20. The method of any one of embodiments B1 to B19, wherein the viral vector is a Sendai virus (SeV) vector.
B21. The method of any one of embodiments B1 to B20, wherein the method further comprises obtaining the isolated population of cells from a subject.
B22. The method of any one of embodiments B1 to B21, wherein the cells in the isolated population of cells are peripheral blood mononuclear cells (PBMCs).
B23. The method of any one of embodiments B1 to B22, wherein the cells in the isolated population of cells are terminally differentiated cells.
B24. The method of any one of embodiments B1 to B23, wherein the cells in the isolated population of cells are mammal cells.
B25. The method of any one of embodiments B1 to B24, wherein the cells in the isolated population of cells are human cells.
B26. The method of any one of embodiments B22 to B25, wherein the isolated population of cells are cultured in the activation culture for at most 13 days, at most 10 days, at most 9 days, at most 8 days, at most 7 days, at most 6 days, at most 5 days, at most 4 days, at most 3 days, at most 2 days, or at most 1 day.
B27. The method of embodiment B26, wherein the isolated population of cells is cultured in the activation culture for at most 3 days.
B28. The method of embodiment B26, wherein the isolated population of cells is cultured in the activation culture for 3 days.
B29. The method of any one of embodiments B1 to B28, wherein after being cultured in the activation culture the isolated population of cells comprises less than 90%, less than 80%, less than 70%, less than 60%, less than 50%, less than 45%, less than 40%, less than 35%, or less than 30% γδ T cells.
B30. The method of embodiment B29, wherein after being cultured in the activation culture the isolated population of cells comprises less than 35% γδ T cells.
B31. The method of any one of embodiments B1 to B30, further comprising enriching the γδ T cells in the isolated population of cells after step (b).
B32. The method of embodiment B31, wherein the γδ T cells are enriched by cell-cell clump enrichment.
B33. The method of any one of embodiments B1 to B32, wherein at least part of the γδ T cells are activated to Vγ9⁺ γδ T cells in step (b).
B34. The method of any one of embodiments B1 to B32, wherein at least part of the γδ T cells are activated to Vγ9δ2⁺ γδ T cells in step (b).
B35. The method of any one of embodiments B1 to B34, wherein the one or more reprogramming factors are selected from a group consisting of OCT3/4, SOX2, KLF4, LIN28, and c-Myc.
B36. The method of any one of embodiments B1 to B35, wherein in step (d) the transduced γδ T cells are cultured in the presence of one or more feeder layers.
B37. The method of embodiment B36, wherein in step (d) the transduced γδ T cells are cultured in the presence of a feeder monolayer.
B38. The method of embodiment B36 or B37, wherein the feeder layer comprises mouse embryonic fibroblasts (MEFs).
B39. The method of any one of embodiments B1 to B38, further comprising isolating and/or purifying the produced iPSCs.
B40. The method of any one of embodiments B1 to B39, further comprising differentiating the iPSCs ex vivo to cells of a desired cell type, thereby producing differentiated IPSCs.
B41. The method of any one of embodiments B1 to B40, wherein the produced iPSCs are negative for a Sendai virus (SeV) vector.
B42. The method of any one of embodiments B1 to B41, wherein the produced iPSCs are derived from γδ T cells.
B43. The method of any one of embodiments B1 to B41, wherein the produced iPSCs have rearrangement genes of TRG and TRD gene loci; and wherein optionally the produced iPSCs have Vγ9 and Vδ2 gene arrangements.
B44. The method of any one of embodiments B1 to B41, wherein the produced iPSCs are not derived from αβ T cells.
B45. The method of any one of embodiments B1 to B44, wherein the produced iPSCs do not produce or express TCRA and or TCRB or fragments thereof, such that there is no surface expression of TCRA and TCRB, detectable or otherwise.
B46. The method of any one of embodiments B1 to B45, wherein the produced iPSCs are genomically stable with no loss of a chromosome.
B47. The method of embodiment B46, wherein the genomic stability of the produced iPSCs is determined by Karyotyping analysis.
B48. The method of any one of embodiments B1 to B47, wherein the produced iPSCs can grow in feeder free medium after adoption.
B49. The method of any one of embodiments B1 to B48, wherein the RNA-guided endonuclease is Cas12a (Cpf1).
B50. A method of producing induced pluripotent stem cells (iPSCs), wherein the method comprises:
   (a) contacting an isolated population of human PBMCs with an activation culture; wherein the activation culture comprises IL-15, IL-2, and zoledronic acid;
   (b) culturing the isolated population of cells in the activation culture to enrich and/or activate γδ T cells in the isolated population of cells, wherein at least part of the γδ T cells are activated to Vγ9δ2⁺ γδ T cells;
   (c) transducing the γδ T cells with a Sendai virus vector encoding one or more reprogramming factors;
   (d) culturing the transduced γδ T cells under conditions suitable for reprogramming mammalian somatic cells to a pluripotent state, thereby producing a population of iPSCs; and
   (e) contacting the population of iPSCs with an RNA-guided endonuclease or a nucleic acid encoding the RNA-guided endonuclease and a guide RNA (gRNA), wherein the RNA-guided endonuclease is Cas12a or MAD7;
   wherein the gRNA binds to a target motif of a beta-2-microglobulin (B2M) polynucleotide sequence in the population of iPSCs; and
   wherein the contacting results in cleavage of the B2M polynucleotide sequence, wherein the cleavage of the B2M polynucleotide sequence results in reduced expression of B2M in the iPSCs as compared to the expression of B2M in a population of iPSCs without cleavage of the B2M polynucleotide sequence;
wherein the produced iPSCs:
   are negative for a Sendai virus (SeV) vector; and
do not produce or express TCRA and or TCRB or fragments thereof, such that there is no surface expression of TCRA and TCRB, detectable or otherwise
B51. The method of any one of the preceeding embodiments B1-B48, wherein the method further comprises selecting a B2M negative cell from the population of cells.
B52. The method of embodiment B51, wherein the selecting is performed by flow cytometric cell sorting, dilution plating for a single cell per well of a multi-well plate, magnetic bead enrichment or cartridge-based cell sorter.
B53. The method of any one of embodiments B1-B52, wherein the method further comprises introducing a vector encoding a knock-in cassette for insertion with the B2M polynucleotide sequence.
B54. The method of embodiment B53, wherein the method further comprises introducing a vector encoding a transposase, recombinase, or integrase for insertion of the knock in cassette.
B55. The method of embodiment B54, wherein the transposase, recombinase, or integrase is conjugated to a targeting moiety, wherein the moiety comprises a zinc finger, transcription activator like effector or a nuclease deficient CRISPR/CAS molecule.
B56. The method of any one of embodiments B53-B55, wherein the knock-in cassette encodes a therapeutic cargo, a therapeutic protein, a TCR or chimeric antigen receptor.
B57. An induced pluripotent stem cell (iPSC) produced according to the method of any one of embodiments B1 to B56.
B58. The population of iPSCs of embodiment B1, wherein the iPSCs are produced according to the method of any one of embodiments B1 to B57.
B58. A composition comprising the iPSC of embodiment B57.
B56. A differentiated IPSC produced according to the method of embodiment B39.
B57. A B2M-negative cell manufactured by any one of the embodiments B1-B56.
B58. A B2M-negative induced pluripotent stem cell for use in the manufacturing of a composition for the treatment of a subject in need.
B59. A B2M-negative induced pluripotent stem cell for use in the treatment of a subject in need.
B60. The B2M-negative induced pluripotent stem cell for use of embodiment B58 or Embodiment 59, wherein the cell further comprises a therapeutic cargo, a therapeutic protein, a TCR or a chimeric antigen receptor.
B61. The method of any one of embodiments B1-B56, wherein the gRNA binds to a complementary sequence.
B62. The method of B61, wherein the complementary sequence comprises any one of SEQ ID NOs: 19-24.

In another set of embodiments (embodiment set C), provided are:
C1. A method of producing induced pluripotent stem cells (iPSCs) comprising:
   (a) a step for performing a function of enriching and/or activating γδ T cells in an isolated population of cells;
   (b) a step for performing a function of reprogramming the γδ T cells to a pluripotent state, thereby producing iPSCs; and
   (c) a step for contacting the iPSCs with an RNA-guided endonuclease or a nucleic acid encoding the RNA-guided endonuclease and a guide RNA (gRNA);
   wherein the gRNA binds to a target motif of a beta-2-microglobulin (B2M) polynucleotide sequence in the iPSCs; and
   wherein the step for contacting results in cleavage of the B2M polynucleotide sequence.
C2. An induced pluripotent stem cell (iPSC) produced according to the method of embodiment C1.
C3. An isolated population of induced pluripotent stem cells (iPSCs) comprising pluripotent cells, wherein the pluripotent cells comprise a means for expressing one or more reprogramming factors, and/or wherein the pluripotent cells comprise a means for encoding rearrangement of TRG and TRD genes, and wherein the pluripotent cells comprise a means for cleaving a B2M gene.
C4. An isolated induced pluripotent stem cell (iPSC), wherein the pluripotent cell comprises a means for expressing one or more reprogramming factors, and/or wherein the cell comprises a means for encoding rearrangement of TRG and TRD genes, and wherein the cell comprises a means for cleaving a B2M gene.

### 7. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 7.1. Example 1: Generation and Characterization of β2m Knock-Out in γδ iPSC Cells

This example describes the generation of β2m knock-out in a γδ iPSC cell line using a nuclease. γδ iPSC cells were generated as disclosed in WO2021/257679 (PCT/US2021/037594), which is herein incorporated by reference in its entirety. In particular, the γδ iPSC cells utilized herein correspond to clone B as described in WO2021/257679.

iMatrix-511 Coating of 6-Well Plates

6-well plates were pre-coated with iMatrix-511 Laminin. iMatrix-511 Laminin was diluted by adding 5 μL of 0.5 mg/mL iMatrix-511 Laminin into 1 mL of DPBS. Then, between about 1 mL to about 1.5 mL of the diluted iMatrix-511 Laminin was added to each well of the 6-well plates and incubated at 37° C., 5% CO₂ for 1 hour.

7.1.1. Preparation of Reagents

The reagents used in this example are provided in Table 2.

TABLE 2
List of Reagents

| Reagent/Antibody | Catalog No | Lot No | Manufacturer |
|---|---|---|---|
| iMatrix-511 | T304 | 19L075 | Takara Bio |
| DPBS, no calcium, | 14190136 | 2070590 | Gibco |
| no magnesium | | | |
| cloneB | Not | NA | NA |
| | available | | |
| | (NA) | | |
| Stemfit Basic 04 Liquid A | NA | 20190319A | Ajinomoto, |
| | | | Japan |
| StemFit Basic 04 Liquid B | NA | 20190319A | Ajinomoto, |
| | | | Japan |
| ReLeSR | 5872 | 1000041836 | STEMCELL |
| | | | Technologies |
| Y-27632 ROCK inhibitor | SCM075 | 3498262 | Millipore |
| b2m_nuclease_gRNA1 | NA | NA | IDT |
| (SEQ ID NO: 7) | | | |
| b2m_nuclease_gRNA2 | NA | NA | IDT |
| (SEQ ID NO: 8) | | | |
| b2m_nuclease_gRNA3 | NA | NA | IDT |
| (SEQ ID NO: 9) | | | |
| b2m_nuclease_gRNA4 | NA | NA | IDT |
| (SEQ ID NO: 10) | | | |
| b2m_nuclease_gRNA5 | NA | NA | IDT |
| (SEQ ID NO: 11) | | | |
| Alt-R nuclease | 1076300 | 497844 | IDT |
| Electroporation enhancer | | | |
| Nuclease-free IDTE buffer | 11-01-02-02 | 586376 | IDT |
| Alt-R ® A.S. | 10001272 | 548593 | IDT |
| nuclease ultra | | | |
| Neon transfection system | MPK5000 | NA | Thermo |
| | | | fisher |
| Neon ™ Transfection | MPK1096K | 2K17080 | Thermo |
| System 10 μL Kit | | | fisher |
| bFGF | SRP6159- | 5B26L64480 | amsbio |
| | 10UG | | |

Preparation of crRNA

First, vials containing Alt-R crRNA were centrifuged. Each Alt-R crRNA was then dissolved to 100 μM with nuclease-free IDTE buffer (Table 3).

TABLE 3
Preparation of crRNA

| | Amount | Concentration | Volume |
|---|---|---|---|
| RNA | (nmol) | (μM) | (μL) |
| Alt-R CRISPR nuclease | 2 | 100 | 20 |
| crRNA | | | |

The vials were vortexed to provide a homogenous solution and incubated for 15 minutes at room temperature. The vials were vortexed again and spun down to collect liquid at the bottom of the vials. crRNAs were aliquoted in an amount of 5 μL each and stored at −20° C. Each crRNA was not used more than 3 freeze-thaw cycles.

Preparation of Enhancer

Alt-R nuclease electroporation enhancer was re-suspended to 100 μM in IDTE buffer in order to make a stock solution as provided in Table 4.

TABLE 4
Preparation of Enhancer

| Electroporation | Amount | Concentration | Volume |
|---|---|---|---|
| Enhancer | (nmol) | (μM) | (μL) |
| Alt-R nuclease | 2 | 100 | 20 |
| electroporation | | | |
| enhancer | | | |

7.1.2. Transfection

RNP Assembly

For each crRNA, the crRNA and nuclease was combined as provided in Table 5. The transfection mixture was incubated at room temperature for 20-30 minutes.

TABLE 5
Assembly of Ribonucleoprotein (RNP)

| Component | Amount |
|---|---|
| Alt-R ® CRISPR nuclease crRNA | 1 μL (100 pmol) |
| Alt-R ® A.S. nuclease Ultra | 1 μL (63 pmol) |
| Total volume | 2 μL |

Cell Harvesting

The medium composition was prepared as shown in Table 6.

TABLE 6
Medium Composition

| Reagent | Volume | |
|---|---|---|
| StemFit Basic 04 | 40 | mL |
| bFGF (0.3 mg/mL) | 2.66 | μL |
| Pen/Strep | 400 | μL |

The cells were harvested when the cells reached about 70-80% confluence. The spent media was aspirated from the wells and discarded. The cells were then rinsed with 1 mL of DPBS per well and 1 mL of ReLeSR was added to each well of a 6-well tissue culture treated plate. The plates were incubated in an incubator with 5% CO₂ at 37° C. for 45-60 seconds. After the incubation, ReLeSR was aspirated and discarded. The 6 well tissue culture treated plate containing the cells was further incubated with 5% CO₂ at 37° C. for 5 minutes.

About 1 mL of fresh StemFit Basic 04 complete growth medium was used to flush the cells and the detached cells were collected in a 15 mL centrifuge tube containing 3 mL of fresh complete growth media (StemFit Basic 04). The cells were suspended and centrifuged at 800 RPM for 2 minutes at room temperature. After the 2 minutes, the supernatant was discarded and the pellet was re-suspended in DPBS.

The cells were then counted using trypan blue and haemocytometer. The formula for cell counting was: total number of cells per mL=(total number of cells counted for 4 squares/4)×dilution factor×10⁴ cells/mL. In general, 50,000 cells were collected per tube, pooled together, and centrifuged at 800 RPM for 2 minutes at room temperature. The supernatant was then removed without disturbing the pellet. The cells were then resuspended by adding 10 μL of resuspension buffer T per tube (containing 50,000 cells) for one electroporation.

About 8 μL of the cell suspension was mixed with 2 μL of electroporation enhancer and 2 μL of an RNP complex. For the untransfected control, only 2 μL of enhancer was added to the cell suspension.

Neon Transfection

For Neon transfection, the electroporation settings were entered into the system as follows: 1400V, 20 msec, and 1 pulse. The neon pipette station was set up by filling the neon tube with 3 mL of electrolytic buffer (E2) and inserted into the station. A 10 μL neon tip was inserted into Neon pipette and 10 μL of the mixture was pipetted into the Neon tip avoiding air bubbles. The 10 μL neon tip containing the mixture was then inserted into the station and electroporation was initiated. After electroporation, the cells were transferred to the wells containing 2 mL of StemFit Basic 04 complete growth medium with ROCK inhibitor (10 μM final concentration). The 6-well plates were treated as shown in Table 7.

TABLE 7
6-Well Plate Map

| | | |
|---|---|---|
| Untransfected | β2m_nuclease_gRNA1 | β2m_nuclease_gRNA2 |
| | (SEQ ID NO: 7) | (SEQ ID NO: 8) |
| β2m_nuclease_gRNA3 | β2m_nuclease_gRNA4 | β2m_nuclease_gRNA5 |
| (SEQ ID NO: 9) | (SEQ ID NO: 10) | (SEQ ID NO: 11) |

The cells were then incubated at 37° C., 5% CO₂ overnight. On every day, the morphology of the cells was observed under the microscope, the spent medium was discarded, and the wells were replenished with fresh complete medium. Once the cells reached confluence, the cells were harvested using ReLeSR for further downstream applications.

7.1.3. Results

γδ iPSC cells were transfected with a) β2m_nuclease_gRNA1; b) β2m_nuclease_gRNA2; c) β2m_nuclease_gRNA3; d) β2m_nuclease_gRNA4; or e) β2m_nuclease_gRNA5 (Table 8). Table Bis directed of the gRNA sequences and target sequences. The gRNA sequences may be modified to increase the stability of short RNAs (e.g., gRNA). In some embodiments, the modifications are AltR1 and/or AltR2. The AltR1 and/or AltR2 modifications are end blocks, modifications at both ends of the gRNA sequences, or at one end of the gRNA sequences.

TABLE 8
gRNA Sequences and Target Sequences

| | Genomic Sequence (Target | |
|---|---|---|
| gRNA | Sequence) | gRNA Sequence |
| β2m_nuclease_ | ATCCATCCGACATTGAAGTTG | /rUrA rArUrU rUrCrU rArCrU rCrUrU |
| gRNA1 | (SEQ ID NO: 2) | rGrUrA rGrArU rArUrC rCrArU rCrCrG |
| | | rArCrA rUrUrG rArArG rUrUrG / (SEQ ID |
| | | NO: 7) |
| β2m_nuclease_ | TCACAGCCCAAGATAGTTAA (SEQ | /rUrA rArUrU rUrCrU rArCrU rCrUrU |
| gRNA2 | ID NO: 3) | rGrUrA rGrArU rUrCrA rCrArG rCrCrC |
| | | rArArG rArUrA rGrUrU rArA/ (SEQ ID |
| | | NO: 8) |
| β2m_nuclease_ | CTCACGTCATCCAGCAGAGAA | /rUrA rArUrU rUrCrU rArCrU rCrUrU |
| gRNA3 | (SEQ ID NO: 4) | rGrUrA rGrArU rCrUrC rArCrG rUrCrA |
| | | rUrCrC rArGrC rArGrA rGrArA / (SEQ ID |
| | | NO: 9) |
| β2m_nuclease_ | GAGTACGCTGGATAGCCTCCA | /rUrA rArUrU rUrCrU rArCrU rCrUrU |
| gRNA4 | (SEQ ID NO: 5) | rGrUrA rGrArU rGrArG rUrArC rGrCrU |
| | | rGrGrA rUrArG rCrCrU rCrCrA / (SEQ ID |
| | | NO: 10) |
| β2m_nuclease_ | AGTGGGGGTGAATTCAGTGTAGTA | /rUrA rArUrU rUrCrU rArCrU rCrUrU |
| gRNA5 | (SEQ ID NO: 6) | rGrUrA rGrArU rArGrU rGrGrG rGrGrU |
| | | rGrArA rUrUrC rArGrU rGrUrA rGrUrA / |
| | | (SEQ ID NO: 11) |

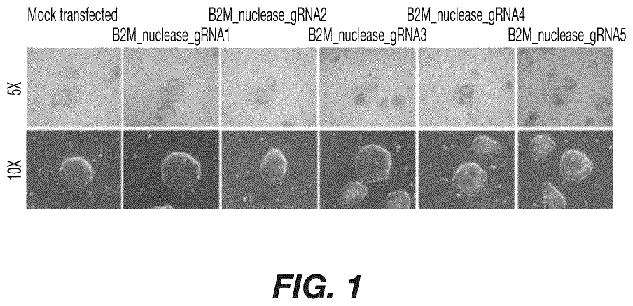

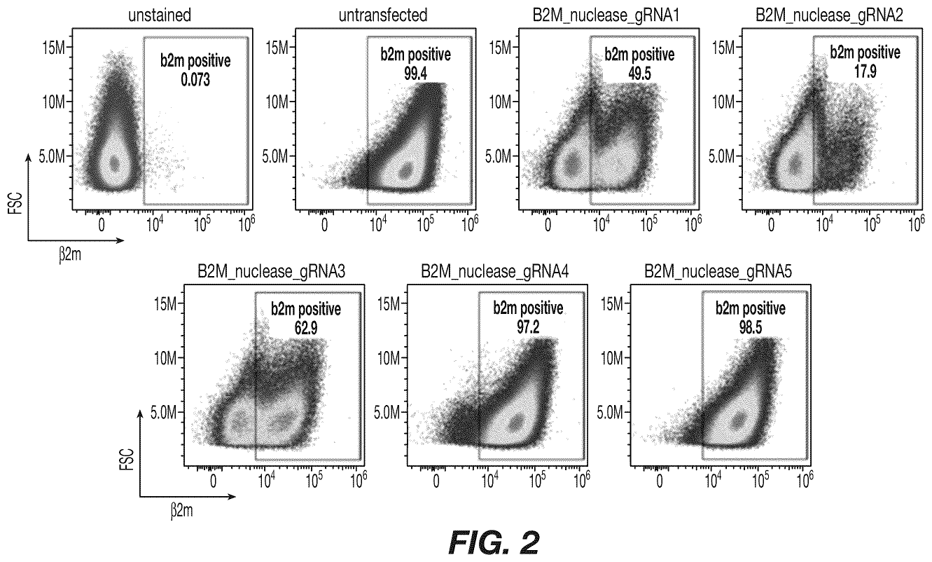

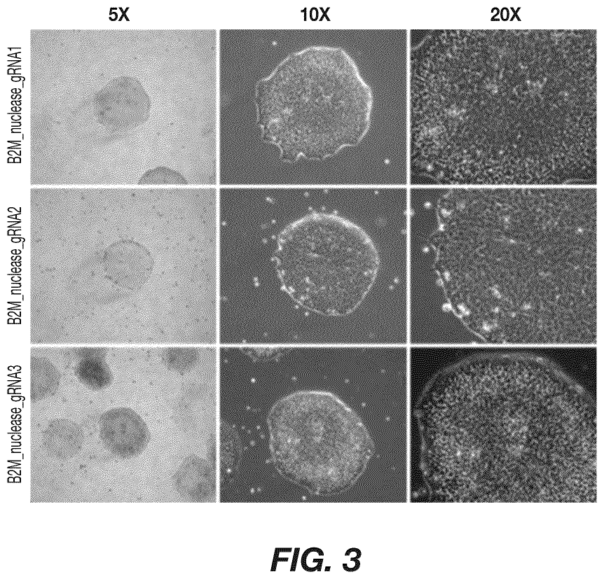

5 days post electroporation, phase contrast images of the cells were recorded as shown in FIG. 1. γδ iPSC cells were electroporated with β2m knock out crRNAs along with a nuclease and imaged 5 days post electroporation at 5× and 10× magnifications (FIG. 1). 7 days post electroporation, cells were checked for β2m expression by flow cytometry (FIG. 2). Cells were gated based on FSC-H and SSC-H (Gate E1). Gate E1 cells were further selected for live population based on pacific blue (live/dead) stain. Live cells were then gated for singlets based on FSC-H and FSC-A. Singlets were used for gating the β2m specific population based on their respective FMOs. Cells transfected with β2m_nuclease_gRNA4 and β2m_nuclease_gRNA5 did not show any significant reduction in β2m expression (FIG. 2). However, cells transfected with β2m_nuclease_gRNA1, β2m_nuclease_gRNA2 and β2m_nuclease_gRNA3 showed a significant reduction in β2m expression 7 days post electroporation (FIG. 2). FIG. 3 shows 5×, 10×, and 20×γδ iPSC images 7 days post FACS sorting. The summary of knock out (KO) efficiency observed after 7 days with each crRNA is shown in Table 9.

TABLE 9
gRNAs and Percent Knock-out

| gRNA | SEQ ID NO: | % Knock-out |
|---|---|---|
| β2m_nuclease_gRNA1 | 7 | 50.5 |
| β2m_nuclease_gRNA2 | 8 | 82.1 |
| β2m_nuclease_gRNA3 | 9 | 37.1 |
| β2m_nuclease_gRNA4 | 10 | 2.8 |
| β2m_nuclease_gRNA5 | 11 | 1.5 |

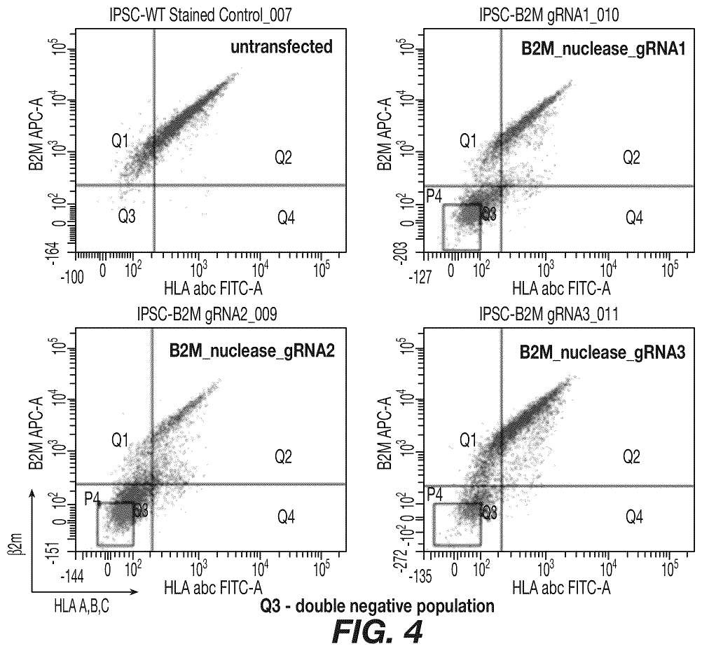

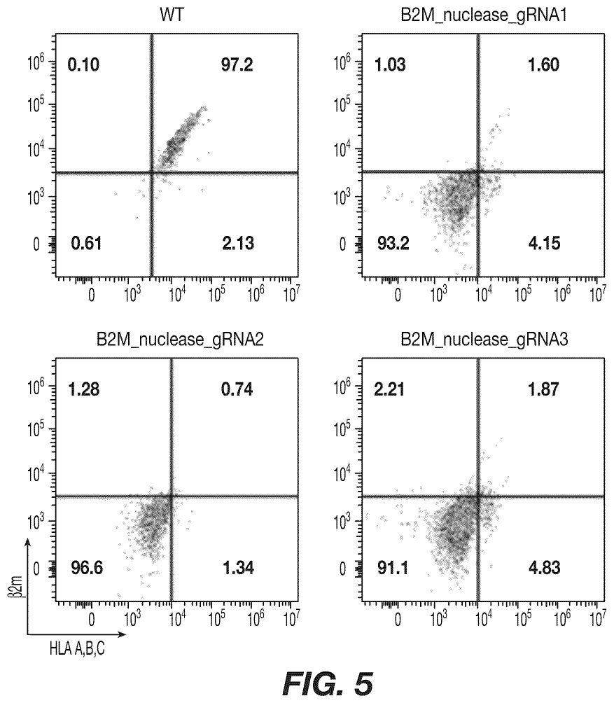

Further, cells were expanded and cell sorting of double negative population (both β2m and HLA-A, HLA-B, and HLA-C) from the total electroporated pool was performed 14 days post electroporation (FIG. 4). After FACS sorting, cells were expanded and checked again for both β2m and HLA-A, HLA-B, HLA-C expression by flow cytometry 22 days post electroporation. Cells were gated based on FSC-H and SSC-H (Gate E1). Gate E1 cells were further selected for live population based on pacific blue (live/dead) stain. Live cells were then gated for singlets based on FSC-H and FSC-A. Singlets were used for gating the β2m and HLA A, HLA-B, HLA-C specific population based on their respective FMOs. Flow cytometry analysis showed that cells electroporated with crRNA 1, 2, and 3 showed 93.2%, 96.6% and 91.1% reduction in both β2m and HLA-A, HLA-B, HLA-C protein levels, respectively (FIG. 5).

This example demonstrates the successful production of β2m gene knock-out clones that were generated from γδ iPSC cell line using CRISPR nuclease technology. The data presented herein shows that robust knock-out efficiency (up to 82.1%) was observed as early as 7 days post electroporation with β2m_nuclease_gRNA2. Three β2m KO clones were generated within 22 days post electroporation with >90% KO efficiency using CRISPR nuclease technology in the γδ iPSC cells. The time taken for generation of β2m knock-outs in the γδ iPSC cells using CRISPR nuclease technology was around 4 weeks.

TABLE 10
Sequences

| | | SEQ |
|---|---|---|
| | | ID |
| Name | Sequence | NO: |
| Beta-2- | TTTAAACTGCTGACAACTTAACACTGATTACTCATAGAGGCACTGC | 1 |
| microglobulin | CATGGTGGTCTTGGATAAGATCTGGAAGAATTCTCTGGATTACCAG | |
| (B2M) | GCAGAGACTCTTATTGTCTTCCCTTATTTTCCCCCAGGCAAAGTCT | |
| gene | GTCTTTCTGTGATCAGCTGCCTGGAGTTGGGGGAGAAGTGACACAA | |
| NCBI | ACACCCCTGTGACCACCACTGGGACTGTGCAGGGTCAGACGTGAAG | |
| Reference | CCAGCATAGTACTGGGTCTTGCCCAAGGCCTATGGTAACCACCGCC | |
| Sequence: | TGGCTACTACCTATGTTCACTCAAGGCCCTATGGCTCTAAAATCAG | |
| NG_012920.2 | CAGGTGGCAAAGCCAGCCAAGCTTATGTCCCTCTCTTTAGGGTGGC | |
| | AAGTTTCCCCCAGCCTGGGGTGAATCCAGAGATGCCTTCCAGGAAC | |
| | TGGGGCCTAGTCAGAAACCTTAGGAATCTACCTGGTACTCTATTGC | |
| | ACTGAGGCTGAACTGGCACCCAAGCCACAAGACAAAGTCCTTCCCA | |
| | CTTTTCCCTCCCCTTTCCACAAGCTGAGGAGTCTCTTGCCATGGCC | |
| | ACTTCCACCCCAGGCCTGTGGTGGGTACTGTCTGGCTACTGCTGAT | |
| | GTATACTCAAGGCCCAAGGGCTCTGCAGTAAGCTTGTGGTGAACGC | |
| | TGCCCAGCCTGGGACTCACCCTTCAGGGAAGCAGGCTCCCCTCTGG | |
| | CCCAGAGCAGGTCCAGGAATGCTATCTAAGAGCCAAAGCCTGGAAT | |
| | CAGGGACCTTAAGAGGCCACTTGGTATTCTAACCCCACAGTGGCCA | |
| | AGTCCCCTTTACTCTTTCCACTTCTTTTCTCAAGCAGAAGGAATTT | |
| | CTCCCCATAGCCACCACAGCTGTAATGTGCTGGGTCACACCTTAAG | |
| | CCTGCACGTCTCTGAGTCTCACCCAAAGCCCATGATGAGTACTACC | |
| | TGGCTACTTCTGATTATTCAGGGCCAAAGGGCTCTTTAGTCAGCTG | |
| | GTGAGGAATCCTGCCAGGACTGGTCCTTCCCTTCAAGGCAGCAGGT | |
| | TCCCTTCTGGCCCAGGGTGTGGCTAGAAATGTCAGCTTGGAGCTAT | |
| | GGCCTGGAATGTGGGCCTCAGGACTTTGCCTGGTGCCCTATCCTAC | |
| | TGCAGCTGAGCTGGTATCCAATGCAAGACAAACTCATCTTTGTTCT | |
| | TCCCTCTCCTCTTCTCAAGCAGAGGAAAGAAGTCTCTCTCTGAGCT | |
| | TTGAGCTGCCCTGCCTGGGGTTGGGAGAGGGGTGGTGCAAGCAATC | |
| | CCTTGGCCATCCCAGCTAGTGTCTCACTAGGTTGTGTGCCCCCCAC | |
| | ATTCACTGGCTCTGAGCCCAGCACAGCACCAGGACTTACCCAGGAA | |
| | TTGCCATCCTTGTGGCCTAGACTGCCTTTCAAGTTTATGTAGAACC | |
| | TCAGAGCACTTCAGCCCATGGTGGGGAGGCTTACTGGAACTCAGGT | |
| | TCCCACTGCTGGGATGGGTGATTTGCCTCTAGCTAGGGCTGGTCTA | |
| | AATGATCCCTCCATGGGACCTGGGTGAGTTCTTCCCAGTGTTGCTT | |
| | TGTGCTATGACTGGGCAGCACTGAGTTCCAATGCAAAGTCTGAAAA | |
| | TCACTGCACTCTTCCTCTCCCAGAAGCACACATTCTCTCTCCATGC | |
| | CAGGTGGCCGCTGCCGGCGAATGGGGGAGAGATAGCATTTAGCAAT | |
| | TCAAGACTGTCTTACCCTCTTCAGTTACTCTTTCAGTGATACGAAG | |
| | TTAAAACCAGGTACTGTGATCACTCACCTGATTTTTGGTTCTTATG | |
| | AAGGTGCTTTTTTTTGTGTGGATCATTATTCAATTTGGTGTTCCTG | |
| | CAGGGAGCAGGATTAGTGGAGGCGTCTATTCAGCCATCTTGCTCCA | |
| | CCTCCCCTAGCTATGTCCTTTTAAATATTTATAATCGACATACAAT | |
| | ATATTACACTTAAAGTATACAATAAGTTCTAAAATATAACTGAAAT | |
| | CATCATCGAACTCTAGATAATTAACATATCCATCAACCCTAAAGTT | |
| | TCCTCATGTTTAGATGATCTCCTTTCCCTCTAGAAACCACTCATCT | |
| | ACTTTCTGTCATATAGATTATAGTTTGCCTTTTCTGGAGTTTTATA | |
| | TAAACAGAATCATATAGTATATGTTCTTTTCTGTCTGACTTCTTTT | |
| | ATTTACCATGATTATTTCATCCATGTTACATGTATCAACAGTTTAT | |
| | TCCTTGTTTGTTGATGATAGTATTTCATTGTATGGATATATACTCC | |
| | AATGGGTTTATTCATTTACCTGTTGATGTATGTTGGATCGTTTCTA | |
| | GTTTTCAGCTATCACAAATAAAACTGCTATAAACATTAAACATTAA | |
| | TGTCCAAGTCTTTGTATAGACATATGCTTTCATTTTCTCTTGGGCA | |
| | AATACCTTGGGTGGAGTGGCTGGATATTATGGCCAGTGTATGTTTA | |
| | ACTTTATAAAGAGCTGTCGAAGTGTTTTCCTTTTTCTTTTTTCTTT | |
| | TTGAGATGAAGTCTTGCTCTGTTGCCCAGGCTGGAGTGCAGTGGTG | |
| | CAATCTCGGCTCATTGCAACCTCTGCCTCCCAGGTTCAAGCAATTC | |
| | TCCTGCCTCAGCCTCCTGAGTAGTTGGGGTTACAGATGCCTGCCAC | |
| | CATGCCTAGCTAATTTTTGTATTTTTAGTAGAGACAGGGTTTCACC | |
| | ATGTTGGCCAGGCTGGTCTCAAACTCCTGATCTCAAGTGATCTGCC | |
| | CGCCTTGACCTCCCAAAGTGCTCAGATTACAGGTGTGAGCCACCAC | |
| | ATCCAGCCCCAAATTGTTTATACCATTTTATATTCCTACCAGGATT | |
| | GTATGAGAGTTCCAGTTCTTCTAAATTCTTGCCAACATTTACTACT | |
| | TTGAATAAATGTTCATTGTAAAAAAATTTTAAGAGGGTTATATAGG | |
| | GGAAAAAAAGGCCGCTATTAATCTGTTAATTGCACTACTGGGAGAT | |
| | AAAGCTATTAAACTGTCATATTTGTGGCTGGGCGTGGTAACACATG | |
| | CTTGTGATCCTAGCACTTTAGGAGGCTGGGGAAGGAGGATCACTTG | |
| | AGACCAGGAGTTCGAGACCAGCCTGGGCAACACAGCAAGACTCTGT | |
| | CTCCACAAAAATATTAAAAATTAAAAAAAAAAGAAATTGTCATGTT | |
| | TGTAGACTTCCCAAATTTGCCATCCTAGGCAAACAGTGCTCTTGTT | |
| | TTTAAACAAATAGTTGTATACATAATTTTCCCCCTCACTATTCAAA | |
| | ACATGTCATAAATGGTAAGTCCAAGAAAAATACAGGTATTCCCCCC | |
| | CAAAGAAAACTGTAAAATCGACTTTTTTCTATCTGTACTGTTTTTT | |
| | ATTGGTTTTTAAATTGGTTTTCCAAGTGAGTAAATCAGAATCTATC | |
| | TGTAATGGATTTTAAATTTAGTGTTTCTCTGTGATGTAGTAAACAA | |
| | GAAACTAGAGGCAAAAATAGCCCTGTCCCTTGCTAAACTTCTAAGG | |
| | CACTTTTCTAGTACAACTCAACACTAACATTTCAGGCCTTTAGTGC | |
| | CTTATATGAGTTTTTAAAAGGGGGAAAAGGGAGGGAGCAAGAGTGT | |
| | CTTAACTCATACATTTAGGCATAACAATTATTCTCATATTTTAGTT | |
| | ATTGAGAGGGCTGGTAGAAAAACTAGGTAAATAATATTAATAATTA | |
| | TAGCGCTTATTAAACACTACAGAACACTTACTATGTACCAGGCATT | |
| | GTGGGAGGCTCTCTCTTGTGCATTATCTCATTTCATTAGGTCCATG | |
| | GAGAGTATTGCATTTTCTTAGTTTAGGCATGGCCTCCACAATAAAG | |
| | ATTATCAAAAGCCTAAAAATATGTAAAAGAAACCTAGAAGTTATTT | |
| | GTTGTGCTCCTTGGGGAAGCTAGGCAAATCCTTTCAACTGAAAACC | |
| | ATGGTGACTTCCAAGATCTCTGCCCCTCCCCATCGCCATGGTCCAC | |
| | TTCCTCTTCTCACTGTTCCTCTTAGAAAAGATCTGTGGACTCCACC | |
| | ACCACGAAATGGCGGCACCTTATTTATGGTCACTTTAGAGGGTAGG | |
| | TTTTCTTAATGGGTCTGCCTGTCATGTTTAACGTCCTTGGCTGGGT | |
| | CCAAGGCAGATGCAGTCCAAACTCTCACTAAAATTGCCGAGCCCTT | |
| | TGTCTTCCAGTGTCTAAAATATTAATGTCAATGGAATCAGGCCAGA | |
| | GTTTGAATTCTAGTCTCTTAGCCTTTGTTTCCCCTGTCCATAAAAT | |
| | GAATGGGGGTAATTCTTTCCTCCTACAGTTTATTTATATATTCACT | |
| | AATTCATTCATTCATCCATCCATTCGTTCATTCGGTTTACTGAGTA | |
| | CCTACTATGTGCCAGCCCCTGTTCTAGGGTGGAAACTAAGAGAATG | |
| | ATGTACCTAGAGGGCGCTGGAAGCTCTAAAGCCCTAGCAGTTACTG | |
| | CTTTTACTATTAGTGGTCGTTTTTTTCTCCCCCCCGCCCCCCGACA | |
| | AATCAACAGAACAAAGAAAATTACCTAAACAGCAAGGACATAGGGA | |
| | GGAACTTCTTGGCACAGAACTTTCCAAACACTTTTTCCTGAAGGGA | |
| | TACAAGAAGCAAGAAAGGTACTCTTTCACTAGGACCTTCTCTGAGC | |
| | TGTCCTCAGGATGCTTTTGGGACTATTTTTCTTACCCAGAGAATGG | |
| | AGAAACCCTGCAGGGAATTCCCAAGCTGTAGTTATAAACAGAAGTT | |
| | CTCCTTCTGCTAGGTAGCATTCAAAGATCTTAATCTTCTGGGTTTC | |
| | CGTTTTCTCGAATGAAAAATGCAGGTCCGAGCAGTTAACTGGCTGG | |
| | GGCACCATTAGCAAGTCACTTAGCATCTCTGGGGCCAGTCTGCAAA | |
| | GCGAGGGGGCAGCCTTAATGTGCCTCCAGCCTGAAGTCCTAGAATG | |
| | AGCGCCCGGTGTCCCAAGCTGGGGCGCGCACCCCAGATCGGAGGGC | |
| | GCCGATGTACAGACAGCAAACTCACCCAGTCTAGTGCATGCCTTCT | |
| | TAAACATCACGAGACTCTAAGAAAAGGAAACTGAAAACGGGAAAGT | |
| | CCCTCTCTCTAACCTGGCACTGCGTCGCTGGCTTGGAGACAGGTGA | |
| | CGGTCCCTGCGGGCCTTGTCCTGATTGGCTGGGCACGCGTTTAATA | |
| | TAAGTGGAGGCGTCGCGCTGGCGGGCATTCCTGAAGCTGACAGCAT | |
| | TCGGGCCGAGATGTCTCGCTCCGTGGCCTTAGCTGTGCTCGCGCTA | |
| | CTCTCTCTTTCTGGCCTGGAGGCTATCCAGCGTGAGTCTCTCCTAC | |
| | CCTCCCGCTCTGGTCCTTCCTCTCCCGCTCTGCACCCTCTGTGGCC | |
| | CTCGCTGTGCTCTCTCGCTCCGTGACTTCCCTTCTCCAAGTTCTCC | |
| | TTGGTGGCCCGCCGTGGGGCTAGTCCAGGGCTGGATCTCGGGGAAG | |
| | CGGCGGGGTGGCCTGGGAGTGGGGAAGGGGGTGCGCACCCGGGACG | |
| | CGCGCTACTTGCCCCTTTCGGCGGGGAGCAGGGGAGACCTTTGGCC | |
| | TACGGCGACGGGAGGGTCGGGACAAAGTTTAGGGCGTCGATAAGCG | |
| | TCAGAGCGCCGAGGTTGGGGGAGGGTTTCTCTTCCGCTCTTTCGCG | |
| | GGGCCTCTGGCTCCCCCAGCGCAGCTGGAGTGGGGGACGGGTAGGC | |
| | TCGTCCCAAAGGCGCGGCGCTGAGGTTTGTGAACGCGTGGAGGGGC | |
| | GCTTGGGGTCTGGGGGAGGCGTCGCCCGGGTAAGCCTGTCTGCTGC | |
| | GGCTCTGCTTCCCTTAGACTGGAGAGCTGTGGACTTCGTCTAGGCG | |
| | CCCGCTAAGTTCGCATGTCCTAGCACCTCTGGGTCTATGTGGGGCC | |
| | ACACCGTGGGGAGGAAACAGCACGCGACGTTTGTAGAATGCTTGGC | |
| | TGTGATACAAAGCGGTTTCGAATAATTAACTTATTTGTTCCCATCA | |
| | CATGTCACTTTTAAAAAATTATAAGAACTACCCGTTATTGACATCT | |
| | TTCTGTGTGCCAAGGACTTTATGTGCTTTGCGTCATTTAATTTTGA | |
| | AAACAGTTATCTTCCGCCATAGATAACTACTATGGTTATCTTCTGC | |
| | CTCTCACAGATGAAGAAACTAAGGCACCGAGATTTTAAGAAACTTA | |
| | ATTACACAGGGGATAAATGGCAGCAATCGAGATTGAAGTCAAGCCT | |
| | AACCAGGGCTTTTGCGGGAGCGCATGCCTTTTGGCTGTAATTCGTG | |
| | CATTTTTTTTTAAGAAAAACGCCTGCCTTCTGCGTGAGATTCTCCA | |
| | GAGCAAACTGGGCGGCATGGGCCCTGTGGTCTTTTCGTACAGAGGG | |
| | CTTCCTCTTTGGCTCTTTGCCTGGTTGTTTCCAAGATGTACTGTGC | |
| | CTCTTACTTTCGGTTTTGAAAACATGAGGGGGTTGGGCGTGGTAGC | |
| | TTACGCCTGTAATCCCAGCACTTAGGGAGGCCGAGGCGGGAGGATG | |
| | GCTTGAGGTCCGTAGTTGAGACCAGCCTGGCCAACATGGTGAAGCC | |
| | TGGTCTCTACAAAAAATAATAACAAAAATTAGCCGGGTGTGGTGGC | |
| | TCGTGCCTGTGGTCCCAGCTGCTCCGGTGGCTGAGGCGGGAGGATC | |
| | TCTTGAGCTTAGGCTTTTGAGCTATCATGGCGCCAGTGCACTCCAG | |
| | CGTGGGCAACAGAGCGAGACCCTGTCTCTCAAAAAAGAAAAAAAAA | |
| | AAAAAAGAAAGAGAAAAGAAAAGAAAGAAAGAAGTGAAGGTTTGTC | |
| | AGTCAGGGGAGCTGTAAAACCATTAATAAAGATAATCCAAGATGGT | |
| | TACCAAGACTGTTGAGGACGCCAGAGATCTTGAGCACTTTCTAAGT | |
| | ACCTGGCAATACACTAAGCGCGCTCACCTTTTCCTCTGGCAAAACA | |
| | TGATCGAAAGCAGAATGTTTTGATCATGAGAAAATTGCATTTAATT | |
| | TGAATACAATTTATTTACAACATAAAGGATAATGTATATATCACCA | |
| | CCATTACTGGTATTTGCTGGTTATGTTAGATGTCATTTTAAAAAAT | |
| | AACAATCTGATATTTAAAAAAAAATCTTATTTTGAAAATTTCCAAA | |
| | GTAATACATGCCATGCATAGACCATTTCTGGAAGATACCACAAGAA | |
| | ACATGTAATGATGATTGCCTCTGAAGGTCTATTTTCCTCCTCTGAC | |
| | CTGTGTGTGGGTTTTGTTTTTGTTTTACTGTGGGCATAAATTAATT | |
| | TTTCAGTTAAGTTTTGGAAGCTTAAATAACTCTCCAAAAGTCATAA | |
| | AGCCAGTAACTGGTTGAGCCCAAATTCAAACCCAGCCTGTCTGATA | |
| | CTTGTCCTCTTCTTAGAAAAGATTACAGTGATGCTCTCACAAAATC | |
| | TTGCCGCCTTCCCTCAAACAGAGAGTTCCAGGCAGGATGAATCTGT | |
| | GCTCTGATCCCTGAGGCATTTAATATGTTCTTATTATTAGAAGCTC | |
| | AGATGCAAAGAGCTCTCTTAGCTTTTAATGTTATGAAAAAAATCAG | |
| | GTCTTCATTAGATTCCCCAATCCACCTCTTGATGGGGCTAGTAGCC | |
| | TTTCCTTAATGATAGGGTGTTTCTAGAGAGATATATCTGGTCAAGG | |
| | TGGCCTGGTACTCCTCCTTCTCCCCACAGCCTCCCAGACAAGGAGG | |
| | AGTAGCTGCCTTTTAGTGATCATGTACCCTGAATATAAGTGTATTT | |
| | AAAAGAATTTTATACACATATATTTAGTGTCAATCTGTATATTTAG | |
| | TAGCACTAACACTTCTCTTCATTTTCAATGAAAAATATAGAGTTTA | |
| | TAATATTTTCTTCCCACTTCCCCATGGATGGTCTAGTCATGCCTCT | |
| | CATTTTGGAAAGTACTGTTTCTGAAACATTAGGCAATATATTCCCA | |
| | ACCTGGCTAGTTTACAGCAATCACCTGTGGATGCTAATTAAAACGC | |
| | AAATCCCACTGTCACATGCATTACTCCATTTGATCATAATGGAAAG | |
| | TATGTTCTGTCCCATTTGCCATAGTCCTCACCTATCCCTGTTGTAT | |
| | TTTATCGGGTCCAACTCAACCATTTAAGGTATTTGCCAGCTCTTGT | |
| | ATGCATTTAGGTTTTGTTTCTTTGTTTTTTAGCTCATGAAATTAGG | |
| | TACAAAGTCAGAGAGGGGTCTGGCATATAAAACCTCAGCAGAAATA | |
| | AAGAGGTTTTGTTGTTTGGTAAGAACATACCTTGGGTTGGTTGGGC | |
| | ACGGTGGCTCGTGCCTGTAATCCCAACACTTTGGGAGGCCAAGGCA | |
| | GGCTGATCACTTGAAGTTGGGAGTTCAAGACCAGCCTGGCCAACAT | |
| | GGTGAAATCCCGTCTCTACTGAAAATACAAAAATTAACCAGGCATG | |
| | GTGGTGTGTGCCTGTAGTCCCAGGAATCACTTGAACCCAGGAGGCG | |
| | GAGGTTGCAGTGAGCTGAGATCTCACCACTGCACACTGCACTCCAG | |
| | CCTGGGCAATGGAATGAGATTCCATCCCAAAAAATAAAAAAATAAA | |
| | AAAATAAAGAACATACCTTGGGTTGATCCACTTAGGAACCTCAGAT | |
| | AATAACATCTGCCACGTATAGAGCAATTGCTATGTCCCAGGCACTC | |
| | TACTAGACACTTCATACAGTTTAGAAAATCAGATGGGTGTAGATCA | |
| | AGGCAGGAGCAGGAACCAAAAAGAAAGGCATAAACATAAGAAAAAA | |
| | AATGGAAGGGGTGGAAACAGAGTACAATAACATGAGTAATTTGATG | |
| | GGGGCTATTATGAACTGAGAAATGAACTTTGAAAAGTATCTTGGGG | |
| | CCAAATCATGTAGACTCTTGAGTGATGTGTTAAGGAATGCTATGAG | |
| | TGCTGAGAGGGCATCAGAAGTCCTTGAGAGCCTCCAGAGAAAGGCT | |
| | CTTAAAAATGCAGCGCAATCTCCAGTGACAGAAGATACTGCTAGAA | |
| | ATCTGCTAGAAAAAAAACAAAAAAGGCATGTATAGAGGAATTATGA | |
| | GGGAAAGATACCAAGTCACGGTTTATTCTTCAAAATGGAGGTGGCT | |
| | TGTTGGGAAGGTGGAAGCTCATTTGGCCAGAGTGGAAATGGAATTG | |
| | GGAGAAATCGATGACCAAATGTAAACACTTGGTGCCTGATATAGCT | |
| | TGACACCAAGTTAGCCCCAAGTGAAATACCCTGGCAATATTAATGT | |
| | GTCTTTTCCCGATATTCCTCAGGTACTCCAAAGATTCAGGTTTACT | |
| | CACGTCATCCAGCAGAGAATGGAAAGTCAAATTTCCTGAATTGCTA | |
| | TGTGTCTGGGTTTCATCCATCCGACATTGAAGTTGACTTACTGAAG | |
| | AATGGAGAGAGAATTGAAAAAGTGGAGCATTCAGACTTGTCTTTCA | |
| | GCAAGGACTGGTCTTTCTATCTCTTGTACTACACTGAATTCACCCC | |
| | CACTGAAAAAGATGAGTATGCCTGCCGTGTGAACCATGTGACTTTG | |
| | TCACAGCCCAAGATAGTTAAGTGGGGTAAGTCTTACATTCTTTTGT | |
| | AAGCTGCTGAAAGTTGTGTATGAGTAGTCATATCATAAAGCTGCTT | |
| | TGATATAAAAAAGGTCTATGGCCATACTACCCTGAATGAGTCCCAT | |
| | CCCATCTGATATAAACAATCTGCATATTGGGATTGTCAGGGAATGT | |
| | TCTTAAAGATCAGATTAGTGGCACCTGCTGAGATACTGATGCACAG | |
| | CATGGTTTCTGAACCAGTAGTTTCCCTGCAGTTGAGCAGGGAGCAG | |
| | CAGCAGCACTTGCACAAATACATATACACTCTTAACACTTCTTACC | |
| | TACTGGCTTCCTCTAGCTTTTGTGGCAGCTTCAGGTATATTTAGCA | |
| | CTGAACGAACATCTCAAGAAGGTATAGGCCTTTGTTTGTAAGTCCT | |
| | GCTGTCCTAGCATCCTATAATCCTGGACTTCTCCAGTACTTTCTGG | |
| | CTGGATTGGTATCTGAGGCTAGTAGGAAGGGCTTGTTCCTGCTGGG | |
| | TAGCTCTAAACAATGTATTCATGGGTAGGAACAGCAGCCTATTCTG | |
| | CCAGCCTTATTTCTAACCATTTTAGACATTTGTTAGTACATGGTAT | |
| | TTTAAAAGTAAAACTTAATGTCTTCCTTTTTTTTCTCCACTGTCTT | |
| | TTTCATAGATCGAGACATGTAAGCAGCATCATGGAGGTAAGTTTTT | |
| | GACCTTGAGAAAATGTTTTTGTTTCACTGTCCTGAGGACTATTTAT | |
| | AGACAGCTCTAACATGATAACCCTCACTATGTGGAGAACATTGACA | |
| | GAGTAACATTTTAGCAGGGAAAGAAGAATCCTACAGGGTCATGTTC | |
| | CCTTCTCCTGTGGAGTGGCATGAAGAAGGTGTATGGCCCCAGGTAT | |
| | GGCCATATTACTGACCCTCTACAGAGAGGGCAAAGGAACTGCCAGT | |
| | ATGGTATTGCAGGATAAAGGCAGGTGGTTACCCACATTACCTGCAA | |
| | GGCTTTGATCTTTCTTCTGCCATTTCCACATTGGACATCTCTGCTG | |
| | AGGAGAGAAAATGAACCACTCTTTTCCTTTGTATAATGTTGTTTTA | |
| | TTCTTCAGACAGAAGAGAGGAGTTATACAGCTCTGCAGACATCCCA | |
| | TTCCTGTATGGGGACTGTGTTTGCCTCTTAGAGGTTCCCAGGCCAC | |
| | TAGAGGAGATAAAGGGAAACAGATTGTTATAACTTGATATAATGAT | |
| | ACTATAATAGATGTAACTACAAGGAGCTCCAGAAGCAAGAGAGAGG | |
| | GAGGAACTTGGACTTCTCTGCATCTTTAGTTGGAGTCCAAAGGCTT | |
| | TTCAATGAAATTCTACTGCCCAGGGTACATTGATGCTGAAACCCCA | |
| | TTCAAATCTCCTGTTATATTCTAGAACAGGGAATTGATTTGGGAGA | |
| | GCATCAGGAAGGTGGATGATCTGCCCAGTCACACTGTTAGTAAATT | |
| | GTAGAGCCAGGACCTGAACTCTAATATAGTCATGTGTTACTTAATG | |
| | ACGGGGACATGTTCTGAGAAATGCTTACACAAACCTAGGTGTTGTA | |
| | GCCTACTACACGCATAGGCTACATGGTATAGCCTATTGCTCCTAGA | |
| | CTACAAACCTGTACAGCCTGTTACTGTACTGAATACTGTGGGCAGT | |
| | TGTAACACAATGGTAAGTATTTGTGTATCTAAACATAGAAGTTGCA | |
| | GTAAAAATATGCTATTTTAATCTTATGAGACCACTGTCATATATAC | |
| | AGTCCATCATTGACCAAAACATCATATCAGCATTTTTTCTTCTAAG | |
| | ATTTTGGGAGCACCAAAGGGATACACTAACAGGATATACTCTTTAT | |
| | AATGGGTTTGGAGAACTGTCTGCAGCTACTTCTTTTAAAAAGGTGA | |
| | TCTACACAGTAGAAATTAGACAAGTTTGGTAATGAGATCTGCAATC | |
| | CAAATAAAATAAATTCATTGCTAACCTTTTTCTTTTCTTTTCAGGT | |
| | TTGAAGATGCCGCATTTGGATTGGATGAATTCCAAATTCTGCTTGC | |
| | TTGCTTTTTAATATTGATATGCTTATACACTTACACTTTATGCACA | |
| | AAATGTAGGGTTATAATAATGTTAACATGGACATGATCTTCTTTAT | |
| | AATTCTACTTTGAGTGCTGTCTCCATGTTTGATGTATCTGAGCAGG | |
| | TTGCTCCACAGGTAGCTCTAGGAGGGCTGGCAACTTAGAGGTGGGG | |
| | AGCAGAGAATTCTCTTATCCAACATCAACATCTTGGTCAGATTTGA | |
| | ACTCTTCAATCTCTTGCACTCAAAGCTTGTTAAGATAGTTAAGCGT | |
| | GCATAAGTTAACTTCCAATTTACATACTCTGCTTAGAATTTGGGGG | |
| | AAAATTTAGAAATATAATTGACAGGATTATTGGAAATTTGTTATAA | |
| | TGAATGAAACATTTTGTCATATAAGATTCATATTTACTTCTTATAC | |
| | ATTTGATAAAGTAAGGCATGGTTGTGGTTAATCTGGTTTATTTTTG | |
| | TTCCACAAGTTAAATAAATCATAAAACTTGATGTGTTATCTCTTAT | |
| | ATCTCACTCCCACTATTACCCCTTTATTTTCAAACAGGGAAACAGT | |
| | CTTCAAGTTCCACTTGGTAAAAAATGTGAACCCCTTGTATATAGAG | |
| | TTTGGCTCACAGTGTAAAGGGCCTCAGTGATTCACATTTTCCAGAT | |
| | TAGGAATCTGATGCTCAAAGAAGTTAAATGGCATAGTTGGGGTGAC | |
| | ACAGCTGTCTAGTGGGAGGCCAGCCTTCTATATTTTAGCCAGCGTT | |
| | CTTTCCTGCGGGCCAGGTCATGAGGAGTATGCAGACTCTAAGAGGG | |
| | AGCAAAAGTATCTGAAGGATTTAATATTTTAGCAAGGAATAGATAT | |
| | ACAATCATCCCTTGGTCTCCCTGGGGGATTGGTTTCAGGACCCCTT | |
| | CTTGGACACCAAATCTATGGATATTTAAGTCCCTTCTATAAAATGG | |
| | TATAGTATTTGCATATAACCTATCCACATCCTCCTGTATACTTTAA | |
| | ATCATTTCTAGATTACTTGTAATACCTAATACAATGTAAATGCTAT | |
| | GCAAATAGTTGTTATTGTTTAAGGAATAATGACAAGAAAAAAAAGT | |
| | CTGTACATGCTCAGTAAAGACACAACCATCCCTTTTTTTCCCCAGT | |
| | GTTTTTGATCCATGGTTTGCTGAATCCACAGATGTGGAGCCCCTGG | |
| | ATACGGAAGGCCCGCTGTACTTTGAATGACAAATAACAGATTTAAA | |
| | ATTTTCAAGGCATAGTTTTATACCTGATGGCCAGCTTTGTTTATTT | |
| | GACCAAGAATCTGAGTTAGCTAGTTCTAGGTACTGACAGGATAAAT | |
| | AAAACACAACACTGCTCCCGATCTTCTCAGTTTAGCAGAGGGACAG | |
| | ATATGCACTCAAATAATTAAAATATATCCTGATAAGAATATAGCAT | |
| | AGGTACGCGCGAAGAACTTGGCAATCGAAATTTTGTTGTTCAGGCT | |
| | GGGCGAGGTGTCTCATGTCTGTAATCCCAGCACTTTGGGAGGCCAT | |
| | GGTAGGATGATCGCTTGAGCCCAGGAGTCCGAGACCAGCCTGGGCA | |
| | ACATAACAAGACCCTGTCTCAATTCAAAGAATTGAATTAAAAAAAA | |
| | CAAAAAATAATTTTTTTAAAAAAGAAATGTTGTTGTTCAAGGAACA | |
| | ACAACAAAAATCTAGGGAGGTGTTAGAGAAGCCATTTGCCTGAGCT | |
| | GAGAGTAAGTTGCTAGTGGTTCTCTTGATTGGTAGGTGGGGCCTGG | |
| | GTTTCCAGGCATGGTAGCCAGGAAGGACAGCCACATGGCAGGTTTG | |
| | GGTAATTCCAAACAGTGGAGGAAGGGTGTCTGGGGGAAGACTTGTA | |
| | GGAACTCAGCTGAAAAAATTGGGGGATGATACTCTGAAAGAAAAAC | |
| | AAAGTTTTAAAATTTCTACTCTTACACTTAACACATAATGCTTCTG | |
| | TGACCGGATATTTAGGGGTTTTCCCCCCACACTCTGTTAGGAGAAA | |
| | AATTTTAGACAGATTAAATTTAACAGAGTTTAACTGAGCAAAAATG | |
| | ATTCTCGAACCAGGCAGCTGCCGGAGCCAGAATAGGTTCAAAATGA | |
| | CTCTGGGGGTGCCACATGGTTGGATGACATTTAGGGACAGAAAAAG | |
| | GAAAGTGATGTGCAGAAAATGGAAGTCAGGGGCAGAAGCAGCCAGA | |
| | TTGGTTGCAGTTCAGCATTTGCCTCATTTAAACAGGGTTTGAAGAG | |
| | TTGGCCACCTGTGATTGGCTGAGACTCTGTGGTATAAGAGTAAGTT | |
| | ACAGTCTGTTTACACATCCAGTTAGGTTACAGTTCACTATGCAGAG | |
| | AGAAATCTTTAGCCTGAACTTACACAGGGAGGCAGTTTTATTTATT | |
| | TATTTAATTTTTTTTTTTTGAGACAAGGTCTCACTCTGTCACCCGG | |
| | GCTGGAGTTCAGTGGTATGATCATGGCTTATTGCAGCCTCGACTTC | |
| | CTGGCCTCAAGCAATCCTTCCGCCTCAGGCTCTAGAGTAGCTGGGA | |
| | CTACAGGCACATGTCAGCATGCCTGGCTAATTTTGTTTTTTAATTT | |
| | TTAGTAGAGATGAACTCCTGGCCTTGCACAATTCTTTCGCCTCAGC | |
| | CTCCGAAAATGCTGGGATTACAGGTGTGAGCCACTGTGCCCAGCTA | |
| | AGGCAACTTTAGGCTAAACCTTTTTTTGAGACAGAGTTTCTCTCTT | |
| | GTTGTCTAGGCTGGAGTGCAGTTGCACCATCTTGGCTCACTGCAAC | |
| | CTCCACCTCCAGGGTTCAAGTGATTCTCGTTCCTCAGCCTCCCGAG | |
| | TAACTGGGATTACAGGCATGCGCCACCACGCCTGGCTAATTTTGTG | |
| | TTTTTAGTAGAGATGGTGTTTCACCATGTTGTCCAGGCTGGTCTCA | |
| | AACTCCTGGCCTCAAGTGATCCTCTGGCCTCAGCTTCCCAGAGTAC | |
| | TGAGATTACAGGCATGAGCCACTGTGCCCTGCCTAGGCTAAACTTA | |
| | ATTTAACAACACCAAACAATCTCCAGCAGACACCAACTGGGTATCC | |
| | CATAATTCAATTCGATTTTGATTGGATCTACCTGGAGATGGTGTCA | |
| | GATCCCGCTGGTTGAGGGTTCAGTCCCACAAGACTGCCCTCCACTT | |
| | CAGATGCCAATCACACATTGTAGGTTGTTACCTCTACTTCTGACTG | |
| | ACCAGCTGGAAACCAGAACTCCCATGACTGCCTCCTTGACTTTGGT | |
| | TAATTTGCTAGGACAGTTCATATTTACCAATCTATTATAAAAGATT | |
| | AAAGGCTACAGACGAATAACTAGATGAAAAGATGAATAGGGCTATA | |
| | TGT | |
| B2M | ATCCATCCGACATTGAAGTTG | 2 |
| Target | | |
| Motif 1 | | |
| Complement | CAACTTCAATGTCGGATGGAT | 19 |
| for B2M | | |
| Target | | |
| Motif 1 | | |
| B2M | TCACAGCCCAAGATAGTTAA | 3 |
| Target | | |
| Motif 2 | | |
| Complement | TTAACTATCTTGGGCTGTGA | 20 |
| for B2M | | |
| Target | | |
| Motif 2 | | |
| B2M | CTCACGTCATCCAGCAGAGAA | 4 |
| Target | | |
| Motif 3 | | |
| Complement | TTCTCTGCTGGATGACGTGAG | 21 |
| for B2M | | |
| Target | | |
| Motif 3 | | |
| B2M | GAGTACGCTGGATAGCCTCCA | 5 |
| Target | | |
| Motif 4 | | |
| Complement | TGGAGGCTATCCAGCGTACTC | 22 |
| for B2M | | |
| Target | | |
| Motif 4 | | |
| B2M | AGTGGGGGTGAATTCAGTGTAGTA | 6 |
| Target | | |
| Motif 5 | | |
| Complement | TACTACACTGAATTCACCCCCACT | 23 |
| for B2M | | |
| Target | | |
| Motif 5 | | |
| ß2m_ | / rUrA rArUrU rUrCrU rArCrU rCrUrU rGrUrA | 7 |
| nuclease_ | rGrArU rArUrC rCrArU rCrCrG rArCrA rUrUrG | |
| gRNA1 | rArArG rUrUrG / | |
| ß2m_ | / rUrA rArUrU rUrCrU rArCrU rCrUrU rGrUrA | 8 |
| nuclease_ | rGrArU rUrCrA rCrArG rCrCrC rArArG rArUrA | |
| gRNA2 | rGrUrU rArA/ | |
| ß2m_ | / rUrA rArUrU rUrCrU rArCrU rCrUrU rGrUrA | 9 |
| nuclease_ | rGrArU rCrUrC rArCrG rUrCrA rUrCrC rArGrC | |
| gRNA3 | rArGrA rGrArA / | |
| ß2m_ | / rUrA rArUrU rUrCrU rArCrU rCrUrU rGrUrA | 10 |
| nuclease_ | rGrArU rGrArG rUrArC rGrCrU rGrGrA rUrArG | |
| gRNA4 | rCrCrU rCrCrA | |
| ß2m_ | / rUrA rArUrU rUrCrU rArCrU rCrUrU rGrUrA | 11 |
| nuclease_ | rGrArU rArGrU rGrGrG rGrGrU rGrArA rUrUrC | |
| gRNA5 | rArGrU rGrUrA rGrUrA / | |
| B2M Target | AGTGGGGGTGAATTCAGTGTA | 17 |
| Motif 6 | | |
| Complement | TACACTGAATTCACCCCCACT | 24 |
| for B2M | | |
| Target | | |
| Motif 6 | | |
| ß2m | / rUrA rArUrU rUrCrU rArCrU rCrUrU rGrUrA | 18 |
| nuclease | rGrArU rArGrU rGrGrG rGrGrU rGrArA rUrUrC | |
| gRNA6 | rArGrU rGrUrA / | |
| B2M | MSRSVALAVLALLSLSGLEAIQRTPKIQVYSRHPAENGKSNFLNCY | 12 |
| (UnitProt | VSGFHPSDIEVDLLKNGERIEKVEHSDLSFSKDWSFYLLYYTEFTP | |
| KB | TEKDEYACRVNHVTLSQPKIVKWDRDM | |
| P61769) | | |
| B2M | MSRSVALAVLALLSLSGLEAIQRTPKIQVYSRHPADIEVDLLKNGE | 13 |
| (UnitProt | RIEKVEHSDLSFSKDWSFYLLYYTEFTPTEKDEYACRVNHVTLSQP | |
| KB | KIVKWDRDM | |
| F5H6I0) | | |
| B2M | GFHPSDIEVDLLKNGERIEKVEHSDLSFSKDWSFYLLYYTEFTPTE | 14 |
| (UnitProt | KDEYACRVNHVTLSQPKIVKWDRDM | |
| KB | | |
| H0YLF3) | | |
| B2M | MSRSVALAVLALLSLSGLERELKKWSIQTCLSARTGLSISCTTLNS | 15 |
| (UnitProt | PPLKKMSMPAV | |
| KB | | |
| J3KNU0) | | |
| B2M | MSRSVALAVLALLSLSGLEAIQHSGLLTSSSREWKVKFPELLCVWV | 16 |
| (UnitProt | SSIRH | |
| KB | | |
| Q16446) | | |

### 7.2 Example 2 Transgene Insertion in Human γδT Cell-Derived iPSCs

### A. Preparation of RNP Complex

β2m_nuclease_gRNA6Alt-R crRNA (SEQ ID NO: 18) was synthesized (IDT) and dissolved in nuclease free duplex buffer (IDT #11-05-01-12) at 200 04. RNP complex was prepared fresh on the day of nucleofection by combining 1 μg Alt-R® A.s. Cas12a (Cpf1) V3 (IDT #1081068), 200 pmol (1 μl of 200 μM) crRNA in P3 Buffer (Lonza #V4SP-3096) and 110 μg of PGA (Sigma #P4761) and incubated at room temperature for 30 minutes. After the incubation, 3 μM electroporation enhancer (IDT #1076301) was added for a total volume of 10 μL.

B. Generation of Edited human iPSCs

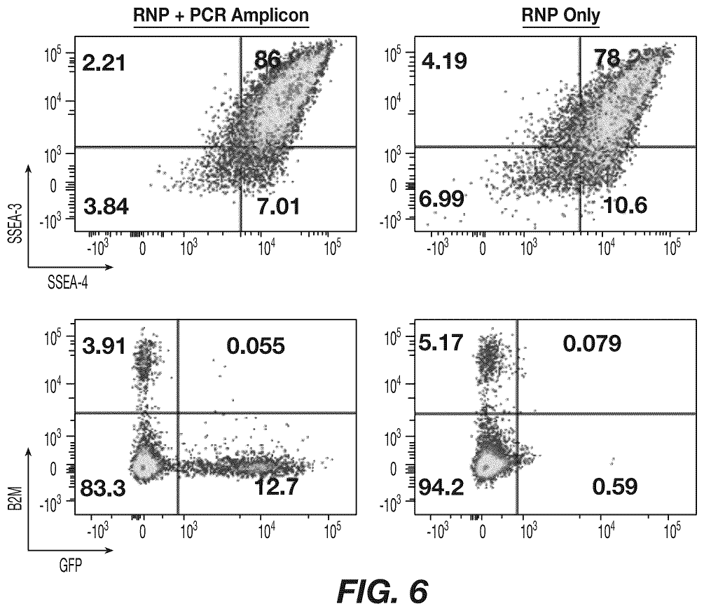

Human γδT Cell-derived iPS cells, herein referred to as “iPSCs”, were pretreated with 10 μM Y-27632 ROCK inhibitor (STEMCELL Technologies #72302) in Stemfit Basic 04 Complete Type Medium (Ajinomoto #Basic04CT). The iPSCs were collected (0.5e6 cells per reaction) and resuspended in 104, of P3 buffer with 3 μM electroporation enhancer. 2 μg of HDR template (PCR amplicon with GFP) was added to the cells and incubated for 1 minute at room temperature. The cells were combined with 10 μL of RNP complex and nucleofected in 96-well cuvettes (Lonza #V4SP-3096) using the CA-137 program on the Lonza 4D system. The iPSCs were then transferred to one well of 24-well plate coated with 0.5 μg/cm2 of iMatrix-511 (Takara #T304) containing 0.5 mL of Stemfit Basic 04 Medium with 10 μM Y-27632 ROCK inhibitor. The iPSCs were expanded to 6-well plates two days post nucleofection and the media was changed daily until day 5, at which pluripotency markers, surface expression of HLA class I, and expression of GFP were measured by flow cytometry. As shown in FIG. 6, after electroporation with a B2M targeting RNP combined with a DNA plasmid repair template encoding GFP, 12.7% of human γδT cell-derived iPSCs cells are positive for GFP and negative for B2M protein.

Accordingly, targeted insertion of the transgene into the B2M in human γδT cell-derived iPSCs was achieved.

### 7. 3 Example 3 γδT Cell-Derived iPSCs Production

A. Preparation of RNP Complex

β2m_nuclease_gRNA6 Alt-R crRNA (SEQ ID NO: 18) was synthesized (IDT) and dissolved in nuclease free duplex buffer (IDT #11-05-01-12) at 200 μM. RNP complexes were prepared fresh on the day of nucleofection by combining 1 μg Alt-R® A.s. Cas12a (Cpf1) V3 (IDT #1081068) or MAD7 (Aldevron Eureca-V MAD7), 200 pmol (1 μl of 200 μM) crRNA in P3 Buffer (Lonza #V4SP-3096) and 110 μg of PGA (Sigma #P4761) and incubated at room temperature for 30 minutes. After the incubation, 3 μM electroporation enhancer (IDT #1076301) was added for a total volume of 10 μL.

B. Generation of Edited Human iPSCs

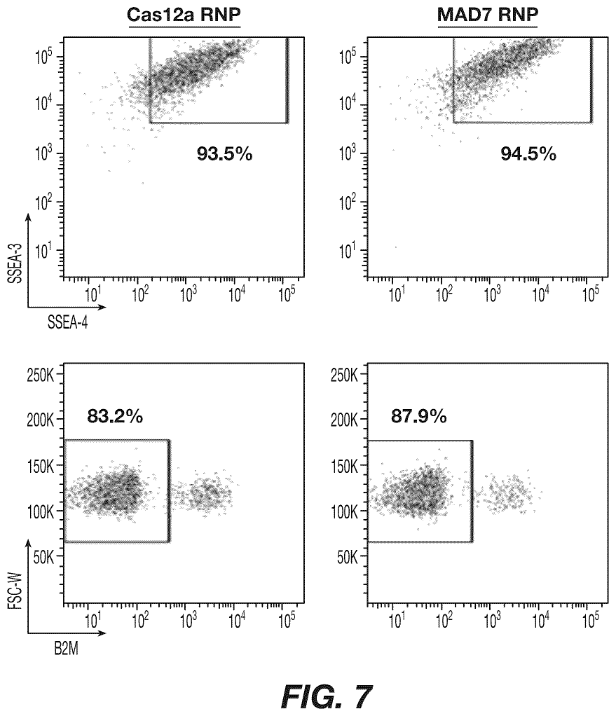

Human γδT Cell-derived iPS cells, herein referred to as “iPSCs”, were pretreated with Y-27632 ROCK inhibitor (STEMCELL Technologies #72302) in Stemfit Basic 04 Complete Type Medium (Ajinomoto #Basic04CT). The iPSCs were collected (0.5e6 cells per reaction) and resuspended in 10 μL of P3 buffer with 3 μM electroporation enhancer. The cells were combined with 10 μL of RNP complex and nucleofected in 96-well cuvettes (Lonza #V4SP-3096) using the CA-137 program on the Lonza 4D system. The iPSCs were then transferred to one well of 24-well plate coated with 0.5 μg/cm² of iMatrix-511 (Takara #T304) containing 0.5 mL of Stemfit Basic 04 Medium with 10 μM Y-27632 ROCK inhibitor. The iPSCs were expanded to 6-well plates two days post nucleofection and the media was changed daily until day 7 at which pluripotency markers and surface expression of HLA class I were measured by flow cytometry. As shown in FIG. 7, after electroporation with a B2M targeting RNP generated with Cas12a or MAD7, 83.2% and 87.9% of human γδT cell-derived iPSCs cells are negative for B2M protein, respectively.

Accordingly, electroporation with a B2M targeting RNP achieved human γδT cell-derived iPSCs cells negative for B2M protein.

### 7.4 Example 4. Human γδT Cell-Derived iPSCs with CAR

A. Preparation of RNP Complex

β2m_nuclease_gRNA6 Alt-R crRNA (SEQ ID NO: 18) (Table 10) was synthesized (IDT) and dissolved in nuclease free duplex buffer (IDT #11-05-01-12) at 200 μM. RNP complex was prepared fresh on the day of nucleofection by combining 1 μg Alt-R® A. s. Cas12a (Cpf1) V3 (IDT #1081068) with 200 pmol (1 μl of 200 μM) crRNA in P3 Buffer (Lonza #V4SP-3096) and 110 μg of PGA (Sigma #P4761) and incubated at room temperature for 30 minutes. After the incubation, 3 μM electroporation enhancer (IDT #1076301) was added for a total volume of 10 μL.

B. Generation of Edited Human iPSCs

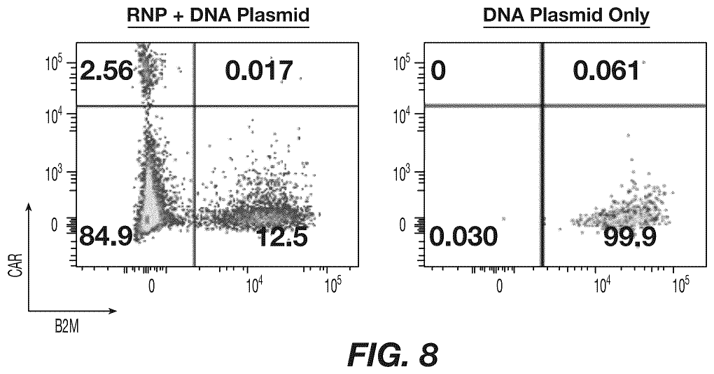

Human γδT Cell-derived iPS cells, herein referred to as “iPSCs”, were pretreated with 10 μM Y-27632 ROCK inhibitor (STEMCELL Technologies #72302) in Stemfit Basic 04 Complete Type Medium (Ajinomoto #Basic04CT). The iPSCs were collected (0.5e6 cells per reaction) and resuspended in 10 μL of P3 buffer with 3 μM electroporation enhancer. 3.5 μg of HDR template (DNA plasmid with GFP) was added to the cells and incubated for 1 minute at room temperature. The cells were combined with 10 μL of Cas12a:crRNA RNP complex and nucleofected in 96-well cuvettes (Lonza #V4SP-3096) using the CA-137 program on the Lonza 4D system. The iPSCs were then transferred to one well of 24-well plate coated with 0.5 μg/cm² of iMatrix-511 (Takara #T304) containing 0.5 mL of Stemfit Basic 04 Medium with 10 μM Y-27632 ROCK inhibitor. The iPSCs were expanded to 6-well plates two days post nucleofection and the media was changed daily until day 5, at which pluripotency markers, surface expression of HLA class I, and expression of BCMA CAR were measured by flow cytometry. As shown in FIG. 8, after electroporation with a B2M targeting RNP combined with a DNA plasmid repair template encoding a CAR, 2.6% of human γδT cell-derived iPSCs cells are positive for CAR transgene and negative for B2M protein.

Accordingly, treating with a B2M targeting RNP combined with a DNA plasmid repair template encoding a CAR achieved human γδT cell-derived iPSCs cells are positive for CAR transgene and negative for B2M protein.

What is claimed is:

1. A population of induced pluripotent stem cells (iPSCs), wherein the iPSCs have been generated by reprogramming γδ T cells, and the population comprises iPSCs that comprise a disrupted beta-2-microglobulin (B2M) gene.

2. The population of iPSCs of claim 3, wherein the disrupted B2M gene comprises a deletion of at least a portion of the nucleotide sequence of SEQ ID NO: 1 or of about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the nucleotide sequence of SEQ ID NO: 1.

3. The population of iPSCs of claim 3, wherein the nucleotide sequence of the B2M gene encodes an amino acid sequence that is about, at least about, or at most about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% identical to any one of SEQ ID NOs: 12-16.

4. The population of iPSCs of any one of claims 3 to 3, wherein about or at least about 50%, 55%, 60%, 65%, 70%, 75%, 80%, 85%, 90%, 95%, 96%, 97%, 98%, 99%, or 100% of the iPSCs do not express a detectable level of B2M.

5. The population of iPSCs of any one of claims 3 to 5, wherein the disruption comprises a deletion of at least one nucleotide base pair.

6. The population of iPSCs of any one of claims 3 to 5, wherein the disruption comprises an insertion of at least one nucleotide base pair.

7. The population of iPSCs of any one of claims 3 to 6, wherein the disrupted B2M gene exhibits reduced B2M expression relative to an undisrupted B2M gene.

8. The population of iPSCs of claim 7, wherein the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference iPSC.

9. The population of iPSCs of any one of claims 3 to 8, wherein the iPSCs comprising a disrupted B2M gene exhibit reduced expression of HLA-A, HLA-B, and/or HLA-C as compared to the expression of HLA-A, HLA-B, and/or HLA-C in a reference iPSC.

10. The population of iPSCs of claim 9, wherein the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference iPSC.

11. The population of iPSCs of any one of claims 8 to 10, wherein the reference iPSC is a population of iPSCs in which B2M gene is not disrupted.

12. The population of iPSCs of any one of claims 1 to 11, wherein the disrupted B2M gene is generated by contacting the population of iPSCs with an RNA-guided endonuclease or a nucleic acid encoding the RNA-guided endonuclease and a guide RNA (gRNA), and wherein the gRNA binds to a target motif of a B2M gene.

13. The population of iPSCs of claim 12, wherein the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

14. The population of iPSCs of claim 13, wherein the RNA-guided endonuclease is Cas12a (Cpf1).

15. The population of iPSCs of claim 13 or 14, the RNA-guided endonuclease is Acidaminococcus sp. BV3L6 Cas12a (Cpf1).

16. The population of iPSCs of claim 13, wherein the RNA-guided endonuclease is MAD7.

17. The population of iPSCs of any one of claims 12 to 16, wherein the gRNA binds to at least a portion of a complement sequence of SEQ ID NO:1.

18. The population of iPSCs of any one of claims 12 to 16, wherein the gRNA binds to a complement sequence of any one of SEQ ID NOs: 2-6 or 17.

19. The population of iPSCs of any one of claims 12 to 18, wherein the gRNA comprises a sequence of any one of SEQ ID NOs: 7-11 or 18.

20. The population of iPSCs of any one of claims 12 to 18, wherein the gRNA consists of a sequence of any one of SEQ ID NOs: 7-11 or 18.

21. The population of iPSCs of any one of claims 12 to 18, wherein the gRNA comprises SEQ ID NO: 18.

22. The population of iPSCs of any one of claims 12 to 18, wherein the gRNA consists of SEQ ID NO: 18.

23. A method of producing induced pluripotent stem cells (iPSCs), wherein the method comprises:
(a) contacting an isolated population of cells with an activation culture; wherein the activation culture comprises IL-15 and zoledronic acid;
(b) culturing the isolated population of cells in the activation culture to enrich and/or activate γδ T cells in the isolated population of cells;
(c) transducing the γδ T cells with a viral vector encoding one or more reprogramming factors;
(d) culturing the transduced γδ T cells under conditions suitable for reprogramming mammalian somatic cells to a pluripotent state, thereby producing a population of iPSCs; and
(e) contacting the population of iPSCs with an RNA-guided endonuclease or a nucleic acid encoding the RNA-guided endonuclease and a guide RNA (gRNA);
wherein the gRNA binds to a complement sequence in a target motif of a beta-2-microglobulin (B2M) polynucleotide sequence in the population of iPSCs; and
wherein the contacting results in cleavage of the B2M polynucleotide sequence.

24. The method of claim 23, wherein the RNA-guided endonuclease is selected from the group consisting of MAD7, MAD2, C2c1, C2c3, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

25. The method of claim 24, wherein the RNA-guided endonuclease is Cas12a (Cpf1).

26. The method of claim 25, wherein the RNA-guided endonuclease is Acidaminococcus sp. BV3L6 Cas12a (Cpf1).

27. The method of claim 24, wherein the RNA-guided endonuclease is MAD7.

28. The method of any one of claims 23 to 27, wherein the target motif comprises a portion of SEQ ID NO:1.

29. The method of any one of claims 23 to 27, wherein the target motif comprises a sequence of any one of SEQ ID NOs: 2-6 or 17.

30. The method of any one of claims 23 to 27, wherein the target motif consists of a sequence of any one of SEQ ID NOs: 2-6 or 17.

31. The method of any one of claims 23 to 27, wherein the gRNA comprises a sequence of any one of SEQ ID NOs: 7-11 or 18.

32. The method of any one of claims 23 to 27, wherein the gRNA comprises SEQ ID NO: 18.

33. The method of any one of claims 23 to 27, wherein the gRNA consists of a sequence of any one of SEQ ID NOs: 7-11 or 18.

34. The method of any one of claims 23 to 27, wherein the gRNA consists of SEQ ID NO: 18.

35. The method of any one of claims 23 to 34, wherein the cleavage of the B2M polynucleotide sequence results in reduced expression of B2M in the iPSCs as compared to the expression of B2M in a reference.

36. The method of claim 35, wherein the reduced expression of B2M is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of B2M in a reference.

37. The method of any one of claims 23 to 36, wherein the cleavage of the B2M polynucleotide sequence results in reduced expression of HLA-A, HLA-B, and/or HLA-C as compared to the expression of HLA-A, HLA-B, and/or HLA-C in a reference.

38. The method of claim 37, wherein the reduced expression of HLA-A, HLA-B, and/or HLA-C is reduced by about or at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% as compared to the expression of HLA-A, HLA-B, and/or HLA-C in a reference.

39. The method of any one of claims 35 to 38, wherein the reference is iPSCs or a population of iPSCs without cleavage of the B2M polynucleotide sequence.

40. The method of any one of claims 23 to 39, wherein the activation culture further comprises IL-2.

41. The method of any one of claims 23 to 40, wherein the viral vector is a Sendai virus (SeV) vector.

42. The method of any one of claims 23 to 41, wherein the method further comprises obtaining the isolated population of cells from a subject.

43. The method of any one of claims 23 to 42, wherein the cells in the isolated population of cells are peripheral blood mononuclear cells (PBMCs).

44. The method of any one of claims 23 to 43, wherein the cells in the isolated population of cells are terminally differentiated cells.

45. The method of any one of claims 23 to 44, wherein the cells in the isolated population of cells are mammal cells.

46. The method of any one of claims 23 to 45, wherein the cells in the isolated population of cells are human cells.

47. The method of any one of claims 23 to 46, wherein the isolated population of cells are cultured in the activation culture for at most 13 days, at most 10 days, at most 9 days, at most 8 days, at most 7 days, at most 6 days, at most 5 days, at most 4 days, at most 3 days, at most 2 days, or at most 1 day.

48. The method of claim 47, wherein the isolated population of cells is cultured in the activation culture for at most 3 days.

49. The method of claim 48, wherein the isolated population of cells is cultured in the activation culture for 3 days.

50. The method of any one of claims 23 to 49, wherein after being cultured in the activation culture the isolated population of cells comprises less than 90%, less than 80%, less than 70%, less than 60%, less than 50%, less than 45%, less than 40%, less than 35%, or less than 30% γδ T cells.

51. The method of claim 50, wherein after being cultured in the activation culture the isolated population of cells comprises less than 35% γδ T cells.

52. The method of any one of claims 23 to 51, further comprising enriching the γδ T cells in the isolated population of cells after step (b).

53. The method of claim 52, wherein the γδ T cells are enriched by cell-cell clump enrichment.

54. The method of any one of claims 23 to 53, wherein at least part of the γδ T cells are activated to Vγ9⁺ γδ T cells in step (b).

55. The method of any one of claims 23 to 54, wherein at least part of the γδ T cells are activated to Vγ9δ2⁺ γδ T cells in step (b).

56. The method of any one of claims 23 to 55, wherein the one or more reprogramming factors are selected from a group consisting of OCT3/4, SOX2, KLF4, LIN28, and c-Myc.

57. The method of any one of claims 23 to 56, wherein in step (d) the transduced γδ T cells are cultured in the presence of one or more feeder layers.

58. The method of claim 57, wherein in step (d) the transduced γδ T cells are cultured in the presence of a mono layer of feeder layer.

59. The method of claim 57 or 58, wherein the feeder layer comprises mouse embryonic fibroblasts (MEFs).

60. The method of any one of claims 23 to 59, further comprising isolating and/or purifying the produced iPSCs.

61. The method of any one of claims 23 to 60, further comprising differentiating the iPSCs ex vivo to cells of a desired cell type, thereby producing differentiated IPSCs.

62. The method of any one of claims 23 to 61, wherein the produced iPSCs are negative for a Sendai virus (SeV) vector.

63. The method of any one of claims 23 to 62, wherein the produced iPSCs are derived from γδ T cells.

64. The method of any one of claims 23 to 62, wherein the produced iPSCs have rearrangement genes of TRG and TRD gene loci; and wherein optionally the produced iPSCs have Vγ9 and Vδ2 gene arrangements.

65. The method of any one of claims 23 to 62, wherein the produced iPSCs are not derived from αβ T cells.

66. The method of any one of claims 23 to 62, wherein the produced iPSCs do not produce or express TCRA and or TCRB or fragments thereof, such that there is no surface expression of TCRA and TCRB, detectable or otherwise.

67. The method of any one of claims 23 to 62, wherein the produced iPSCs are genomically stable with no loss of a chromosome.

68. The method of claim 67, wherein the genomic stability of the produced iPSCs is determined by Karyotyping analysis.

69. The method of any one of claims 23 to 68, wherein the produced iPSCs can grow in feeder free medium after adoption.

70. An induced pluripotent stem cell (iPSC) produced according to the method of any one of claims 23 to 69.

71. The population of iPSCs of claim 1, wherein the iPSCs are produced according to the method of any one of claims 23 to 69.

72. A composition comprising the iPSC of claim 70.

73. A differentiated IPSC produced according to the method of claim 61.

74. A method of producing induced pluripotent stem cells (iPSCs) comprising:
(a) a step for performing a function of enriching and/or activating γδ T cells in an isolated population of cells;
(b) a step for performing a function of reprogramming the γδ T cells to a pluripotent state, thereby producing iPSCs; and
(c) a step for contacting the iPSCs with an RNA-guided endonuclease or a nucleic acid encoding the RNA-guided endonuclease and a guide RNA (gRNA);
wherein the gRNA binds to a target motif of a beta-2-microglobulin (B2M) polynucleotide sequence in the iPSCs; and
wherein the step for contacting results in cleavage of the B2M polynucleotide sequence.

75. An induced pluripotent stem cell (iPSC) produced according to the method of claim 74.

76. An isolated population of induced pluripotent stem cells (iPSCs) comprising pluripotent cells, wherein the pluripotent cells comprise a means for expressing one or more reprogramming factors, and/or wherein the pluripotent cells comprise a means for encoding rearrangement of TRG and TRD genes, and wherein the pluripotent cells comprise a means for cleaving a B2M gene.

77. The population of iPSCs of any one of claims 1-22, wherein the disruption further comprises a knock-in of a polynucleotide encoding a transgene, wherein the polynucleotide is inserted within the B2M gene.

78. The population of iPSCs of claim 77, wherein the transgene encodes a chimeric antigen receptor, a TCR, a therapeutic payload or a therapeutic protein.

79. The population of iPSCs of claim 77, wherein the polynucleotide is inserted within SEQ ID NO: 1.

80. The method of any one of claims 23-69, wherein the method further comprises introducing a vector comprising a gene encoding a transgene, wherein the gene is integrated into a target gene.

81. The method of claim 80, wherein the target gene comprises SEQ ID NO: 1.


SEQUENCE LISTING (24 sequences)

SEQ ID NO: 1 - DNA, 14401 bp, Homo sapiens
  source 1..14401: mol_type = genomic DNA
tttaaactgctgacaacttaacactgattactcatagaggcactgccatggtggtcttggataagatctggaagaattctctggattaccaggcagagactcttattgtcttcccttattttcccccaggcaaagtctgtctttctgtgatcagctgcctggagttgggggagaagtgacacaaacacccctgtgaccaccactgggactgtgcagggtcagacgtgaagccagcatagtactgggtcttgcccaaggcctatggtaaccaccgcctggctactacctatgttcactcaaggccctatggctctaaaatcagcaggtggcaaagccagccaagcttatgtccctctctttagggtggcaagtttcccccagcctggggtgaatccagagatgccttccaggaactggggcctagtcagaaaccttaggaatctacctggtactctattgcactgaggctgaactggcacccaagccacaagacaaagtccttcccacttttccctcccctttccacaagctgaggagtctcttgccatggccacttccaccccaggcctgtggtgggtactgtctggctactgctgatgtatactcaaggcccaagggctctgcagtaagcttgtggtgaacgctgcccagcctgggactcacccttcagggaagcaggctcccctctggcccagagcaggtccaggaatgctatctaagagccaaagcctggaatcagggaccttaagaggccacttggtattctaaccccacagtggccaagtcccctttactctttccacttcttttctcaagcagaaggaatttctccccatagccaccacagctgtaatgtgctgggtcacaccttaagcctgcacgtctctgagtctcacccaaagcccatgatgagtactacctggctacttctgattattcagggccaaagggctctttagtcagctggtgaggaatcctgccaggactggtccttcccttcaaggcagcaggttcccttctggcccagggtgtggctagaaatgtcagcttggagctatggcctggaatgtgggcctcaggactttgcctggtgccctatcctactgcagctgagctggtatccaatgcaagacaaactcatctttgttcttccctctcctcttctcaagcagaggaaagaagtctctctctgagctttgagctgccctgcctggggttgggagaggggtggtgcaagcaatcccttggccatcccagctagtgtctcactaggttgtgtgccccccacattcactggctctgagcccagcacagcaccaggacttacccaggaattgccatccttgtggcctagactgcctttcaagtttatgtagaacctcagagcacttcagcccatggtggggaggcttactggaactcaggttcccactgctgggatgggtgatttgcctctagctagggctggtctaaatgatccctccatgggacctgggtgagttcttcccagtgttgctttgtgctatgactgggcagcactgagttccaatgcaaagtctgaaaatcactgcactcttcctctcccagaagcacacattctctctccatgccaggtggccgctgccggcgaatgggggagagatagcatttagcaattcaagactgtcttaccctcttcagttactctttcagtgatacgaagttaaaaccaggtactgtgatcactcacctgatttttggttcttatgaaggtgcttttttttgtgtggatcattattcaatttggtgttcctgcagggagcaggattagtggaggcgtctattcagccatcttgctccacctcccctagctatgtccttttaaatatttataatcgacatacaatatattacacttaaagtatacaataagttctaaaatataactgaaatcatcatcgaactctagataattaacatatccatcaaccctaaagtttcctcatgtttagatgatctcctttccctctagaaaccactcatctactttctgtcatatagattatagtttgccttttctggagttttatataaacagaatcatatagtatatgttcttttctgtctgacttcttttatttaccatgattatttcatccatgttacatgtatcaacagtttattccttgtttgttgatgatagtatttcattgtatggatatatactccaatgggtttattcatttacctgttgatgtatgttggatcgtttctagttttcagctatcacaaataaaactgctataaacattaaacattaatgtccaagtctttgtatagacatatgctttcattttctcttgggcaaataccttgggtggagtggctggatattatggccagtgtatgtttaactttataaagagctgtcgaagtgttttcctttttcttttttctttttgagatgaagtcttgctctgttgcccaggctggagtgcagtggtgcaatctcggctcattgcaacctctgcctcccaggttcaagcaattctcctgcctcagcctcctgagtagttggggttacagatgcctgccaccatgcctagctaatttttgtatttttagtagagacagggtttcaccatgttggccaggctggtctcaaactcctgatctcaagtgatctgcccgccttgacctcccaaagtgctcagattacaggtgtgagccaccacatccagccccaaattgtttataccattttatattcctaccaggattgtatgagagttccagttcttctaaattcttgccaacatttactactttgaataaatgttcattgtaaaaaaattttaagagggttatataggggaaaaaaaggccgctattaatctgttaattgcactactgggagataaagctattaaactgtcatatttgtggctgggcgtggtaacacatgcttgtgatcctagcactttaggaggctggggaaggaggatcacttgagaccaggagttcgagaccagcctgggcaacacagcaagactctgtctccacaaaaatattaaaaattaaaaaaaaaagaaattgtcatgtttgtagacttcccaaatttgccatcctaggcaaacagtgctcttgtttttaaacaaatagttgtatacataattttccccctcactattcaaaacatgtcataaatggtaagtccaagaaaaatacaggtattcccccccaaagaaaactgtaaaatcgacttttttctatctgtactgttttttattggtttttaaattggttttccaagtgagtaaatcagaatctatctgtaatggattttaaatttagtgtttctctgtgatgtagtaaacaagaaactagaggcaaaaatagccctgtcccttgctaaacttctaaggcacttttctagtacaactcaacactaacatttcaggcctttagtgccttatatgagtttttaaaagggggaaaagggagggagcaagagtgtcttaactcatacatttaggcataacaattattctcatattttagttattgagagggctggtagaaaaactaggtaaataatattaataattatagcgcttattaaacactacagaacacttactatgtaccaggcattgtgggaggctctctcttgtgcattatctcatttcattaggtccatggagagtattgcattttcttagtttaggcatggcctccacaataaagattatcaaaagcctaaaaatatgtaaaagaaacctagaagttatttgttgtgctccttggggaagctaggcaaatcctttcaactgaaaaccatggtgacttccaagatctctgcccctccccatcgccatggtccacttcctcttctcactgttcctcttagaaaagatctgtggactccaccaccacgaaatggcggcaccttatttatggtcactttagagggtaggttttcttaatgggtctgcctgtcatgtttaacgtccttggctgggtccaaggcagatgcagtccaaactctcactaaaattgccgagccctttgtcttccagtgtctaaaatattaatgtcaatggaatcaggccagagtttgaattctagtctcttagcctttgtttcccctgtccataaaatgaatgggggtaattctttcctcctacagtttatttatatattcactaattcattcattcatccatccattcgttcattcggtttactgagtacctactatgtgccagcccctgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctgatggccagctttgtttatttgaccaagaatctgagttagctagttctaggtactgacaggataaataaaacacaacactgctcccgatcttctcagtttagcagagggacagatatgcactcaaataattaaaatatatcctgataagaatatagcataggtacgcgcgaagaacttggcaatcgaaattttgttgttcaggctgggcgaggtgtctcatgtctgtaatcccagcactttgggaggccatggtaggatgatcgcttgagcccaggagtccgagaccagcctgggcaacataacaagaccctgtctcaattcaaagaattgaattaaaaaaaacaaaaaataatttttttaaaaaagaaatgttgttgttcaaggaacaacaacaaaaatctagggaggtgttagagaagccatttgcctgagctgagagtaagttgctagtggttctcttgattggtaggtggggcctgggtttccaggcatggtagccaggaaggacagccacatggcaggtttgggtaattccaaacagtggaggaagggtgtctgggggaagacttgtaggaactcagctgaaaaaattgggggatgatactctgaaagaaaaacaaagttttaaaatttctactcttacacttaacacataatgcttctgtgaccggatatttaggggttttccccccacactctgttaggagaaaaattttagacagattaaatttaacagagtttaactgagcaaaaatgattctcgaaccaggcagctgccggagccagaataggttcaaaatgactctgggggtgccacatggttggatgacatttagggacagaaaaaggaaagtgatgtgcagaaaatggaagtcaggggcagaagcagccagattggttgcagttcagcatttgcctcatttaaacagggtttgaagagttggccacctgtgattggctgagactctgtggtataagagtaagttacagtctgtttacacatccagttaggttacagttcactatgcagagagaaatctttagcctgaacttacacagggaggcagttttatttatttatttaattttttttttttgagacaaggtctcactctgtcacccgggctggagttcagtggtatgatcatggcttattgcagcctcgacttcctggcctcaagcaatccttccgcctcaggctctagagtagctgggactacaggcacatgtcagcatgcctggctaattttgttttttaatttttagtagagatgaactcctggccttgcacaattctttcgcctcagcctccgaaaatgctgggattacaggtgtgagccactgtgcccagctaaggcaactttaggctaaacctttttttgagacagagtttctctcttgttgtctaggctggagtgcagttgcaccatcttggctcactgcaacctccacctccagggttcaagtgattctcgttcctcagcctcccgagtaactgggattacaggcatgcgccaccacgcctggctaattttgtgtttttagtagagatggtgtttcaccatgttgtccaggctggtctcaaactcctggcctcaagtgatcctctggcctcagcttcccagagtactgagattacaggcatgagccactgtgccctgcctaggctaaacttaatttaacaacaccaaacaatctccagcagacaccaactgggtatcccataattcaattcgattttgattggatctacctggagatggtgtcagatcccgctggttgagggttcagtcccacaagactgccctccacttcagatgccaatcacacattgtaggttgttacctctacttctgactgaccagctggaaaccagaactcccatgactgcctccttgactttggttaatttgctaggacagttcatatttaccaatctattataaaagattaaaggctacagacgaataactagatgaaaagatgaatagggctatatgt

SEQ ID NO: 2 - DNA, 21 bp, Homo sapiens
  source 1..21: mol_type = genomic DNA
atccatccgacattgaagttg

SEQ ID NO: 3 - DNA, 20 bp, Homo sapiens
  source 1..20: mol_type = genomic DNA
tcacagcccaagatagttaa

SEQ ID NO: 4 - DNA, 21 bp, Homo sapiens
  source 1..21: mol_type = genomic DNA
ctcacgtcatccagcagagaa

SEQ ID NO: 5 - DNA, 21 bp, Homo sapiens
  source 1..21: mol_type = genomic DNA
gagtacgctggatagcctcca

SEQ ID NO: 6 - DNA, 24 bp, Homo sapiens
  source 1..24: mol_type = genomic DNA
agtgggggtgaattcagtgtagta

SEQ ID NO: 7 - RNA, 41 nt, synthetic construct
  source 1..41: mol_type = other RNA
taatttctactcttgtagatatccatccgacattgaagttg

SEQ ID NO: 8 - RNA, 40 nt, synthetic construct
  source 1..40: mol_type = other RNA
taatttctactcttgtagattcacagcccaagatagttaa

SEQ ID NO: 9 - RNA, 41 nt, synthetic construct
  source 1..41: mol_type = other RNA
taatttctactcttgtagatctcacgtcatccagcagagaa

SEQ ID NO: 10 - RNA, 41 nt, synthetic construct
  source 1..41: mol_type = other RNA
taatttctactcttgtagatgagtacgctggatagcctcca

SEQ ID NO: 11 - RNA, 44 nt, synthetic construct
  source 1..44: mol_type = other RNA
taatttctactcttgtagatagtgggggtgaattcagtgtagta

SEQ ID NO: 12 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 13 - protein, 101 aa, Homo sapiens
MSRSVALAVLALLSLSGLEAIQRTPKIQVYSRHPADIEVDLLKNGERIEKVEHSDLSFSKDWSFYLLYYTEFTPTEKDEYACRVNHVTLSQPKIVKWDRDM

SEQ ID NO: 14 - protein, 71 aa, Homo sapiens
GFHPSDIEVDLLKNGERIEKVEHSDLSFSKDWSFYLLYYTEFTPTEKDEYACRVNHVTLSQPKIVKWDRDM

SEQ ID NO: 15 - protein, 57 aa, Homo sapiens
MSRSVALAVLALLSLSGLERELKKWSIQTCLSARTGLSISCTTLNSPPLKKMSMPAV

SEQ ID NO: 16 - protein, 51 aa, Homo sapiens
MSRSVALAVLALLSLSGLEAIQHSGLLTSSSREWKVKFPELLCVWVSSIRH

SEQ ID NO: 17 - DNA, 21 bp, Homo sapiens
  source 1..21: mol_type = genomic DNA
agtgggggtgaattcagtgta

SEQ ID NO: 18 - RNA, 41 nt, synthetic construct
  source 1..41: mol_type = other RNA
taatttctactcttgtagatagtgggggtgaattcagtgta

SEQ ID NO: 19 - DNA, 21 bp, Homo sapiens
  source 1..21: mol_type = genomic DNA
caacttcaatgtcggatggat

SEQ ID NO: 20 - DNA, 20 bp, Homo sapiens
  source 1..20: mol_type = genomic DNA
ttaactatcttgggctgtga

SEQ ID NO: 21 - DNA, 21 bp, Homo sapiens
  source 1..21: mol_type = genomic DNA
ttctctgctggatgacgtgag

SEQ ID NO: 22 - DNA, 21 bp, Homo sapiens
  source 1..21: mol_type = genomic DNA
tggaggctatccagcgtactc

SEQ ID NO: 23 - DNA, 24 bp, Homo sapiens
  source 1..24: mol_type = genomic DNA
tactacactgaattcacccccact

SEQ ID NO: 24 - DNA, 21 bp, Homo sapiens
  source 1..21: mol_type = genomic DNA
tacactgaattcacccccact